(19) United States
(12) Patent Application Publication
(10) Pub. No.: US 2009/0082328 A1
(43) Pub. Date: Mar. 26, 2009

(54) Substituted phenylamino-benzene derivatives useful for treating hyper-proliferative disorders and diseases associated with mitogen extracellular kinase activity

(75) Inventors: Yingfu Li, Hamden, CT (US); Joachim Rudolph, Burlingame, CA (US); Jacques P. Dumas, Waltham, MA (US); Daniel Auclair, Middletown, CT (US); Mario Lobell, Wuppertal (DE); Marion Hitchcock, Berlin (DE); Ingo Hartung, Berlin (DE); Marcus Koppitz, Berlin (DE); Dominic Brittain, Berlin (DE); Florian Puehler, Berlin (DE); Kirstin Petersen, Berlin (DE); Judith Guenther, Berlin (DE)

Correspondence Address: Edwards Angell Palmer & Dodge LLP, P.O. Box 55874, Boston, MA, 02205, US

(73) Assignees: Bayer Schering Pharma Aktiengesellschaft; Bayer HealthCare AG

(21) Appl. No.: 12/151,855

(22) Filed: May 9, 2008

Related U.S. Application Data
(60) Provisional application No. 60/928,789, filed on May 11, 2007.

(30) Foreign Application Priority Data
Apr. 22, 2008 (EP) EP08075314.8

(51) Int. Cl. A61K 31/445; C07C 205/45; C07D 211/32; C07C 255/58; C07D 317/30; C07C 237/28; A61K 31/397; A61K 31/40; A61K 31/27; A61P 35/00; A61K 31/44; A61K 31/166; C07D 295/14; C07D 279/12; C07D 265/30; A61K 31/54; A61K 31/5375

(52) U.S. Cl. 514/210.17; 564/433; 546/232; 558/418; 549/452; 564/167; 558/413; 558/417; 544/398; 544/58.2; 544/163; 514/227.5; 514/238.8; 514/330; 514/619; 514/424; 514/357; 514/486; 514/600

(57) ABSTRACT

This invention relates to novel substituted phenylamino-benzene compounds, pharmaceutical compositions containing such compounds and the use of those compounds or compositions for treating hyper-proliferative and/or angiogenesis disorders, as a sole agent or in combination with other active ingredients.

### RELATED APPLICATIONS

This is a utility patent application which claims priority to EP patent application No. EP08075314.8, filed on Apr. 22, 2008, and claims priority to U.S. Provisional patent application No. 60/928,789, filed on May 11, 2007. The entire teachings of the above applications are incorporated herein by reference.

### FIELD OF THE INVENTION

This invention relates to novel substituted phenylamino-benzene compounds, pharmaceutical compositions containing such compounds and the use of those compounds or compositions for treating hyper-proliferative and/or angiogenesis disorders, as a sole agent or in combination with other active ingredients.

### BACKGROUND OF THE INVENTION

Cancer is a disease resulting from an abnormal growth of tissue. Certain cancers have the potential to invade into local tissues and also metastasize to distant organs. This disease can develop in a wide variety of different organs, tissues, and cell types. Therefore, the term “cancer” refers to a collection of over a thousand different diseases.

Over 4.4 million people worldwide were diagnosed with breast, colon, ovarian, lung, or prostate cancer in 2002 and over 2.5 million people died of these devastating diseases (Globocan 2002 Report). In the United States alone, over 1.25 million new cases and over 500,000 deaths from cancer were predicted in 2005. The majority of these new cases were expected to be cancers of the colon (−100,000), lung these new cases were expected to be cancers of the colon (−100,000), lung (−170,000), breast (−210,000) and prostate (−230,000). Both the incidence and prevalence of cancer is predicted to increase by approximately 15% over the next ten years, reflecting an average growth rate of 1.4% [1].

Accumulating evidence suggests that cancer can be envisioned as a “signaling disease”, in which alterations in the cellular genome affecting the expression and/or function of oncogenes and tumor suppressor genes would ultimately affect the transmission of signals that normally regulate cell growth, differentiation, and programmed cell death (apoptosis). Unraveling the signaling pathways that are dysregulated in human cancers has resulted in the design of an increasing number of mechanism-based therapeutic agents [2]. Signal transduction inhibition as a therapeutic strategy for human malignancies has recently met with remarkable success, as exemplified by the development of Gleevec for the treatment of chronic myelogenous leukemia (CML) and gastrointestinal stromal tumors (GIST), heralding a new era of “molecularly-targeted” therapies [3-5].

The mitogen-activated protein kinase (MAPK) module is a key integration point along the signal transduction cascade that links diverse extracellular stimuli to proliferation, differentiation and survival. Scientific studies over the last twenty years have led to a quite detailed molecular dissection of this pathway, which has now grown to include five different MAPK subfamilies [extracellular signal-regulated kinases ERK-1/2, c-Jun-N-terminal kinases (JNKs), p38 kinases, ERK-3/4, and ERK-5], with distinct molecular and functional features [6-8]. While certain subfamilies, such as the p38 family, are becoming therapeutic targets in inflammatory and degenerative diseases, the MAPK cascade that proceeds from Ras to ERK-1/2 (the main mitogenic pathway initiated by peptide growth factors) is starting to emerge as a prime target for the molecular therapy of different types of human cancers [9-11], The MAPK pathway is aberrantly activated in many human tumors as a result of genetic and epigenetic changes, resulting in increased proliferation and resistance to apoptotic stimuli. In particular, mutated oncogenic forms of Ras are found in 50% of colon and >90% of pancreatic cancers [12]. Recently, BRAF mutations have been found in >60% of malignant melanoma [13]. These mutations result in a constitutively activated MAPK pathway. In addition, overexpression of or mutational activation of certain receptor tyrosine kinases can also lead to increased activation of the Raf-MEK-ERK pathway.

The modular nature of the Raf/MEK/ERK cascade becomes less pleiotropic at the crossover point that is regulated by MEK [14]. No substrates for MEK have been identified other than ERK-1/2. Phosphorylated ERK is the product of MEK activity and thus its detection in cancer cells and in tumor tissues provides a direct measure of MEK inhibition. The selectivity of MEK for ERK1/2 coupled with the availability of antibodies specific for the dually phosphorylated and activated form of ERK, makes MEK an attractive target for anticancer drug development. In addition, it was recently shown that MEK activation regulates matrix mineralization (Blood 2007, 40, 68), thereby modulation of MEK activity may also be applicable for the treatment of diseases caused by or accompanied with dysregulation of tissue mineralization, more specifically for the treatment of diseases caused by or accompanied with dysregulation of bone mineralization.

First-generation MEK inhibitors, PD98059 [15] and U0126 [16], do not appear to compete with ATP and thus are likely to have distinct binding sites on MEK; these compounds have been extensively used in model systems in vitro and in vivo to attribute biological activities to ERK1/2. A second-generation MEK1/2 inhibitor, PD184352 (now called CI-1040), has an IC₅₀ in the low nanomolar range, enhanced bioavailability, and also appears to work via an allosteric, non ATP-competitive mechanism [17]. Oral treatment with CI-1040 has been shown to inhibit colon cancer growth in vivo in mouse models [18] and this compound was evaluated in phase I/II clinical trials in humans where it eventually failed because of insufficient efficacy [19]. Further allosteric MEK inhibitors have recently entered the clinic but were found to have limitations such as poor exposure profiles, limited efficacy and/or toxicity issues. Small molecules MEK inhibitors have been disclosed, including in US Patent Publications Nos. 2003/0232869, 2004/0116710, 2003/0216420 and in U.S. patent applications Ser. Nos. 10/654,580 and 10/929,295 each of which is hereby incorporated by reference. A number of additional patent applications have appeared in the last few years including U.S. Pat. No. 5,525,6625; WO 98/43960; WO 99/01421; WO 99/01426; WO 00/41505; WO 00/41994; WO 00/42002; WO 00/42003; WO 00/42022; WO 00/42029; WO 00/68201; WO 01/68619; WO 02/06213; WO 03/077914; WO 03/077855; WO 04/083167; WO 05/0281126; WO 05/051301; WO 05/121142; WO 06/114466; WO 98/37881; WO 00/35435; WO 00/35436; WO 00/40235; WO 00/40237; WO 01/05390; WO 01/05391; WO 01/05392; WO 01/05393; WO 03/062189; WO 03/062191; WO 04/056789; WO 05/000818; WO 05/007616; WO 05/009975; WO 05/051300; WO05/051302; WO 05/028426; WO 06/056427; WO 03/035626; and WO 06/029862.

Despite advancements in the art, there remains a need for cancer treatments and anti-cancer compounds. More specifically, there remains a need for structurally novel MEK inhibitors with a balanced potency-properties profile. It would be especially desirable to identify novel MEK inhibitors which incorporate structural motifs which have not been previously exemplified as being compatible with potent MEK inhibition. It would be especially favorable if these structural motifs would further allow for improvement of MEK potency and/or modulation of compound properties (including physico-chemical, pharmacodynamical and pharmacokinetical properties).

It is now found that compounds of the present invention are potent and selective MEK inhibitors. The compounds of the present invention are derived from a 1-substituted-2-phenylamino-phenyl scaffold with a further specifically substituted side chain in the 6-position of the phenyl scaffold. This finding is surprising as inspection of published phenyl-scaffold-derived MEK inhibitors and previous structure-activity relationship analysis (see for example Haile Tecle/Pfizer Global Research: “MEK inhibitors”, presented at Drew University, 15^{th} Jun. 2006) suggested that in phenyl-scaffold-based MEK inhibitors larger 6-substituents are detrimental for achieving high MEK inhibitory potency. Compounds of the present invention are potent MEK inhibitors and inhibit activation of the MEK-ERK pathway. Compounds and compositions described herein, including salts, metabolites, solvates, solvates of salts, hydrates, prodrugs such as esters, polymorphs, and stereoisomeric forms thereof, exhibit anti-proliferative activity and are thus useful to prevent or treat the disorders associated with hyper-proliferation.

### DESCRIPTION OF THE INVENTION

The present invention thus relates to compounds of general formula (I):

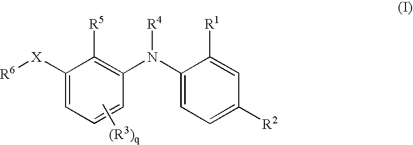

in which
R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom
each occurrence of R³ is independently a halogen atom, a C₁-C₄-alkyl or —CN group
q is an integer of 0, 1, 2, or 3;
R⁴ is a hydrogen atom or a C₁-C₆-alkyl group;
R⁵ is a —C(═O)R⁷, —C(═O)OR⁷, —C(═O)N(R⁷)(R⁸), —NHC(═O)R⁷, —S(═O)₂R⁷, —NHS(═O)₂R⁷, —S(═O)₂NR⁷R⁸, —NO₂, —CN, or a

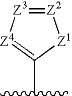

group,
in which:
each of Z¹, Z², Z³ and Z⁴ is independently —CH—, —C(C₁-C₆-alkyl)-, —C(═O)—, —S—, —O—, —N— or —NH, such that at least one of Z¹, Z², Z³ and Z⁴ is —N— or —NH—;
X is —O—, —NH—, —N(C₁-C₆-alkyl)-, —S—, —S(═O)₂—, —C(═O)—, —C(═O)O—, —C(═O)NH—, or NHC(═O)—;
R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R¹⁰, —(CH₂)ₙ—(CHN((R¹²)(R¹³)))—(CH₂)ₘ—R¹⁰, —(CR¹⁵₂)ₙ—(CR¹⁵N((R¹²)(R¹³)))—(CR¹⁵₂)ₘ—R⁹, —(CH₂)ₙ—Y, (CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), aryl, heteroaryl, C₂-C₁₀-alkenyl, C₅-C₁₀-cycloalkenyl, cycloalkyl or heterocycloalkyl group, in which aryl, heteroaryl, cycloalkyl, or heterocycloalkyl is optionally substituted with one or more —(CH₂)ₒR¹⁴ groups;
R⁷ and R⁸ are independently a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹, R¹² and R¹³ are independently a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is, independently, a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NHS(═O)₂H, —NR^{a}S(═O)₂R^{b}, —S(═O)₂R^{b} or —C(═O)R^{b} group;
each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2;
each occurrence of R^{a} is, independently, a hydrogen atom or a C₁-C₆-alkyl group;
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

In accordance with an embodiment, the present invention relates to compounds of formula (I), supra, in which:

R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group;
q is an integer of 0, 1, 2, or 3;
R⁴ is a hydrogen atom or a C₁-C₆-alkyl group;

### R⁵ is a —C(═O)R⁷

R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R⁹, —(CH₂)ₙ—(CHN((R¹²)(R¹³)))—(CH₂)ₘ—R¹⁰, —(CR¹⁵₂)ₙ—(CR¹⁵N((R¹²)(R¹³)))—(CR¹⁵₂)ₘ—R¹⁰, —(CH₂)ₙ—Y, —(CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), aryl, heteroaryl, C₂-C₁₀-alkenyl, C₅-C₁₀-cycloalkenyl, cycloalkyl or heterocycloalkyl group, in which aryl, heteroaryl, cycloalkyl, or heterocycloalkyl is optionally substituted with one or more —(CH₂)ₒR¹⁴ groups;
R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group, in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups
each occurrence of R¹⁴ is a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NR^{a}S(═O)₂R^{b}, —S(═O)₂R^{b} or —C(═O)R^{b} group;
each occurrence of R¹⁵ is, independently, a hydrogen atom or a C₁-C₆-alkyl group
each occurrence of n is, independently, an integer of 0, 1, 2, 3, or 4;
each occurrence of m is, independently, an integer of 0, 1, or 2; and
each occurrence of o is, independently, an integer of 0, 1, or 2
each occurrence of R^{a} is, independently, a hydrogen atom or a C₁-C₆-alkyl group
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in is which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR)₂ group
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2} C₁-C₆-alkyl, cycloalkyl, C₁-C₆-alkoxy, aryl or heteroaryl group
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that
X-R⁶ is not (O or NH)—(CH₂)ᵣ—R^{r}
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

In accordance with a preferred embodiment, the present invention relates to compounds of formula (I), supra, in which

R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group;
q is an integer of 0, 1, 2, or 3;
R⁴ is a hydrogen atom or a C₁-C₆-alkyl group

### R⁵ is a —C(═O)R⁷

R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R⁹, —(CH₂)ₙ—(CHN((R¹²) (R¹³)))—(CH₂)ₘ—R¹⁰, —(CR¹⁵₂)ₙ—(CR¹⁵N((R¹²)(R¹³)))—(CR¹⁵₂)ₘ—R¹⁰, —(CH₂)ₙ—Y, —(CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), C₂-C₁₀-alkenyl, C₅-C₁₀-cycloalkenyl, cycloalkyl or heterocycloalkyl group, in which cycloalkyl or heterocycloalkyl is optionally substituted with one or more —(CH₂)ₒR¹⁴ groups;
R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))-(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group, in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₙR¹⁴ groups;
each occurrence of R¹⁴ is a halogen atom, C₁-C₆ alkoxy, C₁-C₆ alkylamino or (C₁-C₆-alkyl)₂-amino;
each occurrence of R¹⁵ is, independently, a hydrogen atom or a C₁-C₆-alkyl group
each occurrence of n is, independently, an integer of 0, 1, 2, 3, or 4;
each occurrence of m is, independently, an integer of 0, 1, or 2; and
each occurrence of o is, independently, an integer of 0, 1, or 2
each occurrence of R^{a} is, independently, a hydrogen atom or a C₁-C₆-alkyl group
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR¹)₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that:
X-R⁶ is not (O or NH)—(CH₂)ᵣ—R^{r}
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

In accordance with a further preferred embodiment, the present invention relates to compounds of formula (I), supra, in which

R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group;
q is an integer of 0, 1, 2, or 3;
R⁴ is a hydrogen atom or a C₁-C₆-alkyl group

### R⁵ is a —C(═O)R⁷

R⁶ is —(CH₂)ₙ—Y;
Y is aryl, heteroaryl, in which aryl, heteroaryl is optionally substituted with one or more —(CH₂)ₙR¹⁴ groups;
R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group, in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NR^{a}S(═O)₂R^{b}, —S(═O)₂R^{b} or —C(═O)R^{b} group
a halogen atom, C₁-C₆ alkoxy, C₁-C₆ alkylamino or (C₁-C₆-alkyl)₂-amino;
each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2
each occurrence of R^{a} is, independently, a hydrogen atom or a C₁-C₆-alkyl group
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group; in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2} are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that:
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

One embodiment of this invention encompasses a compound having the formula (I):

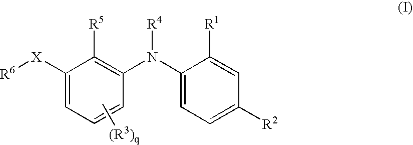

or a physiologically acceptable salt, solvate, hydrate or stereoisomer thereof, wherein:
q is an integer from 0-3
R¹ and R² may be the same or different and are independently hydrogen, halogen, (C₁-C₆) alkyl, (C₂-C₆) alkenyl, (C₂-C₆) alkynyl, or —CN, wherein at least one of R¹ and R² is halogen;
each occurrence of R³ is independently halogen, (C₁-C₄)alkyl or —CN;
R⁴ is hydrogen or (C₁-C₆) alkyl;
R⁵ is —COR⁷, —COOR⁷, —CON(R⁷) (R⁸), —NH—(CO)—R⁷, —SO₂(R⁷), —NHSO₂(R⁷), —SO₂N (R⁷)(R⁸), —NO₂, —CN, or

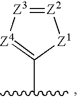

wherein:
   each of Z¹, Z², Z³ and Z⁴ is independently —CH, —C[(C₁-C₆) alkyl]-, —Co—, —S—, —O—, —N— or —NH such that at least one of Z¹, Z², Z³ and Z⁴ is —N— or —NH;
X is —O—, —NH—, —N(C₁-C₆)alkyl-, —S—, —SO₂—, —CO—, —COO—, —CONH—,
or —NHCO—;
R⁶ is —(CH₂)ₙ—(CH(OR¹¹))—(CH₂)ₘ—R⁹,
   —(CH₂)ₙ—(CHN((R¹²)(R¹³)))—(CH₂)ₘ—R¹⁰,
   —(CH₂)ₙ—Y,
   —(CH₂)ₙ—(CHOH)—(CHOH)—(CH₂OH), or
   —(CH₂)ₙ—(CHOH)—(COOH);
Y is hydroxy, —SO₂NH₂, —SO₂NH((C₁-C₃)alkyl), —N(R¹²)(R¹³), aryl, heteroaryl, cycloalkyl or heterocycloalkyl, wherein aryl, heteroaryl, cycloalkyl, or heterocycloalkyl is optionally substituted with one or more R¹⁴ groups;
R⁷ and R⁸ are independently hydrogen, —N(R¹²)(R¹³), hydroxy, —(C₁-C₆)alkoxy, —(C₁-C₆)alkyl, —CF₃, —O—(CH₂)ₙ—(CH(OR¹¹))—(CH₂)ₘ—R⁹, —O—(CH₂)ₙ-cycloalkyl, -aryl, heteroaryl, cycloalkyl or heterocycloalkyl, wherein aryl, heteroaryl, cycloalkyl, or heterocycloalkyl is optionally substituted with one or more halogen, —(C₁-C₆)alkyl or —(C₁-C₆)alkoxy groups;
R⁹ and R¹⁰ are independently hydroxy, —(C₁-C₆)alkoxy or —N(R¹²)(R¹³)
R¹¹, R¹² and R¹³ are independently
   hydrogen, —(C₁-C₆)alkyl, aryl, heteroaryl, cycloalkyl or heterocycloalkyl, wherein aryl, heteroaryl, cycloalkyl, or heterocycloalkyl is optionally substituted with one or more R¹⁴ groups,
   or
   R¹² and R¹³ can be taken together with the N atom connecting them to form a 5-7 membered heterocyclic ring optionally comprising one or more additional heteroatoms and which is optionally substituted with one or more R¹⁴ groups;
each occurrence of R¹⁴ is independently, hydroxy, —(C₁-C₆)alkoxy, amino, alkylamino, dialkylamino, halo, cyano, —NHSO₂H, —SO₂-amino, —NHSO₂-alkyl, —SO₂-alkylamino, —SO₂-dialkylamino;
each occurrence of n is independently an integer from 0-4; and each occurrence of m is independently an integer from 0-2.

In a preferred embodiment, the invention encompasses the compound of Formula (I),

wherein R² is halogen and R¹ is halogen, (C₁-C₆) alkyl, (C₂-C₆) alkenyl, (C₂-C₆) alkynyl or —CN. More preferrably R² is iodine or bromine.

In another preferred embodiment, the invention encompasses the compound of Formula (I), wherein R¹ and R² may be the same or different and are both halogen, more preferrably wherein R¹ is fluorine and R² is iodine or bromine.

In still another preferred embodiment, the invention encompasses the compound of Formula (I), wherein R³ is fluorine, chlorine or methyl.

In yet another preferred embodiment, the invention encompasses the compound of Formula (I), wherein R⁴ is hydrogen.

In another embodiment, the invention encompasses the compound of Formula (I), wherein R⁶ is —(CH₂)ₙ—(CHOH)—(CH₂)ₘ—R⁹.

In still another embodiment, the invention encompasses the compound of Formula (I), wherein R⁹ is hydroxy or amino.

In a distinct embodiment, the invention encompasses the compound of Formula (Ia), having the formula:

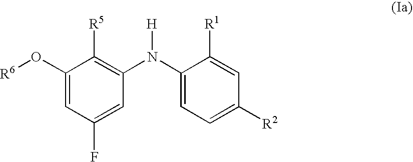

wherein
R¹ is hydrogen, halogen, (C₁-C₆) alkyl, (C₂-C₆) alkenyl, (C₂-C₆) alkynyl, or —CN,
R² is iodine or bromine;
R⁵ is —CONH₂, —NO₂, or —CN;
R⁶ is —(CH₂)ₙ—(CH(OR¹¹))—(CH₂)ₘ—R⁹,
   —(CH₂)ₙ (CHN((R¹²)(R¹³)))—(CH₂)ₘ—R¹⁰,
   —(CH₂)ₙ—Y,
   —(CH₂)ₙ—(CHOH)—(CHOH)—(CH₂OH), or
   —(CH₂)ₙ—(CHOH)—(COOH);
Y is hydroxy, —SO₂NH₂, —SO₂NH((C₁-C₃)alkyl), —N(R¹²)(R¹³), aryl, heteroaryl, cycloalkyl or heterocycloalkyl, wherein aryl, heteroaryl, cycloalkyl, or heterocycloalkyl is optionally substituted with one or more R¹⁴ groups
R⁹ and R¹⁰ are independently hydroxy, —(C₁-C₆)alkoxy or —N(R¹²)(R¹³);
R¹¹, R¹² and R¹³ are independently hydrogen, —(C₁-C₆)alkyl, aryl, heteroaryl, cycloalkyl or heterocycloalkyl, wherein aryl, heteroaryl, cycloalkyl, or heterocycloalkyl is optionally substituted with one or more R¹⁴ groups, or R¹² and R¹³ can be taken together with the N atom connecting them to form a 5-7 membered heterocyclic ring optionally comprising one or more additional heteroatoms and which is optionally substituted with one or more R¹⁴ groups;
each occurrence of R¹⁴ is independently, hydroxy, —(C₁-C₆)alkoxy, amino, alkylamino, dialkylamino, halo, cyano, —NHSO₂H, —SO₂-amino, —NHSO₂-alkyl, —SO₂-alkylamino, —SO₂-dialkylamino;
each occurrence of n is independently an integer from 0-4; and
   each occurrence of m is independently an integer from 0-2.

In a preferred embodiment, the invention encompasses the compound of Formula (Ia),

wherein R⁶ is —(CH₂)ₙ—(CHOH)—(CH₂)ₘ—R⁹.

In another preferred embodiment, the invention encompasses the compound of Formula (Ia), wherein R⁹ is hydroxy or amino.

In a separate embodiment, the invention encompasses a compound having the chemical name:
5-fluoro-3-[(2-fluoro-4-iodophenyl)amino]-2-nitrophenoxybutane-1,2-diol;
5-fluoro-N-(2-fluoro-4-iodophenyl)-2-nitro-3-(2-piperidin-4-ylethoxy)aniline
2-(3,4-dihydroxybutoxy)-4-fluoro-6-[(2-fluoro-4-iodophenyl)amino]benzonitrile
2-[2-(2,2-dimethyl-1,3-dioxolan-4-yl)ethoxy]-4-fluoro-6-[(2-fluoro-4-iodophenyl)amino]benzamide;
2-(3,4-dihydroxybutoxy)-4-fluoro-6-[(2-fluoro-4-iodophenyl)amino]benzamide
5-fluoro-3-[(2-fluoro-4-iodophenyl)amino]-2-nitrophenoxypropane-1,2-diol;
5-fluoro-3-[(2-fluoro-4-iodophenyl)amino]-2-nitrophenoxypentane-1,2-diol;
2-(2,3-dihydroxypropoxy)-4-fluoro-6-[(2-fluoro-4-iodophenyl)amino]benzonitrile;
2-[(4,5-dihydroxypentyl)oxy]-4-fluoro-6-[(2-fluoro-4-iodophenyl)amino]benzonitrile;
2-[(4S)-2,2-dimethyl-1,3-dioxolan-4-yl]propoxy-4-fluoro-6-[(2-fluoro-4-iodophenyl)amino]benzamide;
2-[(3R)-3,4-dihydroxybutyl]oxy-4-fluoro-6-[(2-fluoro-4-iodophenyl)amino]benzamide;
2-[(3S)-3,4-dihydroxybutyl]oxy-4-fluoro-6-[(2-fluoro-4-iodophenyl)amino]benzamide;
2-[(4S)-4,5-dihydroxypentyl]oxy-4-fluoro-6-[(2-fluoro-4-iodophenyl)amino]benzamide;
2-{[(3R)-3,4-dihydroxybutyl]oxy}-4-fluoro-6-[(4-iodophenyl)amino]benzamide
2-[(2-chloro-4-iodophenyl)amino]-6-{[(3R)-3,4-dihydroxybutyl]oxy}-4-fluorobenzamide;
2-{[(3R)-3,4-dihydroxybutyl]oxy}-4-fluoro-6-[(4-iodo-2-methylphenyl)amino]benzamide
2-[(2-cyano-4-iodophenyl)amino]-6-{[(3R)-3,4-dihydroxybutyl]oxy}-4-fluorobenzamide;
2-[(4-bromo-2-fluorophenyl)amino]-6-{[(3R)-3,4-dihydroxybutyl]oxy}-4-fluorobenzamide;
2-[(4-bromo-2-chlorophenyl)amino]-6-{[(3R)-3,4-dihydroxybutyl]oxy}-4-fluorobenzamide;
2-{[(3R)-3,4-dihydroxybutyl]oxy}-6-[(4-ethynyl-2-fluorophenyl)amino]-4-fluorobenzamide;
2-{[(3R)-3,4-dihydroxybutyl]oxy}-4-fluoro-6-[(2-fluoro-4-iodophenyl)amino]-N-methylbenzamide;
2-{[(3R)-3,4-dihydroxybutyl]oxy}-N-ethyl-4-fluoro-6-[(2-fluoro-4-iodophenyl)amino]benzamide;
2-{[(3R)-3,4-dihydroxybutyl]amino}-4-fluoro-6-[(2-fluoro-4-iodophenyl)amino]benzamide;
2-{[(3R)-3,4-dihydroxybutyl](methyl)amino}-4-fluoro-6-[(2-fluoro-4-iodophenyl)amino]benzamide;
4-fluoro-2-[(2-fluoro-4-iodophenyl)amino]-6-{[(2S,3S)-2,3,4-trihydroxybutyl]oxy}benzamide; or
4-fluoro-2-[(2-fluoro-4-iodophenyl)amino]-6-{[(2R,3R)-2,3,4-trihydroxybutyl]oxy}benzamide;

or a physiologically acceptable salt, solvate, hydrate or stereoisomer thereof.

### DEFINITIONS

The term “alkyl” refers to a straight or branched hydrocarbon chain radical consisting solely of carbon and hydrogen atoms, containing solely carbon and hydrogen atoms, containing no unsaturation, having from one to eight carbon atoms, and which is attached to the rest of the molecule by a single bond, such as illustratively, methyl, ethyl, n-propyl, 1-methylethyl (isopropyl), n-butyl, n-pentyl, and 1,1-dimethylethyl (t-butyl).

The term “alkenyl” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, 2- and butenyl.

The term “alkynyl” refers to a straight or branched chain hydrocarbonyl radicals having at least one carbon-carbon triple bond, and having in the range of about 2 up to 12 carbon atoms (with radicals having in the range of about 2 up to 10 carbon atoms presently being preferred) e.g., ethynyl.

The term “alkoxy” denotes an alkyl group as defined herein attached via oxygen linkage to the rest of the molecule. Representative examples of those groups are methoxy and ethoxy.

The term “alkoxyalkyl” denotes an alkoxy group as defined herein attached via oxygen linkage to an alkyl group which is then attached to the main structure at any carbon from alkyl group that results in the creation of a stable structure at the rest of the molecule. Representative examples of those groups are —CH₂OCH₃, and —CH₂OC₂H₅.

The term “cycloalkyl” denotes a non-aromatic mono or multicyclic ring system of about 3 to 12 carbon atoms such as cyclopropyl, cyclobutyl, cyclopentyl, cyclohexyl and examples of multicyclic cycloalkyl groups include perhydronaphthyl, adamantyl and norbornyl groups bridged to a cyclic group or sprirobicyclic groups e.g. spiro (4,4) non-2-yl. The term “cycloalkyl” is to be understood as preferably meaning a C₃-C₁₂ cycloalkyl group, more particularly a saturated cycloalkyl group of the indicated ring size, meaning e.g. a cyclopropyl, cyclobutyl, cyclopentyl, cyclohexyl, cycloheptyl, cyclooctyl, cyclononyl, or cyclodecyl group; and also as meaning an unsaturated cycloalkyl group containing one or more double bonds in the C-backbone, e.g. a C₃-C₁₀ cycloalkenyl group, such as, for example, a cyclopropenyl, cyclobutenyl, cyclopentenyl, cyclohexenyl, cycloheptenyl, cyclooctenyl, cyclononenyl, or cyclodecenyl group, wherein the linkage of said cyclolalkyl group to the rest of the molecule can be provided to the double or single bond; and also as meaning such a saturated or unsaturated cycloalkyl group being optionally substituted one or more times, independently of each other, with a C₁-C₆ alkyl group and/or a halogen and/or an OR^{f} group and/or a NR^{g1}R^{g2} group; such as, for example, a 2-methyl-cyclopropyl group, a 2,2-dimethylcyclopropyl group, a 2,2-dimethylcyclobutyl group, a 3-hydroxycyclopentyl group, a 3-hydroxycyclohexylgroup, a 3-dimethylaminocyclobutyl group, a 3-dimethylaminocyclopentyl group or a 4-dimethylaminocyclohexyl group.

The term “cycloalkylalkyl” refers to cyclic ring-containing radicals containing in the range of about 3 up to 8 carbon atoms directly attached to the alkyl group which is then also attached to the main structure at any carbon from the alkyl group that results in the creation of a stable structure such as cyclopropylmethyl, cyclobutylethyl, and cyclopentylethyl.

The term “aryl” refers to aromatic radicals having in the range of 6 up to 14 carbon atoms such as phenyl, naphthyl, tetrahydronaphthyl, indanyl, biphenyl being optionally further substituted by an C₁-C₆ alkyl group and/or a halogen atom.

The term “arylalkyl” refers to an aryl group as defined herein directly bonded to an alkyl group as defined herein which is then attached to the main structure at any carbon from alkyl group that results in the creation of a stable structure at the rest of the molecule. e.g., —CH₂C₆H₅, —C₂H₅C₆H₅.

The term “heterocyclic ring” refers to a stable 3- to 15 membered ring radical which consists of carbon atoms and from one to five heteroatoms selected from the group consisting of nitrogen, phosphorus, oxygen and sulfur. For purposes of this invention, the heterocyclic ring radical may be a monocyclic, bicyclic or tricyclic ring system, which may include fused, bridged or spiro ring systems, and the nitrogen, phosphorus, carbon, oxygen or sulfur atoms in the heterocyclic ring radical is optionally oxidized to various oxidation states. In addition, the nitrogen atom is optionally quaternized and the ring radical may be partially or fully saturated (i.e., heteroaromatic or heteroaryl aromatic). Examples of such heterocyclic ring radicals include, but are not limited to, azetidinyl, acridinyl, benzodioxolyl, benzodioxanyl, benzofuranyl, carbazolyl, cinnolinyl, dioxotanyl, indolizinyl, naphthyridinyl, perhydroazepinyl, phenazinyl, phenothiazinyl, phenoxazinyl, phthalazil, pyridyl, pteridinyl, purinyl, quinazolinyl, quinoxatinyl, quinolinyl, isoquinolinyl, tetrazolyl, imidazolyl, tetrahydroisoindolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazinyl, pyrimidinyl, pyridazinyl, oxazolyl, oxazolinyl, oxazolidinyl, triazolyl, indanyl, isoxazolyl, isoxazolidinyl, morpholinyl, thiazotyl, thiazolinyl, thiazolidinyl; isothiazolyl, quinuctidinyl, isothiazolidinyl, indotyl, isoindolyl, indotinyl, isoindolinyl, octahydroindolyl, octahydroisoindolyl, quinolyl, isoquinolyl, decahydroisoquinotyl, benzimidazolyl, thiadiazolyl, benzopyranyl, benzothiazolyl, benzooxazolyl, furyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl.

The term “heterocycloalkyl” is to be understood as preferably meaning a C₃-C₁₀ cycloalkyl group, as defined supra, featuring the indicated number of ring atoms, wherein one or more ring atom(s) is (are) (a) heteroatom(s) such as NH, NR^{d3}, O, S, or (a) group(s) such as a C(O), S(O), S(O)₂, or, otherwise stated, in a Cₙ-cycloalkyl group, (wherein n is an integer of 3, 4, 5, 6, 7, 8, 9, or 10), one or more carbon atom(s) is (are) replaced by said heteroatom(s) or said group(s) to give such a Cₙ cycloheteroalkyl group; and also as meaning an unsaturated heterocycloalkyl group containing one or more double bonds in the C-backbone, wherein the linkage of said heterocyclotalkyl group to the rest of the molecule can be provided to the double or single bond; and also as meaning such a saturated or unsaturated heterocycloalkyl group being optionally substituted one or more times, independently of each other, with a C₁-C₆ alkyl group and/or a halogen and/or an OR^{f} group and/or a NR^{g1}R^{g2} group. Thus, said Cₙ cycloheteroalkyl group refers, for example, to a three-membered heterocycloalkyl, expressed as C₃-heterocycloalkyl, such as oxiranyl (C₃). Other examples of heterocycloalkyls are oxetanyl (C₄), aziridinyl (C₃), azetidinyl (C₄), tetrahydrofuranyl (C₅), pyrrolidinyl (C₅), morpholinyl (C₆), dithianyl (C₆), thiomorpholinyl (C₆), piperidinyl (C₆), tetrahydropyranyl (C₆), piperazinyl (C₆), trithianyl (C₆), homomorpholinyl (C₇), homopiperazinyl (C₇) and chinuclidinyl (C₈) said cycloheteroalkyl group refers also to, for example, 4-methylpiperazinyl, 3-methyl-4-methylpiperazine, 3-fluoro-4-methylpiperazine, 4-dimethylaminopiperidinyl, 4-methylaminopiperidinyl, 4-aminopiperidinyl, 3-dimethylaminopiperidinyl, 3-methylaminopiperidinyl, 3-aminopiperidinyl, 4-hydroxypiperidinyl, 3-hydroxypiperidinyl, 2-hydroxypiperidinyl, 4-methylpiperidinyl, 3-methylpiperidinyl, 3-dimethylaminopyrrolidinyl, 3-methylaminopyrrolidinyl, 3-aminopyrrolidinyl or methylmorpholinyl.

The term “heteroaryl” refers to a heterocyclic ring radical as defined herein which is aromatic being optionally further substituted by an C₁-C₆ alkyl group and/or a halogen atom. The heteroaryl ring radical may be attached to the main structure at any heteroatom or carbon atom that results in the creation of a stable structure.

The heterocyclic ring radical may be attached to the main structure at any heteroatom or carbon atom that results in the creation of a stable structure.

The term “heteroarylalkyl” refers to heteroaryl ring radical as defined herein directly bonded to alkyl group. The heteroarylalkyl radical may be attached to the main structure at any carbon atom from the alkyl group that results in the creation of a stable structure.

The term “heterocyclyl” refers to a heterocylic ring radical as defined herein. The heterocylyl ring radical may be attached to the main structure at any heteroatom or carbon atom that results in the creation of a stable structure.

The term “heterocyclylalkyl” refers to a heterocyclic ring radical as defined herein directly bonded to alkyl group. The heterocyctylalkyl radical may be attached to the main structure at carbon atom in the alkyl group that results in the creation of a stable structure.

The term “carbonyl” refers to an oxygen atom bound to a carbon atom of the molecule by a double bond.

The term “halogen” refers to radicals of fluorine, chlorine, bromine and iodine.

Where the plural form of the word compounds, salts, polymorphs, hydrates, solvates and the like, is used herein, this is taken to mean also a single compound, salt, polymorph, isomer, hydrate, solvate or the like.

The compounds of this invention may contain one or more asymmetric centers, depending upon the location and nature of the various substituents desired. Asymmetric carbon atoms may be present in the (R) or (S) configuration, resulting in racemic mixtures in the case of a single asymmetric center, and diastereomeric mixtures in the case of multiple asymmetric centers. In certain instances, asymmetry may also be present due to restricted rotation about a given bond, for example, the central bond adjoining two substituted aromatic rings of the specified compounds. Substituents on a ring may also be present in either cis or trans form. It is intended that all such configurations (including enantiomers and diastereomers), are included within the scope of the present invention. Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallization. The optically active bases or acids are then liberated from the separated diastereomeric salts. A different process for separation of optical isomers involves the use of chiral chromatography (e.g., chiral HPLC columns), with or without conventional derivitization, optimally chosen to maximize the separation of the enantiomers. Suitable chiral HPLC columns are manufactured by Diacel, e.g., Chiracel OD and Chiracel OJ among many others, all routinely selectable. Enzymatic separations, with or without derivitization, are also useful. The optically active compounds of this invention can likewise be obtained by chiral syntheses utilizing optically active starting materials.

The present invention also relates to useful forms of the compounds as disclosed herein, such as pharmaceutically acceptable salts, co-precipitates, metabolites, hydrates, solvates and prodrugs of all the compounds of examples. The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19. Pharmaceutically acceptable salts include those obtained by reacting the main compound, functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid and citric acid. Pharmaceutically acceptable salts also include those in which the main compound functions as an acid and is reacted with an appropriate base to form, e.g., sodium, potassium, calcium, magnesium, ammonium, and chorine salts. Those skilled in the art will further recognize that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

Representative salts of the compounds of this invention include the conventional non-toxic salts and the quaternary ammonium salts which are formed, for example, from inorganic or organic acids or bases by means well known in the art. For example, such acid addition salts include acetate, adipate, alginate, ascorbate, aspartate, benzoate, benzenesulfonate, bisulfate, butyrate, citrate, camphorate, camphorsulfonate, cinnamate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, itaconate, lactate, maleate, mandelate, methanesulfonate, 2-naphthalenesulfonate, nicotinate, nitrate, oxalate, pamoate, pectinate, persulfate, 3-phenylpropionate, picrate, pivalate, propionate, succinate, sulfonate, tartrate, thiocyanate, tosylate, and undecanoate.

Base salts include alkali metal salts such as potassium and sodium salts, alkaline earth metal salts such as calcium and magnesium salts, and ammonium salts with organic bases such as dicyclohexylamine and N-methyl-D-glucamine. Additionally, basic nitrogen containing groups may be quaternized with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

A solvate for the purpose of this invention is a complex of a solvent and a compound of the invention in the solid state. Exemplary solvates would include, but are not limited to, complexes of a compound of the invention with ethanol or methanol. Hydrates are a specific form of solvate wherein the solvent is water.

### Method(s) of Making the Compounds of the Invention

### General Preparative Methods

The particular process to be utilized in the preparation of the compounds used in this embodiment of the invention depends upon the specific compound desired. Such factors as the selection of the specific substituents play a role in the path to be followed in the preparation of the specific compounds of this invention. Those factors are readily recognized by one of ordinary skill in the art.

The compounds of the invention may be prepared by use of known chemical reactions and procedures. Nevertheless, the following general preparative methods are presented to aid the reader in synthesizing the compounds of the present invention, with more detailed particular examples being presented below in the experimental section describing the working examples.

The compounds of the invention can be made according to conventional chemical methods, and/or as disclosed below, from starting materials which are either commercially available or producible according to routine, conventional chemical methods. General methods for the preparation of the compounds are given below, and the preparation of representative compounds is specifically illustrated in examples.

Synthetic transformations that may be employed in the synthesis of compounds of this invention and in the synthesis of intermediates involved in the synthesis of compounds of this invention are known by or accessible to one skilled in the art. Collections of synthetic transformations may be found in compilations, such as:
J. March. Advanced Organic Chemistry, 4th ed.; John Wiley: New York (1992)
R. C. Larock. Comprehensive Organic Transformations, 2nd ed.; Wiley-VCH: New York (1999)
F. A. Carey; R. J. Sundberg. Advanced Organic Chemistry, 2nd ed.; Plenum Press: New York (1984)
T. W. Greene; P. G. M. Wuts. Protective Groups in Organic Synthesis, 3rd ed.; John Wiley: New York (1999)
L. S. Hegedus. Transition Metals in the Synthesis of Complex Organic Molecules, 2nd ed.; University Science Books: Mill Valley, Calif. (1994)
L. A. Paquette, Ed. The Encyclopedia of Reagents for Organic Synthesis; John Wiley: New York (1994)
A. R. Katritzky; 0. Meth-Cohn; C. W. Rees, Eds. Comprehensive Organic Functional Group Transformations; Pergamon Press: Oxford, UK (1995)
G. Wilkinson; F. G. A. Stone; E. W. Abel, Eds. Comprehensive Organometallic Chemistry; Pergamon Press: Oxford, UK (1982)
B. M. Trost; I. Fleming. Comprehensive Organic Synthesis; Pergamon Press: Oxford, UK (1991)
A. R. Katritzky; C. W. Rees Eds. Comprehensive Heterocylic Chemistry; Pergamon Press: Oxford, UK (1984)
A. R. Katritzky; C. W. Rees; E. F. V. Scriven, Eds. Comprehensive Heterocylic Chemistry II; Pergamon Press: Oxford, UK (1996)
C. Hansch; P. G. Sammes; J. B. Taylor, Eds. Comprehensive Medicinal Chemistry: Pergamon Press: Oxford, UK (1990).

In addition, recurring reviews of synthetic methodology and related topics include Organic Reactions; John Wiley: New York; Organic Syntheses; John Wiley: New York; Reagents for Organic Synthesis: John Wiley: New York; The Total Synthesis of Natural Products; John Wiley: New York; The Organic Chemistry of Drug Synthesis; John Wiley New York; Annual Reports in Organic Synthesis; Academic Press: San Diego Calif.; and Methoden der Organischen Chemie (Houben-Weyt); Thieme: Stuttgart, Germany. Furthermore, databases of synthetic transformations include Chemical Abstracts, which may be searched using either CAS OnLine or SciFinder, Handbuch der Organischen Chemie (Beilstein), which may be searched using SpotFire, and REACCS.

### Reaction Schemes:

The following schemes illustrate general synthetic routes to the compounds of general formula (I) of the invention and are not intended to be limiting. It needs to be understood that transformations generically described in the following paragraphs may be performed at different reaction temperatures and in different solvents depending upon, for example, the reactivity of reagents and their respective solubility characteristics. More specifically, certain transformations may require heating in a solvent of a suitable boiling point. In specific cases heating of reaction mixtures may be achieved by using a microwave oven. In certain cases additives such as, for example, bases, phase transfer catalysts or ionic liquids may be used to modify reaction conditions to improve reaction turnover order heating characteristics. It is obvious to the person skilled in the art that the order of transformations as exemplified in Schemes 1 to 8 can be modified in various ways. The order of transformations exemplified in Schemes 1 to 8 is therefore not intended to be limiting. In addition, interconversion of substituents, for example of residues R¹, R², R³, R⁵, R⁶, R^{6a}, R⁷ or R^{a} can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts in Protective Groups in Organic Synthesis, 3^{rd} edition, Wiley 1999).

Reaction Scheme 1 illustrates one general method for the preparation of the Formula (I) compounds. A 2,6-difluorophenyl derivative of Formula (II) carrying an electron-withdrawing R⁵ substituent is reacted with an aniline of Formula (III) and base to form the amine intermediate of Formula (IV). This intermediate is reacted with an alcohol R^{6a}OH [Formula (V) where X=O], a thiol R^{6a}SH [Formula (V) where X═S], or an amine R^{6a}NH₂ [Formula (V) where X═NH] to form a product of Formula (Ia). This compound is optionally liberated from its protecting group (acetal or Boc) using an acid such as HCl or TFA to form the final product of Formula (I).

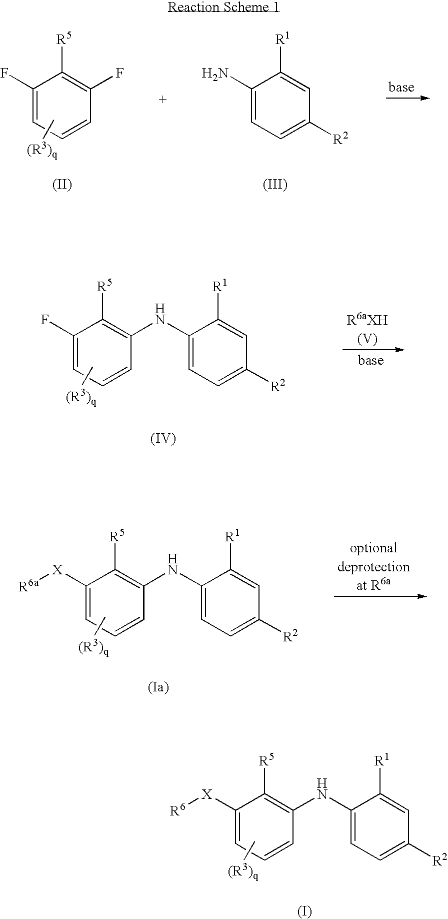

Scheme 1 General procedure for the preparation of compounds of the general Formula (I), wherein R¹, R², R³, R⁵, R⁶, X and q are as defined in the description and claims of this invention and R^{6a} stands for an optionally protected form of a R⁶ group, for example, for a R⁶ group carrying a Boc-protection group or an acetal.

Reaction Scheme 2 illustrates a further general method for the preparation of the Formula (I) compounds. A 2,6-difluorophenyl derivative of Formula (II) carrying an electron-withdrawing R⁵ substituent is reacted in the presence of a base with an alcohol R^{6a}OH [Formula (V) where X=O], a thiol R^{6a}SH [Formula (V) where X═S], or an amine R^{6a}NH₂ [Formula (V) where X═NH] to form an intermediate of Formula (VI). This intermediate is reacted with an aniline of Formula (III) in the presence of a base to form a product of Formula (Ia). This compound is optionally liberated from its protecting group (e.g. acetal or Boc) using an acid, for example hydrochloric acid or TFA, to form the final product of Formula (I).

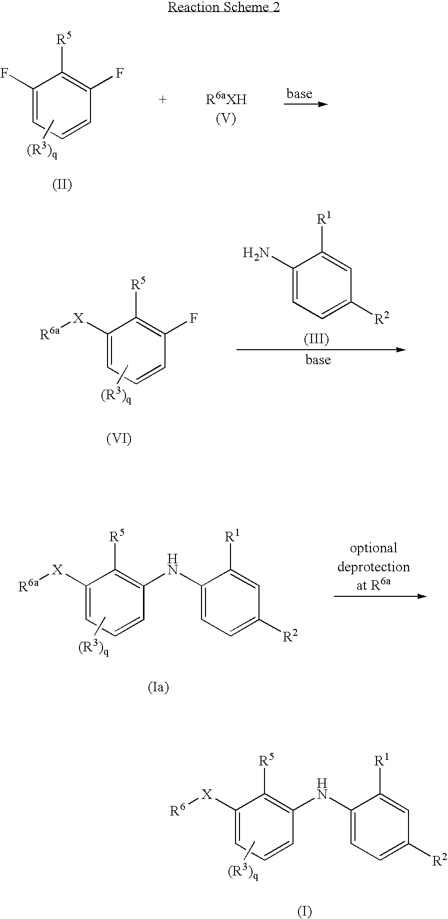

Scheme 2 General procedure for the preparation of compounds of the general Formula (I), wherein R¹, R², R³, R⁵, R⁶, X and q are as defined in the description and claims of this invention and R^{6a} stands for an optionally protected form of a R⁶ group, for example, for a R⁶ group carrying a Boc-protection group or an acetal.

Reaction Scheme 3 illustrates one further preferred general method for the preparation of the formula (I) compounds. A 2,6-difluorophenyl derivative of formula (II) carrying an electron-withdrawing R⁵ substituent is reacted in the presence of a base with an aniline of formula (III) to form a product of formula (IV). Protection of the aniline functionality yields a product of formula (VII), in which PG represents a suitable protecting group such as, for example, a tert-butoxycarbonyl (Boc) group, a benzyloxy carbonyl group or derivatives thereof or an acetyl group or derivatives thereof. Appropriate protecting group reagents and their introduction are well-known to the person skilled in the art (see for example T. W. Greene and P. G. M. Wuts in Protective Groups in Organic Synthesis, 3^{rd} edition, Wiley 1999). This product is subsequently reacted in the presence of a base with a compound of formula (V) to form product (VIII). This compound is optionally liberated from its protecting groups in a concerted or stepwise fashion using, for example, an acid, such as, for example, hydrochloric acid or TFA, or a base, such as, for example, sodium hydroxide, sodium ethanolate or lithium hydroxide, to form the final product of Formula (I). In a more specific application of this general method, the R⁵ group and the PG group in compounds of Formulae (VII) and (VIII) may form a 5- or 6-membered cycle. For example, in a case where the R⁵ group in Formula (IV) stands for a carboxylic acid, reaction with paraformaldehyde would lead to a benzoxazine which could be cleaved—after reaction with a R^{6a}XH group—by reaction with, for example, polymer bound glycerol and hydrochloric acid thereby providing a compound of Formula (Ia), in which R⁵ would stand for a carboxylic acid.

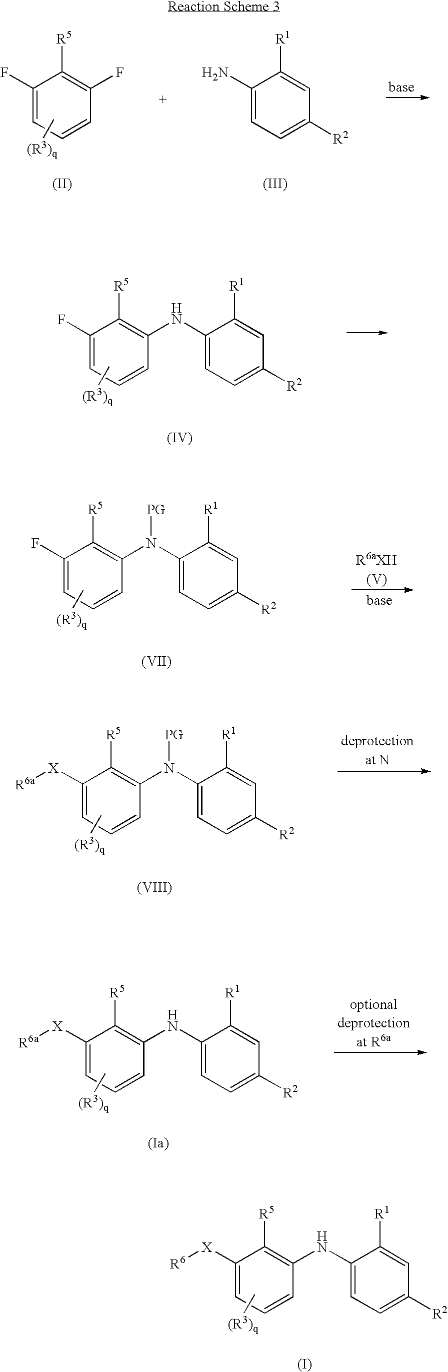

Scheme 3 General procedure for the preparation of compounds of the general Formula (I), wherein R¹, R², R³, R⁵, R⁶, X and q are as defined in the description and claims of this invention, R^{6a} stands for an optionally protected form of a R⁶ group, for example, for a R⁶ group carrying a Boc-protection group or an acetal, and PG stands for a suitable protecting groups such as, for example, a Boc group or a benzyloxycarbonyl group or derivatives thereof or an acetate or derivatives thereof.

Reaction Scheme 4 illustrates a more specific method for the preparation of the Formula (Id) compounds [Formula (I) where R⁵═C(O)NH₂]. A nitrile of Formula (Ib) [Formula (Ia) where R⁵═CN], prepared as described in Schemes 1 to 3, is transformed into the corresponding amide derivative of Formula Ic [Formula (Ia) where R⁵═C(O)NH₂]. Suitable conditions for this transformation include, but are not limited to, the treatment with hydrogen peroxide in the presence of a base. Compound (Ic) is optionally liberated from its protecting group (acetat or Boc) using an acid such as HCl or TFA to form the final product of Formula (Id).

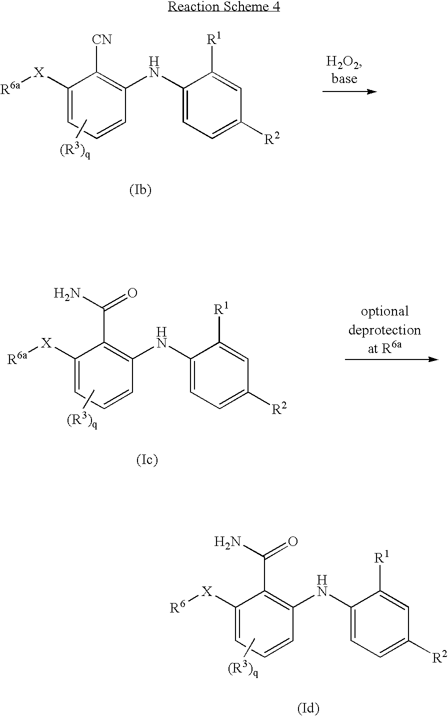

Scheme 4 More specific procedure for the preparation of compounds of the general Formula (Id), wherein R¹, R², R³, R⁶, X and q are as defined in the description and claims of this invention and R^{6a} stands for an optionally protected form of a R⁶ group, for example, for a R⁶ group carrying a Boc-protection group or an acetal.

Reaction Scheme 5 illustrates a general method for the preparation of the Formula (Ig) compounds [Formula (I) where R²=ethinyl]. An intermediate of Formula (Ie) [Formula (Ia) where R²=iodo], prepared as described in Schemes 1 to 4, is reacted with ethine in the presence of catalytic amounts of a Pd catalyst such as PdCl₂(PPh₃)₂, catalytic amounts of copper iodide, in the presence of a solvent such as DMF to form the corresponding alkyne derivative of Formula If [Formula (Ia) where R²=ethinyl]. Alternatively, mono-trialkylsilyl-protected acetylene such as for example, trimethylsilyl (TMS) acetylene, may be employed in a Sonogashira-type coupling under conditions as described above followed by cleavage of the trialkylsilyl group by treatment with, for example, tetrabutylammonium fluoride or potassium carbonate in methanol. Alternatively, by using tetrabutylammonium fluoride as base in the Sonogashira-type coupling, coupling of TMS acetylene and cleavage of the TMS-group can be achieved in a one pot transformation. Transition metal-catalyzed couplings of (hetero)aryl halides with alkynes and trialkylsilyl alkynes are well known to the person skilled in the art (see for example (a) Chinchilla, R.; Najera, C. Chem. Rev. 2007, 107, 874; (b) Negishi, E.-i., Anastasia, L. Chem. Rev. 2003, 103, 1979; see also: (c) Eur. J. Org. Chem. 2005, 20, 4256; (d) J. Org. Chem. 2006, 71, 2535 and references therein; (e) Chem. Commun. 2004, 17, 1934). Various palladium-catalyst/co-catalyst/ligand/base/solvent combinations have been published in the scientific Literature which allow a fine-tuning of the required reaction conditions in order to allow for a broad set of additional functional groups on both coupling partners (see references in the above cited reviews). Additionally, recently developed procedures employing e.g. zinc acetylides, alkynyl magnesium salts or alkynyl trifluoroborate salts further broaden the scope of this process. Compound (If) is optionally liberated from its protecting group (acetal or Boc) using an acid such as HCl or TFA to form the final product of Formula (Ig). Furthermore, the described procedures can be applied to further alkyne substrates, such as, for example, C1-C6 alkynes.

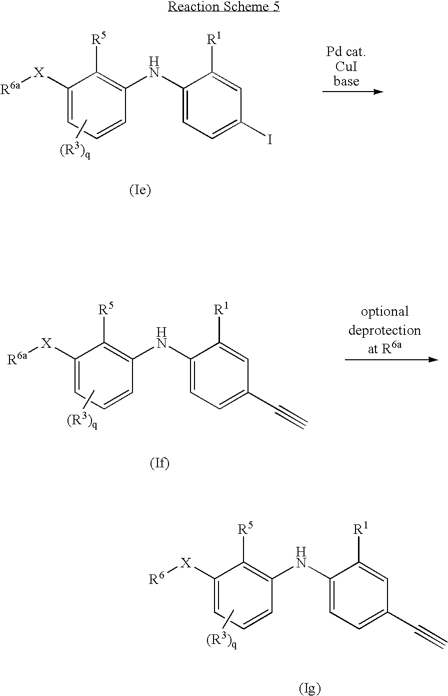

Scheme 5 General procedure for the preparation of compounds of the general Formula (Ig) by coupling of an iodide of general formula (Ie) is reacted with a suitable alkyne to yield a compound of Formula (If), wherein R¹, R², R³, R⁵, R⁶, X and q are as defined in the description and claims of this invention and R^{6a} stands for an optionally protected form of a R⁶ group, for example, for a R⁶ group carrying a Boc-protection group or an acetal.

Reaction Scheme 6 illustrates one general method for the preparation of the Formula (II) compounds [Formula (I) where R⁵═C(O)NHR⁷]. An intermediate of Formula Ic [Formula (Ia) where R⁵═C(O)NH₂], prepared as described in Schemes 1 to 5, is reacted with an alkylating reagent to form the corresponding N-alkyl amide derivative of Formula Ih [Formula (Ia) where R⁵═C(O)NHR⁷]. This compound is optionally liberated from its protecting group (acetal or Boc) using an acid such as HCl or TFA to form the final product of Formula (II).

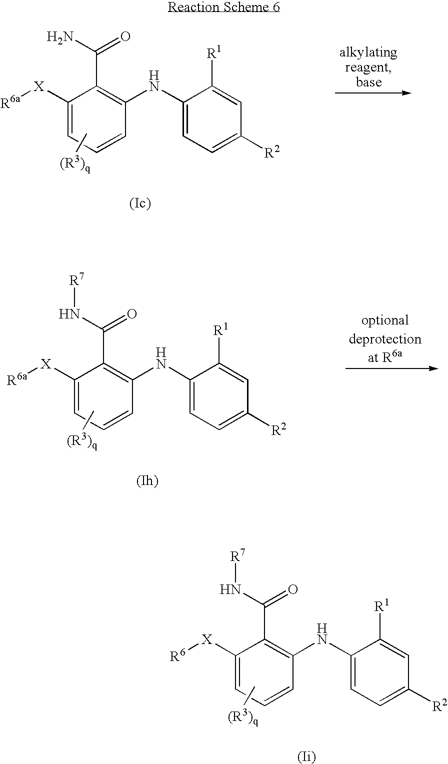

Scheme 6 General procedure for the preparation of compounds of the general Formula (II), wherein R¹, R², R³, R⁶, R⁷, X and q are as defined in the description and claims of this invention and R^{6a} stands for an optionally protected form of a R⁶ group, for example, for a R⁶ group carrying a Boc-protection group or an acetal.

Reaction Scheme 7 illustrates the general method for the preparation of the Formula (In) compounds. An intermediate of Formula (Im), prepared as described in Schemes 1 to 6, is reacted with a dihydroxylating agent such as, for example, osmiumtetroxide, optionally in the presence of a promoter such as, for example, DMAP and in a suitable solvent such as, for example, acetone, to form the corresponding bishydroxy derivative of Formula (In) as final compound. Similarly, analogs of compounds of Formula (Im), in which the double bond is further substituted with alkyl groups or part of a cycloalkenyl ring, can be applied to the described dihydroxylation conditions leading to analogs of compounds of Formula (In), in which the oxygenated carbon atoms carry additional alkyl groups. Alternatively, asymmetric dihydroxylation conditions as known to the person skilled in the art can be employed to achieve the general transformation shown in Scheme 7 in an enantioselective fashion.

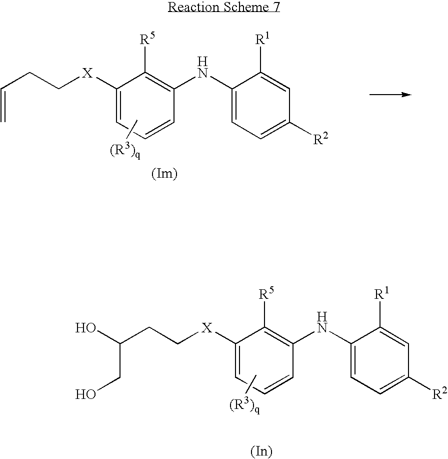

Scheme 7 General procedure for the preparation of compounds of the general Formula (In), wherein R¹, R², R³, R⁵, X and q are as defined in the description and claims of this invention.

Reaction Scheme 8 illustrates one additional specific method for the preparation of the Formula (It) compounds. An intermediate of Formula (Ir), prepared by procedures described above, is transformed into the corresponding methansulfonate (mesylate) by reaction with, for example, methansulfonyl chloride, optionally in the presence of a base. Subsequently this mesylate of Formula (Ir) is reacted either in situ or after isolation with an amine of general formula (IX) to afford a compound of Formula (It). Other ways of activating an alcohol for a subsequent nucleophilic substitution reaction are known to the person skilled in the art, such as, for example, transformation into a para-toluene sulfonate (tosylate) or a nitro-phenylsulfonate.

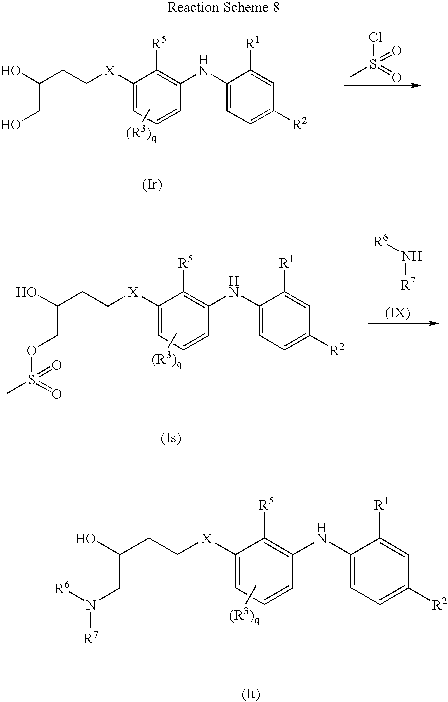

Scheme 8 General procedure for the preparation of compounds of the general Formula (It), wherein R¹, R², R³, R⁵, R⁶, R⁷, X and q are as defined in the description and claims of this invention.

### Pharmaceutical Compositions of the Compounds of the Invention

This invention also relates to pharmaceutical compositions containing one or more compounds of the present invention. These compositions can be utilized to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatin, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, coloring agents, and flavoring agents such as peppermint, oil of wintergreen, or cherry flavoring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavoring and coloring agents.

The compounds of this invention may also be administered parenterally, that is, subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables. A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, issued Jun. 11, 1991, incorporated herein by reference). Such patches may be constructed for continuous, pulsatile, or on demand delivery of pharmaceutical agents.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized. Such ingredients and procedures include those described in the following references, each of which is incorporated herein by reference: Powell, M. F. et al, “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al, “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);
alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);
adsorbents (examples include but are not limited to powdered cellulose and activated charcoal);
aerosol propellants (examples include but are not limited to carbon dioxide, CCl₂F₂, F₂ClC—CClF₂ and CClF₃)
air displacement agents (examples include but are not limited to nitrogen and argon);
antifungal preservatives (examples include but are not limited to benzoic acid, butylparaben, ethylparaben, methylparaben, propylparaben, sodium benzoate);
antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);
antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);
binding materials (examples include but are not limited to block polymers, natural and synthetic rubber, polyacrylates, polyurethanes, silicones, polysiloxanes and styrene-butadiene copolymers);
buffering agents (examples include but are not limited to potassium metaphosphate, dipotassium phosphate, sodium acetate, sodium citrate anhydrous and sodium citrate dihydrate)
carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection)
chelating agents (examples include but are not limited to edetate disodium and edetic acid)
colorants (examples include but are not limited to FD&TC Red No. 3, FD&TC Red No. 20, FD&C Yellow No. 6, FD&C Blue No. 2, D&C Green No. 5, D&C Orange No. 5, D&C Red No. 8, caramel and ferric oxide red);
clarifying agents (examples include but are not limited to bentonite);
emulsifying agents (examples include but are not limited to acacia, cetomacrogot, cetyl alcohol, glyceryl monostearate, lecithin, sorbitan monooleate, polyoxyethylene 50 monostearate);
encapsulating agents (examples include but are not limited to gelatin and cellulose acetate phthalate)
flavorants (examples include but are not Limited to anise oil, cinnamon oil, cocoa, menthol, orange oil, peppermint oil and vanillin);
humectants (examples include but are not limited to glycerol, propylene glycol and sorbitol);
levigating agents (examples include but are not limited to mineral oil and glycerin);
oils (examples include but are not limited to arachis oil, mineral oil, olive oil, peanut oil, sesame oil and vegetable oil);
ointment bases (examples include but are not limited to lanolin, hydrophilic ointment, polyethylene glycol ointment, petrolatum, hydrophilic petrolatum, white ointment, yellow ointment, and rose water ointment);
penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas)
plasticizers (examples include but are not limited to diethyl phthalate and glycerol)
solvents (examples include but are not limited to ethanol, corn oil, cottonseed oil, glycerol, isopropanol, mineral oil, oleic acid, peanut oil, purified water, water for injection, sterile water for injection and sterile water for irrigation);
stiffening agents (examples include but are not limited to cetyl alcohol, cetyl esters wax, microcrystalline wax, paraffin, stearyl alcohol, white wax and yellow wax);
suppository bases (examples include but are not limited to cocoa butter and polyethylene glycols (mixtures));
surfactants (examples include but are not limited to benzalkonium chloride, nonoxynol 10, oxtoxynol 9, polysorbate 80, sodium lauryl sulfate and sorbitan mono-palmitate);
suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);
sweetening agents (examples include but are not limited to aspartame, dextrose, glycerol, mannitol, propylene glycol, saccharin sodium, sorbitol and sucrose);
tablet anti-adherents (examples include but are not limited to magnesium stearate and talc);
tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);
tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);
tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);
tablet direct compression excipients (examples include but are not limited to dibasic calcium phosphate);
tablet disintegrants (examples include but are not limited to alginic acid, carboxymethylcellulose calcium, microcrystalline cellulose, polacrillin potassium, cross-linked polyvinylpyrrolidone, sodium alginate, sodium starch glycollate and starch);
tablet glidants (examples include but are not limited to colloidal silica, corn starch and talc);
tablet lubricants (examples include but are not limited to calcium stearate, magnesium stearate, mineral oil, stearic acid and zinc stearate);
tablet/capsule opaquants (examples include but are not limited to titanium dioxide);
tablet polishing agents (examples include but are not limited to carnuba wax and white wax);
thickening agents (examples include but are not limited to beeswax, cetyl alcohol and paraffin);
tonicity agents (examples include but are not limited to dextrose and sodium chloride);
viscosity increasing agents (examples include but are not limited to alginic acid, bentonite, carbomers, carboxymethylcellulose sodium, methylcellulose, polyvinyl pyrrolidone, sodium alginate and tragacanth); and
wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile IV Solution: A 5 mg/mL solution of the desired compound of this invention can be made using sterile, injectable water, and the pH is adjusted if necessary. The solution is diluted for administration to 1-2 mg/mL with sterile 5% dextrose and is administered as an IV infusion over about 60 minutes.
Lyophilized powder for IV administration: A sterile preparation can be prepared with (i) 100-1000 mg of the desired compound of this invention as a lypholized powder, (ii) 32-327 mg/mL sodium citrate, and (iii) 300-3000 mg Dextran 40. The formulation is reconstituted with sterile, injectable saline or dextrose 5% to a concentration of 10 to 20 mg/mL, which is further diluted with saline or dextrose 5% to 0.2-0.4 mg/mL, and is administered either IV bolus or by IV infusion over 15-60 minutes.
Intramuscular suspension: The following solution or suspension can be prepared, for intramuscular injection:
50 mg/mL of the desired, water-insoluble compound of this invention
5 mg/mL sodium carboxymethylcellulose
4 mg/mL TWEEN 80
9 mg/mL sodium chloride
9 mg/mL benzyl alcohol
Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.
Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.
Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.
Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### Method of Treating Hyper-Proliferative Disorders

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not Limited to small-cell and non-small-cell Lung carcinoma, as well as bronchial adenoma and pleuropulmonary blastoma.

Examples of brain cancers include, but are not Limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not Limited to anal, colon, colorectal, esophageal, gallbladder, gastric, pancreatic, rectal, small-intestine, and salivary gland cancers.

Tumors of the urinary tract include, but are not Limited to bladder, penile, kidney, renal pelvis, ureter, urethral and human papillary renal cancers.

Eye cancers include, but are not limited to intraocular melanoma and retinoblastoma.

Examples of Liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to Laryngeal, hypopharyngeal, nasopharyngeal, oropharyngeal cancer, lip and oral cavity cancer and squamous cell. Lymphomas include, but are not limited to AIDS-related lymphoma, non-Hodgkin's lymphoma, cutaneous T-cell lymphoma, Burkitt lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

### Methods of Treating Kinase Disorders

The present invention also provides methods for the treatment of disorders associated with aberrant mitogen extracellular kinase activity, including, but not limited to stroke, heart failure, hepatomegaly, cardiomegaly, diabetes, Alzheimer's disease, cystic fibrosis, symptoms of xenograft rejections, septic shock or asthma.

Effective amounts of compounds of the present invention can be used to treat such disorders, including those diseases (e.g., cancer) mentioned in the Background section above. Nonetheless, such cancers and other diseases can be treated with compounds of the present invention, regardless of the mechanism of action and/or the relationship between the kinase and the disorder.

The phrase “aberrant kinase activity” or “aberrant tyrosine kinase activity,” includes any abnormal expression or activity of the gene encoding the kinase or of the polypeptide it encodes. Examples of such aberrant activity, include, but are not limited to, over-expression of the gene or polypeptide; gene amplification; mutations which produce constitutively-active or hyperactive kinase activity; gene mutations, deletions, substitutions, additions, etc.

The present invention also provides for methods of inhibiting a kinase activity, especially of mitogen extracellular kinase, comprising administering an effective amount of a compound of the present invention, including salts, polymorphs, metabolites, hydrates, solvates, prodrugs (e.g.: esters) thereof, and diastereoisomeric forms thereof. Kinase activity can be inhibited in cells (e.g., in vitro), or in the cells of a mammalian subject, especially a human patient in need of treatment.

### Methods of Treating Angiogenic Disorders

The present invention also provides methods of treating disorders and diseases associated with excessive and/or abnormal angiogenesis.

Inappropriate and ectopic expression of angiogenesis can be deleterious to an organism. A number of pathological conditions are associated with the growth of extraneous blood vessels. These include, e.g., diabetic retinopathy, ischemic retinal-vein occlusion, and retinopathy of prematurity (Aiello et al. New Engl. J. Med. 1994, 331, 1480; Peer et al. Lab. Invest. 1995, 72, 638), age-related macular degeneration (AMD; see, Lopez et al. Invest. Opththalmol. Vis. Sci. 1996, 37, 855), neovascular glaucoma, psoriasis, retrolental fibroplasias, angiofibroma, inflammation, rheumatoid arthritis (R^{A}), restenosis, in-stent restenosis, vascular graft restenosis, etc. In addition, the increased blood supply associated with cancerous and neoplastic tissue, encourages growth, leading to rapid tumor enlargement and metastasis. Moreover, the growth of new blood and lymph vessels in a tumor provides an escape route for renegade cells, encouraging metastasis and the consequence spread of the cancer. Thus, compounds of the present invention can be utilized to treat and/or prevent any of the aforementioned angiogenesis disorders, e.g., by inhibiting and/or reducing blood vessel formation; by inhibiting, blocking, reducing, decreasing, etc. endothelial cell proliferation or other types involved in angiogenesis, as well as causing cell death or apoptosis of such cell types.

### Dose and Administration

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

### Combination Therapies

The compounds of this invention can be administered as the sole pharmaceutical agent or in combination with one or more other pharmaceutical agents where the combination causes no unacceptable adverse effects. For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

The additional pharmaceutical agent can be aldesleukin, alendronic acid, alfaferone, alitretinoin, allopurinol, aloprim, aloxi, altretamine, aminoglutethimide, amifostine, amrubicin, amsacrine, anastrozole, anzmet, aranesp, arglabin, arsenic trioxide, aromasin, 5-azacytidine, azathioprine, BCG or tice BCG, bestatin, betamethasone acetate, betamethasone sodium phosphate, bexarotene, bleomycin sulfate, broxuridine, bortezomib, busulfan, calcitonin, campath, capecitabine, carboplatin, casodex, cefesone, celmoleukin, cerubidine, chlorambucil, cisplatin, cladribine, cladribine, clodronic acid, cyclophosphamide, cytarabine, dacarbazine, dactinomycin, DaunoXome, decadron, decadron phosphate, delestrogen, denileukin diftitox, depo-medrol, deslorelin, dexrazoxane, diethylstilbestrol, diflucan, docetaxel, doxifluridine, doxorubicin, dronabinol, DW-166HC, eligard, elitek, ellence, emend, epirubicin, epoetin alfa, epogen, eptaplatin, ergamisol, estrace, estradiol, estramustine phosphate sodium, ethinyl estradiol, ethyol, etidronic acid, etopophos, etoposide, fadrozole, farston, filgrastim, finasteride, fligrastim, floxuridine, fluconazole, fludarabine, 5-fluorodeoxyuridine monophosphate, 5-fluorouracil (5-FU), fluoxymesterone, flutamide, formestane, fosteabine, fotemustine, fulvestrant, gammagard, gemcitabine, gemtuzumab, gleevec, gliadel, goserelin, granisetron HCl, histrelin, hycamtin, hydrocortone, eyrthro-hydroxynonyladenine, hydroxyurea, ibritumomab tiuxetan, idarubicin, ifosfamide, interferon alpha, interferon-alpha 2, interferon alfa-2A, interferon alfa-2B, interferon alfa-n1, interferon alfa-n3, interferon beta, interferon gamma-1a, interleukin-2, intron A, iressa, irinotecan, kytrit, lentinan sulphate, letrozole, leucovorin, leuprolide, leuprolide acetate, levamisole, levofolinic acid calcium salt, levothroid, levoxyl, lomustine, lonidamine, marinot, mechlorethamine, mecobalamin, medroxyprogesterone acetate, megestrol acetate, melphatan, menest, 6-mercaptopurine, Mesna, methotrexate, metvix, miltefosine, minocycline, mitomycin C, mitotane, mitoxantrone, Modrenal, Myocet, nedaplatin, neulasta, neumega, neupogen, nilutamide, nolvadex, NSC-631570, OCT-43, octreotide, ondansetron HCl, orapred, oxaliplatin, paclitaxel, pediapred, pegaspargase, Pegasys, pentostatin, picibanil, pilocarpine HCl, pirarubicin, plicamycin, porfimer sodium, prednimustine, prednisolone, prednisone, premarin, procarbazine, procrit, raltitrexed, rebif, rhenium-186 etidronate, rituximab, roferon-A, romurtide, salagen, sandostatin, sargramostim, semustine, sizofuran, sobuzoxane, solu-medrol, sparfosic acid, stem-cell therapy, streptozocin, strontium-89 chloride, synthroid, tamoxifen, tamsulosin, tasonermin, tastolactone, taxotere, teceleukin, temozolomide, teniposide, testosterone propionate, testred, thioguanine, thiotepa, thyrotropin, tiludronic acid, topotecan, toremifene, tositumomab, trastuzumab, treosulfan, tretinoin, trexall, trimethylmelamine, trimetrexate, triptorelin acetate, triptorelin pamoate, UFT, uridine, valrubicin, vesnarinone, vinblastine, vincristine, vindesine, vinorelbine, virulizin, zinecard, zinostatin stimalamer, zofran, ABI-007, acolbifene, actimmune, affinitak, aminopterin, arzoxifene, asoprisnil, atamestane, atrasentan, sorafenib, avastin, CCI-779, CDC-501, celebrex, cetuximab, crisnatol, cyproterone acetate, decitabine, DN-101, doxorubicin-MTC, dSLIM, dutasteride, edotecarin, eflornithine, exatecan, fenretinide, histamine dihydrochloride, histrelin hydrogel implant, holmium-166 DOTMP, ibandronic acid, interferon gamma, intron-PEG, ixabepilone, keyhole Limpet hemocyanin, L-651582, lanreotide, lasofoxifene, libra, lonafarnib, miproxifene, minodronate, MS-209, liposomal MTP-PE, MX-6, nafarelin, nemorubicin, neovastat, nolatrexed, oblimersen, onco-TCS, osidem, pactitaxel polyglutamate, pamidronate disodium, PN-401, QS-21, quazepam, R-1549, raloxifene, ranpirnase, 13-cis-retinoic acid, satraplatin, seocalcitol, T-138067, tarceva, taxoprexin, thymosin alpha 1, tiazofurine, tipifarnib, tirapazamine, TLK-286, toremifene, TransMID-107R, valspodar, vapreotide, vatalanib, verteporfin, vinflunine, Z-100, zoledronic acid or combinations thereof.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Motinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine mono-phosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethyl-melamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon alpha., beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, deniteukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, labetuzumab, alpha-particle-emitting radioisotope-linked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, atemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:

(1) yield better efficacy in reducing the growth of a tumor or even eliminate the tumor as compared to administration of either agent atone,
(2) provide for the administration of lesser amounts of the administered chemo-therapeutic agents,
   (3) provide for a chemotherapeutic treatment that is well tolerated in the patient with fewer deleterious pharmacological complications than observed with single agent chemotherapies and certain other combined therapies,
   (4) provide for treating a broader spectrum of different cancer types in mammals, especially humans,
   (5) provide for a higher response rate among treated patients,
   (6) provide for a longer survival time among treated patients compared to standard chemotherapy treatments,
   (7) provide a longer time for tumor progression, and/or
   (8) yield efficacy and tolerability results at least as good as those of the agents used alone, compared to known instances where other cancer agent combinations produce antagonistic effects.

### Methods of Sensitizing Cells to Radiation

In a distinct embodiment of the present invention, a compound of the present invention may be used to sensitize a cell to radiation. That is, treatment of a cell with a compound of the present invention prior to radiation treatment of the cell renders the cell more susceptible to DNA damage and cell death than the cell would be in the absence of any treatment with a compound of the invention. In one aspect, the cell is treated with at Least one compound of the invention.

Thus, the present invention also provides a method of killing a cell, wherein a cell is administered one or more compounds of the invention in combination with conventional radiation therapy.

The present invention also provides a method of rendering a cell more susceptible to cell death, wherein the cell is treated one or more compounds of the invention prior to the treatment of the cell to cause or induce cell death. In one aspect, after the cell is treated with one or more compounds of the invention, the cell is treated with at least one compound, or at least one method, or a combination thereof, in order to cause DNA damage for the purpose of inhibiting the function of the normal cell or killing the cell.

In one embodiment, a cell is killed by treating the cell with at least one DNA damaging agent. That is, after treating a cell with one or more compounds of the invention to sensitize the cell to cell death, the cell is treated with at least one DNA damaging agent to kill the cell. DNA damaging agents useful in the present invention include, but are not limited to, chemotherapeutic agents (eg., cisplatinum), ionizing radiation (X-rays, ultraviolet radiation), carcinogenic agents, and mutagenic agents.

In another embodiment, a cell is killed by treating the cell with at least one method to cause or induce DNA damage. Such methods include, but are not limited to, activation of a cell signaling pathway that results in DNA damage when the pathway is activated, inhibiting of a cell signaling pathway that results in DNA damage when the pathway is inhibited, and inducing a biochemical change in a cell, wherein the change results in DNA damage. By way of a non-limiting example, a DNA repair pathway in a cell can be inhibited, thereby preventing the repair of DNA damage and resulting in an abnormal accumulation of DNA damage in a cell.

In one aspect of the invention, a compound of the invention is administered to a cell prior to the radiation or orther induction of DNA damage in the cell. In another aspect of the invention, a compound of the invention is administered to a cell concomitantly with the radiation or orther induction of DNA damage in the cell. In yet another aspect of the invention, a compound of the invention is administered to a cell immediately after radiation or orther induction of DNA damage in the cell has begun.

In another aspect, the cell is in vitro. In another embodiment, the cell is in vivo.

### EXPERIMENTAL DETAILS AND GENERAL PROCESSES

### Abbreviations and Acronyms

A comprehensive list of the abbreviations used by organic chemists of ordinary skill in the art appears in The ACS Style Guide (third edition) or the Guidelines for Authors for the Journal of Organic Chemistry. The abbreviations contained in said lists, and all abbreviations utilized by organic chemists of ordinary skill in the art are hereby incorporated by reference. For purposes of this invention, the chemical elements are identified in accordance with the Periodic Table of the Elements, CAS version, Handbook of Chemistry and Physics, 67th Ed., 1986-87.

More specifically, when the following abbreviations are used throughout this disclosure, they have the following meanings:

Ac₂O acetic anhydride
ACN acetonitrile
AcO (or OAc) acetate
anhyd anhydrous
aq aqueous
Ar aryl
atm atmosphere
ATP adenosine triphosphate
b.i.d. twice a day
Biotage silica gel chromatographic system, Biotage Inc.
Bn benzyl
bp boiling point
Bz benzoyl
BOC tert-butoxycarbonyl
n-BuOH n-butanol
t-BuOH tert-butanol
t-BuOK potassium tert-butoxide
calcd calculated
Cbz carbobenzyloxy
CDI carbonyl diimidazole
CD₃OD methanol-d₄
Celite® diatomaceous earth filter agent, Cetite Corp.
CI-MS chemical ionization mass spectroscopy
¹³C NMR carbon-13 nuclear magnetic resonance
conc concentrated
DCC dicyclohexylcarbodiimide
DCE dichloroethane
DCM dichloromethane
dec decomposition
DIBAL diisobutylaluminum hydroxide

### DMAP 4-(N,N-dimethylamino)pyidine

DME 1,2-dimethoxyethane

### DMF N,N-dimethylformamide

DMSO dimethylsulfoxide
DTT dithiothreitol
E entgegen (configuration)
e.g. for example
EI electron impact
ELSD evaporative Light scattering detector
eq equivalent
ERK extracellutar signal-regulated kinase
ESI electrospray ionisation
ES-MS electrospray mass spectroscopy
et al. and others
EtOAc ethyl acetate
EtOH ethanol (100%)
EtSH ethanethiol
Et₂O diethyl ether
Et₃N triethylamine
GC gas chromatography
GC-MS gas chromatography-mass spectroscopy
h hour, hours
¹H NMR proton nuclear magnetic resonance
HCl hydrochloric acid
HEPES 4-(2-hydroxyethyl)-1-piperazineethanesulfonic acid
Hex hexane
HMPA hexamethylphosphoramide
HMPT hexamethylphosphoric triamide
HPLC high performance liquid chromatography
IC₅₀ drug concentration required for 50% inhibition
i.e. that is
insol insoluble
IPA isopropylamine
IR infrared
J coupling constant (NMR spectroscopy)
LAH lithium aluminum hydride
LC liquid chromatography
LC-MS liquid chromatography-mass spectrometry
LDA lithium diisopropylamide
MAPK mitogen-activated protein kinase
MeCN acetonitrile
MEK MAPK/ERK kinase
MHz megahertz
min minute, minutes
μL microliter
mL milliliter
μM micromolar
mp melting point
MS mass spectrum, mass spectrometry
Ms methanesulfonyl
m/z mass-to-charge ratio

### NBS N-bromosuccinimide

nM nanomolar
NMM 4-methylmorpholine
obsd observed
p page
PBS phosphate buffered saline
pp pages
PdCl₂dppf [1,1′-bis(diphenylphosphino)ferrocene]dichloropalladium(II)
Pd(OAc)₂ palladium acetate
pH negative logarithm of hydrogen ion concentration
pK negative logarithm of equilibrium constant
pKₐ negative logarithm of equilibrium constant for association
PS-DIEA polystyrene-bound diisopropylethylamine
q quartet (nmr)
qt quintet (nmr)
R^{f} retention factor (TLC)
RT retention time (HPLC)
rt room temperature
TBAF tetra-n-butylammonium fluoride
TBST tris buffered saline with tween
TEA triethylamine
THF tetrahydrofuran
TFA trifluoroacetic acid
TFFH fluoro-N,N,N′,N′-tetramethylformamidinium hexafluorophosphate
TLC thin layer chromatography
TMAD N,N, N′,N′-tetramethylethylenediamine
TMSCl trimethylsilyl chloride
Ts p-toluenesulfonyl
V/V volume per volume
w/v weight per volume
w/w weight per weight
z zusammen (configuration)

The percentage yields reported in the following examples are based on the starting component that was used in the lowest molar amount. Air and moisture sensitive liquids and solutions were transferred via syringe or cannula, and introduced into reaction vessels through rubber septa. Commercial grade reagents and solvents were used without further purification. The term “concentrated under reduced pressure” refers to use of a Buchi rotary evaporator at a minimum pressure of approximately 15 mm of Hg. All temperatures are reported uncorrected in degrees Celsius (° C.). Thin layer chromatography (TLC) was performed on pre-coated glass-backed silica gel 60 A F-254 250 μm plates.

The structures of compounds of this invention were confirmed using one or more of the following procedures.

### NMR

NMR spectra were acquired for each compound and were consistent with the structures shown.

Routine one-dimensional NMR spectroscopy was performed on 400 MHz Varian® Mercury-plus spectrometers. The samples were dissolved in deuterated solvents. Chemical shifts were recorded on the ppm scale and were referenced to the appropriate solvent signals, such as 2.49 ppm for DMSO-d⁶, 1.93 ppm for CD₃CN, 3.30 ppm for CD₃OD, 5.32 ppm for CD₂Cl₂ and 7.26 ppm for CDCl₃ for ¹H spectra.

### GC/MS

Electron impact mass spectra (EI-MS) were obtained with a Hewlett Packard 5973 mass spectrometer equipped Hewlett Packard 6890 Gas Chromatograph with a J&W HP-5 column (0.25 uM coating; 30 m×0.32 mm). The ion source was maintained at 250° C. and spectra were scanned from 50-550 amu at 0.34 sec per scan.

LC/MS Unless otherwise noted, all retention times are obtained from the LC/MS and correspond to the molecular ion. High pressure liquid chromatography-electrospray mass spectra (LC/MS) were obtained using a Hewlett-Packard 1100 HPLC equipped with a quaternary pump, a variable wavelength detector set at 254 nm, a Waters Sunfire C18 column (2.1×30 mm, 3.5 □m), a Gilson autosampler and a Finnigan LCQ ion trap mass spectrometer with electrospray ionization. Spectra were scanned from 120-1200 amu using a variable ion time according to the number of ions in the source. The eluents were A: 2% acetonitrile in water with 0.02% TFA, and B: 2% water in acetonirite with 0.018% TFA. Gradient elution from 10% B to 95% B over 3.5 minutes at a flow rate of 1.0 mL/min was used with an initial hold of 0.5 minutes and a final hold at 95% B of 0.5 minutes. Total run time was 6.5 minutes.

### Preparative HPLC:

Preparative HPLC was carried out in reversed phase mode using a Gilson HPLC system equipped with two Gilson 322 pumps, a Gilson 215 Autosampler, a Gilson diode array detector, and a C-18 column (e.g. YMC Pro 20×150 mm, 120 A). Gradient elution was used with solvent A as water with 0.1% TFA, and solvent B as acetonitrile with 0.1% TFA. Following injection onto the column as a solution, the compound was typically eluted with a mixed solvent gradient, such as 10-90% Solvent B in Solvent A over 15 minutes with flow rate of 25 mL/min. The fraction(s) containing the desired product were collected by UV monitoring at 254 or 220 nm.

### Preparative MPLC:

Preparative medium pressure liquid chromatography (MPLC) was carried out by standard silica get “flash chromatography” techniques (e.g., Still, W. C. et al. J. Org. Chem. 1978, 43, 2923-5), or by using silica get cartridges and devices such as the Combiflash and Biotage Flash systems. A variety of eluting solvents were used, as described in the experimental protocols.

In order that this invention may be better understood, the following examples are set forth. These examples are for the purpose of illustration only, and are not to be construed as limiting the scope of the invention in any manner. All publications mentioned herein are incorporated by reference in their entirety.

### Example 1.1

### 5-Fluoro-3-[(2-fluoro-4-iodophenyl)amino]-2-nitrophenoxybutane-1,2-diol

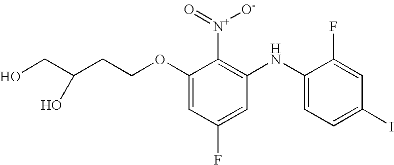

Step 1. Preparation of 3,5-difluoro-N-(2-fluoro-4-iodophenyl)-2-nitroaniline

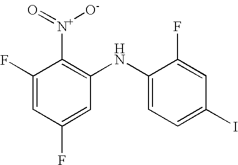

To the solution of 2-fluoro-4-iodoaniline (1.19 g, 5 mmol) in dry THF (10 mL) was added potassium tert-butoxide (617 mg, 5.50 mmol), and the mixture was stirred for 10 min, followed by addition of 1,3,5-trifluoro-2-nitrobenzene (885 mg, 5.00 mmol). The mixture was stirred for 30 min and then quenched with 5% aq acetic acid (30 mL). The mixture was extracted with EtOAc, and the combined organic layers were dried over sodium sulfate. After removal of the solvent under reduced pressure, the residue was purified by preparative TLC (DCM/methanol=15:1) to give the product (540 mg, 27%). ES/MS m/z 392.9 (M−H⁺); HPLC RT (min) 5.37.

Step 2. Preparation of 3-[2-(2,2-dimethyl-1,3-dioxolan-4-yl)ethoxy]-5-fluoro-N-(2-fluoro-4-iodophenyl)-2-nitroaniline

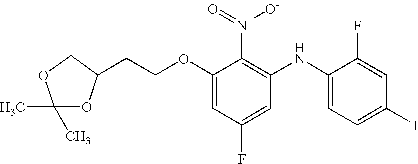

To the solution of 2-(2,2-dimethyl-1,3-dioxolan-4-yl)ethanol (44.5 mg, 0.304 mmol) in anhydrous THF (3 mL) was added sodium hydride (60%, 12.2 mg, 0.304 mmol), and the mixture was stirred for 10 min, followed by the addition of 3,5-difluoro-N-(2-fluoro-4-iodophenyl)-2-nitroaniline (100 mg, 0.254 mmol). The mixture was stirred for 30 min and then quenched with 5% aq acetic acid (10 mL). The mixture was extracted with EtOAc, and the combined organic layers were dried over sodium sulfate. After removal of the solvent, the crude product was purified by preparative TLC (DCM/methanol=15:1) to give the product (78 mg, 59%). ES/MS m/z 520.8 (MH⁺); HPLC RT (min) 5.57.

Step 3. Preparation of 4-{5-fluoro-3-[(2-fluoro-4-iodophenyl)amino]-2-nitrophenoxy}butane-1,2-diol

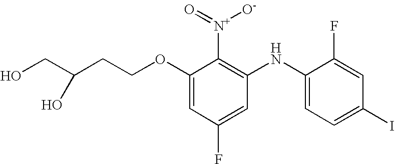

To a solution of 3-[2-(2,2-dimethyl-1,3-dioxolan-4-yl)ethoxy]-5-fluoro-N-(2-fluoro-4-iodophenyl)-2-nitroaniline (65.0 mg, 0.125 mmol) in acetonitrile (1.5 mL), was added conc. HCl (0.1 mL), the mixture was stirred at rt for 1 h. The reaction was quenched with 5% aq sodium bicarbonate. The mixture was extracted with EtOAc, and the combined organic layers were dried over sodium sulfate. The solvent was evaporated. The crude product was purified by preparative TLC (DCM/methanol=6:1) to give 46 mg (77%) product. ¹H NMR (400 MHz, CD₃OD), 7.58 (d, 1H), 7.52 (d, 1H), 7.09 (t, 1H), 6.52 (d, 1H), 6.23 (d, 1H), 4.21-4.26 (m, 2H), 3.83-3.87 (m, 1H), 3.47-3.56 (m, 2H), 1.99-2.02 (m, 1H), 1.76-1.82 (m, 1H). ES/MS m/z 480.9 (MH⁺); HPLC RT (min) 4.93.

### Example 1.2

### 5-Fluoro-N-(2-fluoro-4-iodophenyl)-2-nitro-3-(2-piperidin-4-ylethoxy)aniline

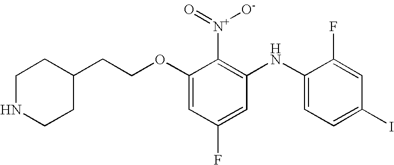

Step 1. Preparation of tert-butyl 4-(2-{5-fluoro-3-[(2-fluoro-4-iodophenyl)amino]-2-nitrophenoxy}ethyl)piperidine-1-carboxylate

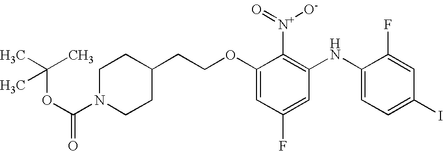

To the solution of tert-butyl 4-(2-hydroxyethyl)piperidine-1-carboxylate (69.8 mg, 0.304 mmol) in dry DMF (3 mL) was added sodium hydride (60%, 20.3 mg, 0.507 mmol), and the mixture was stirred for 10 min, followed by the addition of 3,5-difluoro-N-(2-fluoro-4-iodophenyl)-2-nitroaniline (100 mg, 0.254 mmol) (Example 1). The mixture was stirred for 5 h at rt. LC/MS indicated the reaction was processing, but very slow. The reaction mixture was then heated to 90° C. and stirred at same temperature overnight, cooled to rt, quenched with 5% aq HOAc (20 mL). The mixture was extracted with EtOAc, and the combined organic layers were dried over sodium sulfate. The solvent was evaporated and the residue purified by preparative TLC (DCM/methanol=6:1) to give 70 mg (45.7%) product. ES/MS m/z 625.8 (M+Na⁺); HPLC RT (min) 4.72.

Step 2. Preparation of 5-fluoro-N-(2-fluoro-4-iodophenyl)-2-nitro-3-(2-piperidin-4-ylethoxy)anilin

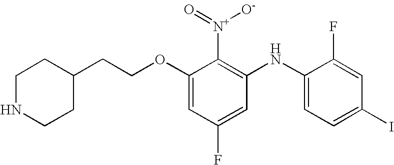

To the solution of tert-butyl 4-(2-{5-fluoro-3-[(2-fluoro-4-iodophenyl)amino]-2-nitro-phenoxy}ethyl)piperidine-1-carboxylate (66.0 mg, 0.109 mmol) in ACETONITRILE (1.5 mL), was added conc. aq HCl (0.15 mL) which was followed by stirring at rt for 1 h. The reaction was quenched with 5% sodium bicarbonate, and the mixture extracted with EtOAc. The organic layer was dried over sodium sulfate and the solvent removed under reduced pressure. The crude product was purified by preparative TLC (DCM/methanol=4:1) to give the product (43.0 mg, 78%). ¹H NMR (400 MHz, CD₃OD), 7.48 (d, 1H), 7.42 (d, 1H), 6.97 (t, 1H), 6.41 (d, 1H), 6.12 (d, 1H), 4.07 (t, 2H), 3.27-3.30 (m, 2H), 2.88 (t, 2H), 1.89-1.92 (m, 3H), 1.79 (m, 1H), 1.71 (m, 2H), 1.34 (m, 2H); ES/MS m/z 504.1 (MH⁺); HPLC RT (min) 4.33.

### Example 1.3

### 2-(3,4-Dihydroxybutoxy)-4-fluoro-6-[(2-fluoro-4-iodophenyl)amino]benzonitrile

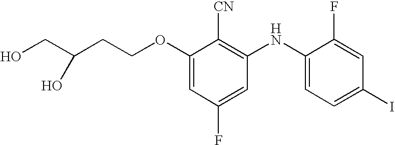

Step 1. Preparation of 2-[2-(2,2-dimethyl-1,3-dioxolan-4-yl)ethoxy]-4-fluoro-6-[(2-fluoro-4-iodophenyl)amino]benzonitrile

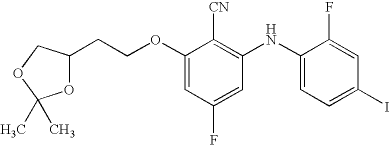

To a solution of 2,4,6-trifluorobenzonitrile (157 mg, 1 mmol) and 2-(2,2-dimethyl-1,3-dioxolan-4-yl)ethanol (146 mg, 1 mmol) in THF (5 mL) was added sodium hydride (60%, 44.0 mg, 1.10 mmol), and the mixture was stirred at rt for 1 h. 2-Fluoro-4-iodoaniline (237 mg, 1 mmol) was added to the above mixture followed by addition of potassium tert-butoxide (135 mg, 1.20 mmol) and stirring at rt for 3 h. The reaction mixture was poured into a mixture of EtOAc (20 mL), water (5 mL), and acetic acid (0.1 mL), and the resulting suspension was stirred for 10 min. The organic layer was separated and dried over sodium sulfate. The solvent was removed under reduced pressure, and the residue purified by prep. TLC (Hex/EtOAc=4/1) to give the product (160 mg, 32%). ES/MS m/z 500.8 (MH⁺); HPLC RT (min) 5.43.

Step 2. Preparation of 2-(3,4-dihydroxybutoxy)-4-fluoro-6-[(2-fluoro-4-iodophenyl)amino]-benzonitrile

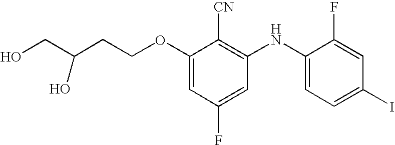

To the solution of 2-[2-(2,2-dimethyl-1,3-dioxolan-4-yl)ethoxy]-4-fluoro-6-[(2-fluoro-4-iodophenyl)amino]benzonitrile (40.0 mg, 0.08 mmol) in acetonitrile (1.5 mL), was added conc. HCl (0.15 mL), the mixture was stirred at rt for 1 h. The reaction was quenched with 5% sodium bicarbonate. The mixture was extracted with EtOAc, and the combined organic layers were dried over sodium sulfate. The solvent was removed under reduced pressure, and the crude product purified by preparative TLC (DCM/methanol=5:1) to give 33.0 mg (90%) of the product. ¹H NMR (400 MHz, CDCl₃), 7.43-7.50 (m, 2H), 7.04 (t, 1H), 6.32 (s, 1H), 6.22 (d, 1H), 6.15 (d, 1H), 4.15-4.21 (m, 2H), 4.04 (b, 1H), 3.71 (b, 1H), 3.55 (b, 1H), 2.82 (b, 2H), 1.92-2.02 (m, 2H); ES/MS m/z 499.96 (M−H⁺); HPLC RT (min) 3.24

### Example 1.4

### 2-[2-(2,2-Dimethyl-1,3-dioxolan-4-yl)ethoxy]-4-fluoro-6-[(2-fluoro-4-iodophenyl)-amino]benzamide

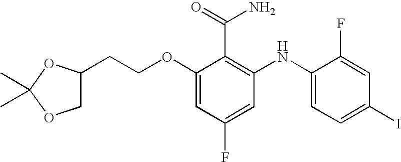

To the solution of 2-[2-(2,2-dimethyl-1,3-dioxolan-4-yl)ethoxy]-4-fluoro-6-[(2-fluoro-4-iodophenyl)amino]benzonitrile (90%, 5.00 g, 9 mmol) in DMSO (20 mL) was added sodium hydroxide (1.37 g, 9.89 mmol solution in water (3.5 mL). The resulting solution was stirred at 63° C. while hydrogen peroxide was added in portions (4×5 mL) within 20 min. The solution was stirred at 63° C. for another 30 min after addition of hydrogen peroxide, cooled to rt, and the mixture was poured into ice-water (50 mL). The pH of the mixture was adjusted to 7 by addition of acetic acid. The resulting precipitate was collected by filtration, washed with water, and dried in vacuo. The crude product was purified by silica gel flash chromatography (120 g column, EtOAC/Hex from 5% to 30%) to give the product (1.55 g, 33%). ¹H NMR (400 MHz, CDCl₃), 8.08 (b, 1H), 7.46 (dd, 1H), 7.40 (d, 1H), 7.08 (t, 1H), 6.34 (d, 1H), 6.10 (dd, 1H), 5.75 (b, 1H), 4.02-4.27 (m, 4H), 3.59 (t, 1H), 2.02-2.15 (m, 2H), 1.39 (s, 3H), 1.31 (s, 3H); ES/MS m/z 519.1 (MH⁺); HPLC RT (min) 4.10.

### Example 1.5

### 2-(3,4-Dihydroxybutoxy)-4-fluoro-6-[(2-fluoro-4-iodophenyl)amino]benzamide

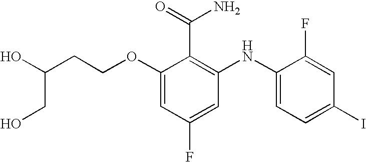

To the solution of 2-[2-(2,2-dimethyl-1,3-dioxolan-4-yl)ethoxy]-4-fluoro-6-[(2-fluoro-4-iodophenyl)amino]benzamide (3.10 g, 5.98 mmol) (Example 4) in THF (15 mL), was added conc. aq. HCl (4 mL), the mixture was stirred at rt for 30 min. The reaction was quenched with 5% sodium bicarbonate (aqueous solution). The solvent was reduced to 5 mL, and the produced crystals were collected by filtration to give the product (2.35 g, 80%). ¹H NMR (400 MHz, CD₃OD), 10.3 (s, 1H), 7.80 (d, 1H), 7.66 (d, 1H), 7.48 (d, 1H), 7.21 (t, 1H), 6.49 (d, 1H), 6.42 (d, 1H), 4.75 (d, 1H), 4.61 (t, 1H), 4.12-4.18 (m, 2H), 3.60-3.64 (m, 1H), 3.24-3.36 (m, 3H), 1.95-1.98 (m, 1H), 1.65-1.70 (m, 1H); ES/MS m/z 479.0 (MH⁺); HPLC RT (min) 4.78.

Using appropriate starting materials and the experimental procedures described above, compounds in Table 1 were prepared. It will be understood by those skilled in the art that some minor modifications to the described procedures may have been made, but such modifications do not significantly affect the results of the preparation.

TABLE 1

| | | | | Prep. |
|---|---|---|---|---|
| | | | | (Ref. |
| Example | | LC-MS | LC-MS RT | Example |
| No. | Structure | m/z (MH⁺) | [min] | No.) |
| 1.1 | 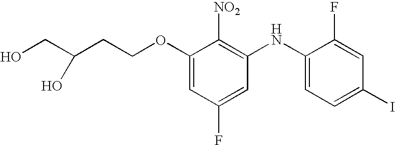 | 480.9 | 4.93 | 1 |
| 1.2 | 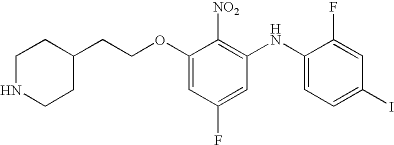 | 504.1 | 4.33 | 1 |
| 1.3 | 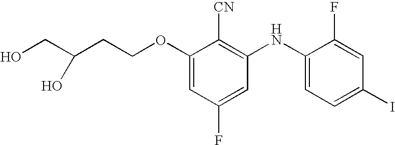 | 461.0 | 4.82 | 2 |
| 1.4 | 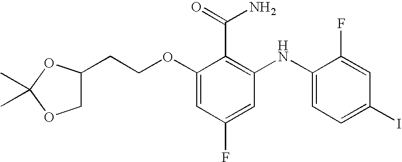 | 519.1 | 4.10 | 3 |
| 1.5 | 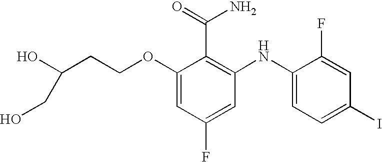 | 479.0 | 4.78 | 3 |
| 1.6 | 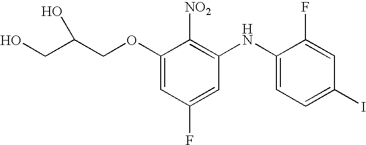 | 466.50 | 4.88 | 2 |
| 1.7 | 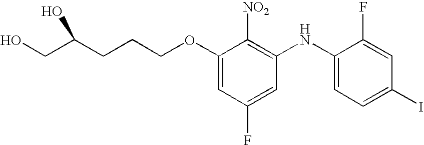 | 494.9 | 4.97 | 2 |
| 1.8 | 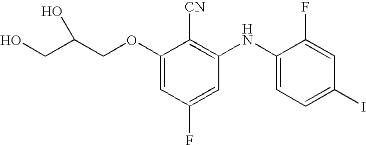 | 447.0 | 3.27 | 2 |
| 1.9 | 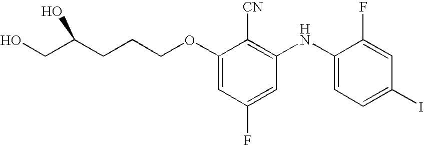 | 475.0 | 4.87 | 2 |
| 1.10 | 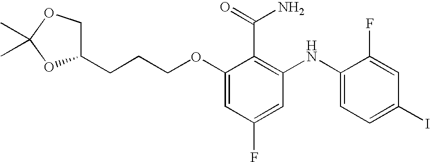 | 533.2 | 4.21 | 3 |
| 1.11 | 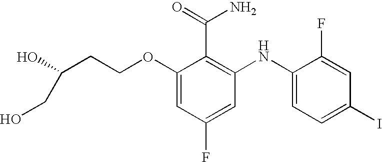 | 501.0(M + Na)⁺ | 3.29 | 3 |
| 1.12 | 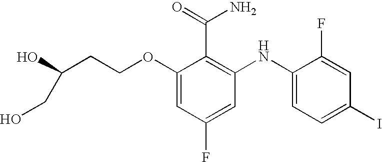 | 501.0(M + Na)⁺ | 3.31 | 3 |
| 1.13 | 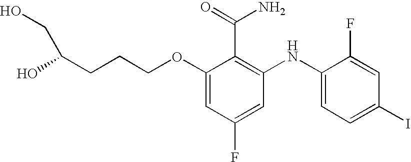 | 493.0 | 4.84 | 3 |

### General Procedures

In the subsequent paragraphs detailed general procedures for the synthesis of key intermediates and compounds of the present invention are described.

### General Procedure 1a (GP 1a): Introduction of C6 Side Chain (Conditions A)

The respective 6 fluoro benzene was dissolved in THF and an alcohol R^{6a}OH (1.01 eq.) [Formula (III) where X=O], a thiol R^{6a}SH (1.01 eq.) [Formula (III) where X═S], or an amine R^{6a}NH₂ (1.01 eq.) [Formula (III) where X═NH] was added. The mixture was treated with sodium hydride (2.01 eq.) and stirred at rt for 48 h. The reaction mixture was poured onto ice water and extracted three times with ethyl acetate. The combined organic layers were washed one time with brine, dried over sodium sulfate, filtered off and concentrated to afford the crude product which was optionally further purified by flash column chromatography, trituration or preparative HPLC purification.

### General Procedure 1b (GP 1b): Introduction of C6 Side Chain (Conditions B)

The respective 6 fluoro benzene was dissolved in DMF, caesium carbonate (1-4 eq.) was added and the mixture allowed to stir at RT for 30 Min. Then molecular sieves were added, followed by the addition of an alcohol R^{6a}OH (1.2 eq.) [Formula (III) where X=O], a thiol R^{6a}SH (1.2 eq.) [Formula (III) where X═S], or an amine R^{6a}NH₂ (1.2 eq.) [Formula (III) where X═NH] in DMF. The mixture was stirred in a sealed pressure tube for 2-48 h. Ethyl methyl ketone was added and the mixture was washed with half concentrated brine twice. The combined organic layers were concentrated to afford the crude product which was optionally further purified by flash column chromatography, trituration or preparative HPLC purification.

### General Procedure 1c (GP 1c): Introduction of C6 Side Chain (Conditions C)

The respective 6 fluoro benzene was dissolved in THF, KtOBu (1-2 eq.) was added and the mixture allowed to stir at RT for 30 Min. Then a solution of an alcohol R^{6a}OH (1.2 eq.) [Formula (III) where X=O], a thiol R^{6a}SH (1.2 eq.) [Formula (III) where X═S], or an amine R^{6a}NH₂ (1.2 eq.) [Formula (III) where X═NH] in DMF was added. The mixture was stirred at 70° C. for 1-24 h. The mixture was partitioned between half concentrated brine and ethyl acetate and extracted twice with ethyl acetate. The combined organic layers were dried over sodium sulphate, filtered off and concentrated to afford the crude product which was optionally further purified by flash column chromatography, trituration or preparative HPLC purification.

### General Procedure 2 (GP 2): Introduction of C2 Side Chain

1 eq of the 2-fluorophenyl substrate and 1.5 eq. of the 2,4-disubstituted benzenamine was dissolved in dry THF. Upon cooling to −60° C., 2-3 eq. of potassium tert-butoxide were added and the mixture was stirred for 30 min at this temperature. The mixture was allowed to warm to rt and was stirred until complete consumption of the starting material. The mixture was then concentrated to afford the crude product which was optionally further purified by flash column chromatography, trituration or preparative HPLC purification.

### General Procedure 3 (GP 3): Hydrolysis of the Benzonitrile

The benzonitrile was dissolved in DMSO and 3 M aq. sodium hydroxide solution (1,1 eq) was added. The mixture was heated to 63° C. and hydrogen peroxide solution (aq., 30%, 10-80 eq.) was added slowly. The mixture was stirred for another 2 h at 65° C. (bath temp.) and then at rt until TLC or LCMS analysis showed no more turnover. The reaction mixture was poured onto ice water and extracted three times with ethyl acetate. The organic layer was washed one time with brine, dried over sodium sulfate, filtered off and concentrated to afford the crude product which was optionally further purified by flash column chromatography, trituration or preparative HPLC purification.

### General Procedure 4a (GP 4a): Clevage of Protecting Groups (BOC Group).

1 eq. of the Boc-protected substrate was suspended in dichloromethane and treated with excess TFA (5-20 eq.). The mixture was subsequently stirred at rt until complete consumption of the starting material. The reaction mixture was concentrated, redissolved in dichloromethane and sodium hydroxide solution (1M, aq.) was added. After phase separation the organic phase was concentrated to afford the crude product which was optionally further purified by flash column chromatography, trituration or preparative HPLC purification.

### General Procedure 4b (GP 4b): Clevage of Protecting Groups (Acetonides).

1 eq. of the acetonide-protected substrate was dissolved in THF. Then hydrochloric acid (aq.; 37%) was added, and the solution was stirred at rt until complete consumption of the starting material. The mixture was concentrated to afford the crude product which was optionally further purified by flash column chromatography, trituration or preparative HPLC purification.

### General Procedure 5 (GP 5): Preparation of Sulfamides

The respective amine was dissolved in DCM and treated subsequently with N-Ethyl-N,N-diisopropyl amin (1.2 eq.). The solution was cooled to 0° C. for 60 min, treated with the respective sulfamoyl chloride (1.1 eq.) and stirred for 30 min at 0° C. and then at RT until TLC or LCMS analysis showed final turnover. Optionally additional equivalents of base and reagent were added to achieve complete turnover. The formed suspension was filtered off, the precipitate was washed with DCM and then dried to afford the pure target compound, which was optionally further purified by flash column chromatography, trituration or preparative HPLC purification.

### General Procedure 6 (GP 6): Preparation of Sulfonamides

The respective amine was dissolved in dichloromethane and 1.2 eq. of pyridine were added. Optionally dichloromethane was replaced by DMF and pyridine was replaced by N-Ethyl-N,N-diisopropyl amin. The mixture was cooled to 3° C. for 10 min before 1.05 eq. of the respective sulfonyl chloride were added. The mixture was stirred at rt until TLC or LCMS analysis showed final turnover. Optionally additional equivalents of base and reagent were added to achieve complete turnover. The reaction mixture was diluted with DCM, washed with aqueous half concentrated sodium bicarbonate solution and the aqueous layer extracted twice with DCM. The combined organic layers were dried and concentrated to afford the crude product, which was optionally further purified by flash column chromatography, trituration or preparative HPLC purification.

### General Procedure 7 (GP 7): Preparation of Ureas

The respective amine (1 eq.) was dissolved in DMF and treated subsequently with 1.2 eq. triethylamine and 1.2 eq. of the respective carbamoyl chloride. The reaction mixture was stirred at rt until TLC or LCMS analysis showed final turnover. Optionally, additional equivalents of amine and carbamoyl chloride were added to achieve complete turnover. The reaction mixture was subsequently quenched with water, extracted with DCM, the combined organic layers were dried and concentrated in vacuo. Flash column chromatography or trituration or preparative HPLC purification provided the target compound.

### General Procedure 8 (GP 8): Preparation of Amides

The respective amine (1 eq.) was dissolved in DCM and treated with N-Ethyl-N,N-diisopropyl amin (1.2 eq.). Upon cooling to 0° C., the respective carboxylic acid chloride (1.01 eq.) was added and the mixture was stirred at rt until TLC or LCMS analysis showed final turnover. The suspension was filtered off, the precipitate washed with DCM, dried and concentrated to afford the crude target compound, which was optionally further purified by flash column chromatography, trituration or preparative HPLC purification.

### General Procedure 9 (GP 9): BOC Protection of the Diphenyl Amine

The diphenyl amine derivative (1 eq.) was dissolved in THF under Argon and DMAP (0.28 eq.) as well as Di-tert-butyldicarbonate (1.56 eq.) were added. The mixture was stirred at rt until TLC or LCMS analysis showed final turnover. The mixture was concentrated to afford the crude target compound, which was optionally further purified by flash column chromatography, trituration or preparative HPLC purification.

### General Procedure 10 (GP 10): Deprotection of the Diphenyl Amine

The respective BOC protected diphenyl amine (1 eq.) was dissolved in DCM, then TFA (20 eq.) was added. The mixture was stirred at RT rt until TLC or LCMS analysis showed final turnover and then concentrated. The residue was partitioned between ethyl methyl ketone and 1 M aq. sodium hydroxide solution. Then the aqueous layer was extracted twice with ethyl methyl ketone. The combined organic layers were washed with half concentrated brine, dried via silicone fitter and concentrated to afford the crude product, which was optionally further purified by flash column chromatography, trituration or preparative HPLC purification.

### General Procedure 11a (GP 11a): Sonogashira Coupling (Conditions A)

The respective iodo-aniline intermediate (1 eq.), bis[(1,2,4,5-eta)-1,5-diphenyl-1,4-pentadien-3-one]-palladium (0.004 eq.), copper(I) iodide (0.004 eq.) and triphenyl-phosphine (0.2 eq.) were weighed into a pressure tube and triethyl amine was added. Upon flushing three times with N₂, trimethylsilyl acetylene (6 eq.) was added, the pressure tube was seated and the resulting suspension was stirred vigorously at 60° C. for 3 h. The mixture was concentrated, redissolved in hexane/ethyl acetate 1:1 and filtered over a NH₂-column (hexane/ethyl acetate 50:50 to 0:100 to pure methanol). The filtrate was concentrated to afford the silylated ethynyl compound.

### General Procedure 11b (GP 11b): Sonogashira Coupling (Conditions B)

The respective iodo-aniline intermediate (1 eq.) was dissolved in THF, together with the respective alkyne (1.5 eq.), followed by dichlorobis(triphenylphosphine)palladium (II) (Pd(PPh₃)₂Cl₂) (0.5 eq.) and a 1M solution of tetra-N-butylammonium fluoride in THF (5 eq.). The mixture was then allowed to react for 40 min at 110° C. in a microwave oven (600 W; max. 6 bar). The crude reaction mixture was directly submitted to preparative HPLC to yield the pure target compound.

### General Procedure 12 (GP 12): Desilylation of Trimethylsilyl Alkynes

To a solution of the respective (trimethylsilyl)alkyne in THF (approx. 10 mL per g alkyne) is added a 1 M solution of tetra-N-butylammonium fluoride in THF (1 eq.), and the resulting mixture is stirred at room temperature until the reaction is completed (typically after approx. 3 h). The product is isolated by dilution with water, extracted with e.g. ethyl acetate and purified by column chromatography (if required).

### General Procedure 13 (GP 13): Bishydroxylation of the C6 Side Chain

The alkene was dissolved in acetone (60-70 ml per mmol alkene) and H₂O (10-11 ml per mmol alkene), N-methyl-morpholino-N-oxide (1.01-1.9 eq.) was added and the mixture cooled to +3° C. An osmiumtetroxide solution (2.5 weight % in t-BuOH, 0.037-0.1 eq.) was added and the mixture was stirred for 40 min in an ice bath and then at rt until TLC or LCMS analysis showed final turnover. Optionally additional equivalents of N-methyl-morpholino-N-oxide and osmiumtetroxide were added to achieve complete turnover. The reaction mixture was concentrated, water and ethyl acetate were added and the organic layer was extracted three times with ethyl acetate. The combined organic layers were washed one time with brine, dried over sodium sulfate, filtered off, concentrated and optionally further purified by flash column chromato-graphy, trituration or preparative HPLC purification.

### General Procedure 14 (GP 14): Methansulfonate (Mesylate) Formation

The respective alcohol (1 eq.) was dissolved in NMP, treated with methansulfonyl chloride (1.1 eq.) and collidine (10 eq.) at 0° C. and kept at this temperature until TLC or LCMS analysis showed final turnover. Preparative HPLC purification of the crude reaction mixture provided the target compound. Alternatively, the crude material was used without further purification in the subsequent substitution reaction.

### General Procedure 15 (GP 15): Methansulfonate (Mesylate) Substitution

1 eq. of the mesylate (as prepared by GP 14) was dissolved in DMF (2 mL per 100 mg mesylate), treated with 20 eq. of the respective nucleophile, e.g. an amine, and stirred at rt until TLC or LCMS analysis indicated final turnover. Preparative HPLC purification of the crude reaction mixture provided the target compound.

### Exemplary HPLC Conditions: (“HPLC Conditions A”)

Equipment: Analytical Waters HPLC system Acquity with Waters ZQ 2000 single quad MS detector.

### Column: Aquity BEH C18 2.1×50 1.7 μm.

Conditions: temperature 60° C.; detection wavelength 214 nm; flow rate 0.8 ml/min;
eluents A: 0.1% formic acid in water, B: 0.1% formic acid in ACN; gradient in each case
based on B: 1% to 99% (1.6′) to 99% (0.4′) to 1% (0.1′)

### Exemplary HPLC Conditions: (“HPLC Conditions B”)

Equipment: Analytical Waters HPLC system Acquity with Waters SQD single quad MS detector.

### Column: Aquity BEH C18 2.1×50 1.7 μm.

Conditions: temperature 60° C.; detection wavelength 254 nm; flow rate 0.8 ml/min;
eluents A: 0.1% formic acid in water, B: ACN; gradient in each case based on B: 1% to 99% (1.6°) to 99% (0.4°) to 1% (0.1°)

### Intermediate 1.1

### Preparation of 2-[2-((R)-2,2-Dimethyl-[1,3]dioxolan-4-yl)-ethoxy]-4,6-difluoro-benzonitrile

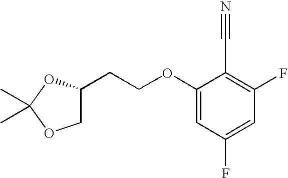

In analogy to GP1a, 5 g of 2,4,6-trifluorobenzonitrile (31.83 mmol, 1 eq; commercially available) and 4.45 ml of 2-((R)-2,2-Dimethyl-[1,3]dioxolan-4-yl)-ethanol (31.83 mmol, 1 eq; commercially available) were dissolved in 150 ml of THF, treated with 2.78 g sodium hydride (62.66 mmol; 2 eq.) and stirred at rt for 2 h. The reaction mixture was poured onto 50 ml of water and extracted three times with 100 ml of ethyl acetate each. The organic layer was washed twice with brine, dried over sodium sulfate, filtered off to afford 5.21 g (57.79% yield, 18.39 mmol) of the desired product.

¹H-NMR (d₆-DMSO; 300 MHz): 6.52-6.57 (m, 2H); 4.30-4.36 (m, 1H); 4.10-4.23 (m, 3H); 3.67 (dd, 1H); 2.11-2.20 (m, 1H); 2.00-2.08 (m, 1H); 1.42 (s, 3H); 1.35 (s, 3H).

MS (ESI): [M+H]⁺=284.

### Intermediate 2.1

### Preparation of N′-[3-(2-cyano-3,5-difluorophenoxy)phenyl]-N,N-dimethyl-sulfamide

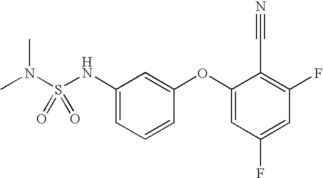

In analogy to GP1a, 430 mg of 2,4,6-trifluorobenzonitrile (2.74 mmol, 1 eq; commercially available) and 596 mg of N′-(3-hydroxyphenyl)-N,N-dimethyl-sulfamide (2.76 mmol, 1.01 eq; commercially available) were dissolved in 25 ml of THF, treated with 240 mg sodium hydride (5.51 mmol, 2.01 eq) and stirred at rt for 48 h. The reaction mixture was poured onto 100 ml of ice water and extracted three times with 70 ml of ethyl acetate each. The organic layer was washed one time with brine, dried over sodium sulfate, filtered off and concentrated to afford 1.06 g of crude product. The concentrate was purified by FlashMaster column chromatography (hexane/ethyl acetate 0-20%) to afford 810 mg (84% yield, 2.29 mmol) of the desired product.

¹H-NMR (d₆-DMSO; 300 MHz): 10.19 (s, 1H); 7.45 (dd, 1H); 7.43 (dd, 1H); 7.13 (ddd, 1H); 7.01 (dd, 1H); 6.92 (dd, 1H); 6.74 (ddd, 1H); 2.72 (s, 6H).

MS (ESI): [M+H]⁺=354.

### Intermediate 2.2

### Preparation of [3-(2-Cyano-3,5-difluoro-phenoxy)-phenyl]-acetic acid tert-butyl ester

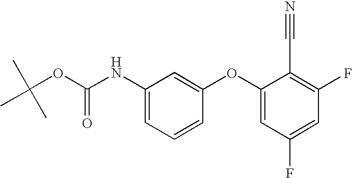

In analogy to GP 1, 3.7 g of 2,4,6-trifluorobenzonitrile (23.6 mmol, 1 eq; commercially available) and 5 g of [3-(2-cyano-3,5-difluoro-phenoxy)-phenyl]-carbamic acid tert-butyl ester (23.9 mmol, 1.01 eq; commercially available) were dissolved in 63 ml of THF, cooled to 0° C. and treated with 2.08 g sodium hydride (47.56 mmol, 2.02 eq.) and stirred at rt for 17 h. The reaction mixture was poured onto 40 ml of ice water and extracted three times with 100 ml of ethyl acetate each. The organic layer was washed one time with brine, dried over sodium sulfate, filtered off and concentrated to afford 9.6 g of crude product. The concentrate was purified by flash chromatography (using hexane/ethyl acetate 99/1-50/50) to afford 5.72 g (70% yield, 16.5 mmol) of the desired product.

¹H-NMR (d₆-DMSO; 300 MHz): 9.57 (s, 1H); 7.39-7.28 (m, 4H); 6.80 (ddd, 1H); 6.62 (ddd, 1H); 1.43 (s, 9H). MS (ESI): [M+H]⁺=347

The following intermediates 2.3 to 2.18 were prepared in analogy to the afore mentioned intermediate compounds by applying general procedure 1a.

| Inter- | | | |
|---|---|---|---|
| mediate | Structure | Name | Analytical data |
| 2.3 | 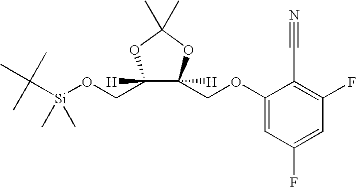 | 2-[(4S,5S)-5-(tert-Butyl-dimethyl-silanyloxymethyl)-2,2-dimethyl-[1,3]dioxolan-4-ylmethoxy]-4,6-difluoro-benzonitrile | MS (ESI):[M + H]⁺ = 414 |
| 2.4 | 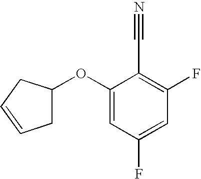 | 2-(Cyclopent-3-enyloxy)-4,6-difluoro-benzonitrile | MS (ESI):[M + H]⁺ = 222 |
| 2.5 | 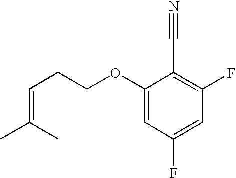 | 2,4-Difluoro-6-(4-methyl-pent-3-enyloxy)-benzonitrile | MS (ESI):[M + H]⁺ = 238 |
| 2.6 | 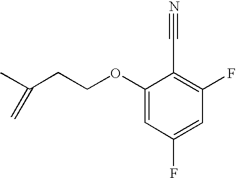 | 2,4-Difluoro-6-(3-methyl-but-3-enyloxy)-benzonitrile | MS (ESI):[M + H]⁺ = 224 |
| 2.7 | 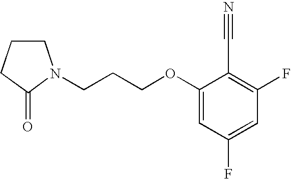 | 2,4-Difloro-6-[3-(2-oxo-pyrrolidin-1-yl)-propoxy]-benzonitrile | MS (ESI):[M + H]⁺ = 281 |
| 2.8 | 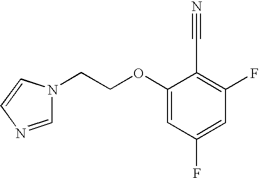 | 2,4-Difluoro-6-(2-imidazol-1-yl-ethoxy)-benzonitrile | MS (ESI):[M + H]⁺ = 250 |
| 2.9 | 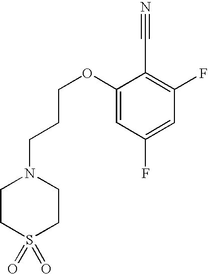 | 2-[3-(1,1-Dioxo-1λ⁶-thiomorpholin-4-yl)-propoxy]-4,6-difluoro-benzonitrile | MS (ESI):[M + H]⁺ = 331 |
| 2.10 | 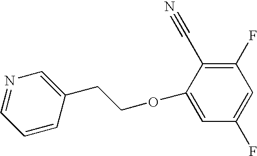 | 2,4-Difluoro-6-(2-pyridin-3-yl-ethoxy)-benzonitrile | MS (ESI):[M + H]⁺ = 261 |
| 2.11 | 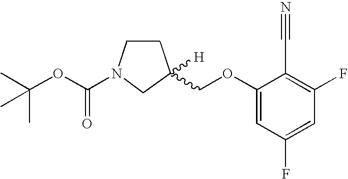 | 3-(2-Cyano-3,5-difluoro-phenoxymethyl)-pyrrolidine-1-carboxylic acid tert-butyl ester | MS (ESI):[M + H]⁺ = 338 |
| 2.12 | 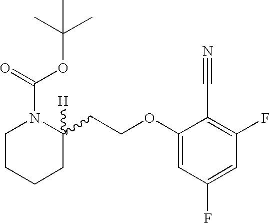 | 2-[2-(2-Cyano-3,5-difluoro-phenoxy)-ethyl]-piperidine-1-carboxylic acid tert-butyl ester | MS (ESI):[M + H]⁺ = 367. |
| 2.13 | 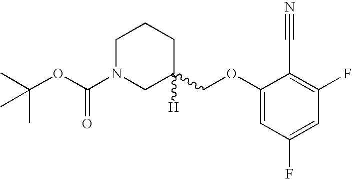 | 3-(2-Cyano-3,5-difluoro-phenoxymethyl)-piperidine-1-carboxylic acid tert-butyl ester | MS (ESI):[M + H]⁺ = 353. |
| 2.14 | 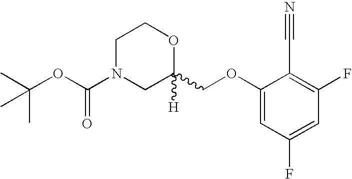 | 2-(2-Cyano-3,5-difluoro-phenoxymethyl)-morpholine-4-carboxylic acid tert-butyl ester | MS (ESI):[M + H]⁺ = 354. |
| 2.15 | 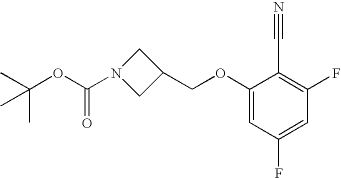 | 3-(2-Cyano-3,5-difluoro-phenoxymethyl)-azetidine-1-carboxylic acid tert-butyl ester | MS (ESI):[M + H]⁺ = 325. |
| 2.16 | 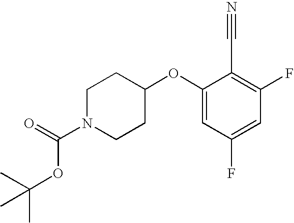 | 4-(2-Cyano-3,5-difluoro-phenoxy)-piperidine-1-carboxylic acid tert-butyl ester | MS (ESI):[M + H]⁺ = 339. |
| 2.17 | 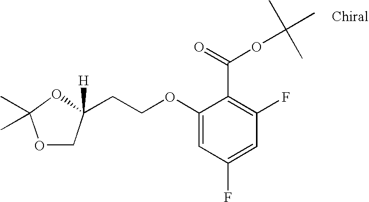 | 2-[2-((R)-2,2-Dimethyl-[1,3]dioxolan-4-yl)-ethoxy]-4,6-difluorobenzoic acid tert-butyl ester | MS (Cl):[M + H]⁺ = 359. |

### Intermediate 3.1 Preparation of 2,4-Difluoro-6-(2-fluoro-4-iodo-phenylamino)-benzonitrile

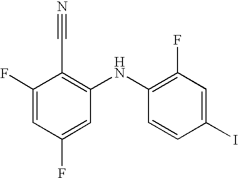

In analogy to GP 2, 1 g of 2,4,6-trifluoro-benzonitrile (6.37 mmol; 1 eq.; commercially available) and 2.26 g 2-fluoro-4-iodo-benzenamine (9.55 mmol, 1.5 eq; commercially available) were dissolved in 100 ml of THF. The mixture was cooled to −65° C.; 2.14 g of potassium tert-butoxide (19.1 mmol, 3 eq; commercially available) were added. The mixture was stirred for 35 min at this temperature and another 21 h at RT. The mixture was stirred into 120 ml of ice water and extracted three times with ethyl acetate (100 ml each). The combined organic layers were washed with brine, dried over sodium sulfate and concentrated to afford 4.137 g of crude product. Purification was achieved by flash chromatography (hexane/ethyl acetate) to afford 646 mg (27.13% yield; 1.73 mmol) of the target compound.

### Intermediate 4.1

### Preparation of 2(2-Cyano-3,5-difluoro-phenyl)-(2-fluoro-4-iodo-phenyl)-carbamic acid tert-butyl ester

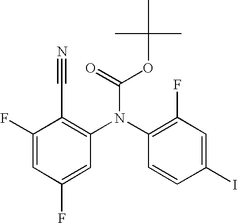

In analogy to GP 9, 205 mg of 2,4-Difluoro-6-(2-fluoro-4-iodo-phenylamino)-benzonitrile (0.55 mmol; 1 eq.) were dissolved in THF under argon and 19 mg DMAP (0.16 mmol; 0.28 eq.) as well as 186 mg of Di-tert-butyldicarbonate (0.85 mmol; 1.56 eq.) were added. The mixture was stirred at RT for 20 h. The mixture was concentrated and purified by flash chromatography (5 g Si-column, using hexane/ethyl acetate 100/0-70/30) to afford 253 mg (97% yield, 0.53 mmol) of the desired product.

The following intermediates 5.1 to 5.14 were prepared in analogy to processes described above and below by nucleophilic displacement of a fluorine by the respective anilines (GP 2) and optionally subsequent nitrile hydrolysis (GP 3).

| Inter- | | | |
|---|---|---|---|
| mediate | Structure | Name | Analytical Data |
| 5.1 | 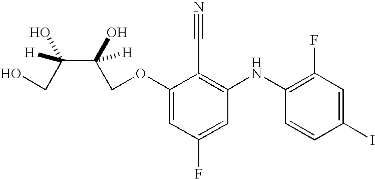 | 4-Fluoro-2-(2-fluoro-4-iodo-phenylamino)-6-((2S,3S)-2,3,4-trihydroxy-butyoxy)-benzonitrile | MS (ESI):[M + H]+ = 477. |
| 5.2 | 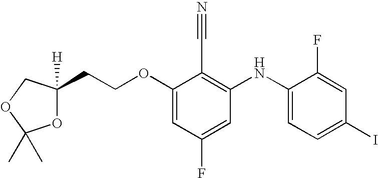 | 2-[2-((R)-2,2-Dimethyl-[1,3]dioxolan-4-yl)-ethoxy]-4-fluoro-6-(2-fluoro-4-iodo-phenylamino)-benzonitrile | 1H-NMR:(CDCl3, 300 MHz)7.46-7.54 (m, 2 H); 7.08 (t,1 H); 6.25-6.30 (m, 2H); 6.18 (dd, 1 H); 4.29-4.28(m, 1 H); 4.00-4.20 (m, 3 H);3.68 (dd, 1 H); 1.94-2.20 (m,2 H); 1.43 (s, 3 H); 1.38 (S, 33H). |
| 5.3 | 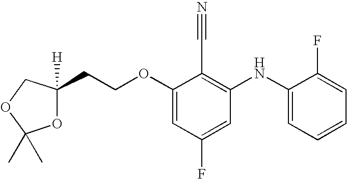 | 2-[2-((R)-2,2-Dimethyl-[1,3]dioxolan-4-yl)-ethoxy]-4-fluoro-6-(2-fluoro-phenylamino)-benzonitrile | MS (ESI):[M + H]+ = 375. |
| 5.4 | 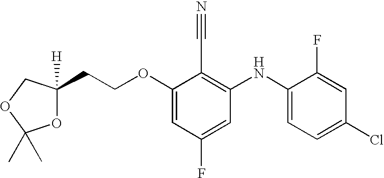 | 2-(4-Chloro-2-fluoro-phenylamino)-6-[2-((R)-2,2-dimethyl-[1,3]dioxolan-4-yl)-ethoxy]-4-fluoro-benzonitrile | MS (ESI):[M + H]+ = 409. |
| 5.5 | 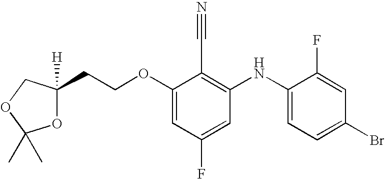 | 2-(4-Bromo-2-fluoro-phenylamino)-6-[2-((R)-2,2-dimethyl-[1,3]dioxolan-4-yl)-ethoxy]-4-fluoro-benzonitrile | MS (ESI):[M + H]+ = 454. |
| 5.6 | 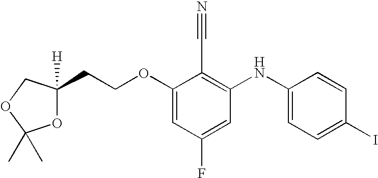 | 2-[2-((R)-2,2-Dimethyl-[1,3]dioxolan-4-yl)-ethoxy]-4-fluoro-6-(4-iodo-phenylamino)-benzonitrile | MS (ESI):[M + H]+ = 483. |
| 5.7 | 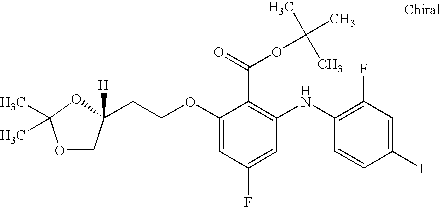 | 2-[2-((R)-2,2-Dimethyl-[1,3]dioxolan-4-yl)-ethoxy]-4-fluoro-6-(2-fluoro-4-iodo-phenylamino)-benzoic acid tert-butyl ester | MS (ESI):[M + H]+ = 576. |
| 5.8 | 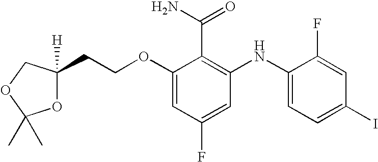 | 2-[2-((R)-2,2-Dimethyl-[1,3]dioxolan-4-yl)-ethoxy]-4-fluoro-6-(2-fluoro-4-iodo-phenylamino)-benzonitrile | 1H-NMR:(CDCl3, 300 MHz)10.86 (s, 1 H); 8.06 (br. s, 1H); 7.46 (dd, 1 H); 7.41 (dd, 1H); 7.10 (t, 1 H); 6.36 (ddd, 1H); 6.10 (dd, 1 H); 5.79 (br. s,1 H); 4.09-4.30 (m, 4 H);3.60 (t, 1 H); 2.02-2.12 (m,2 H); 1.41 (s, 3 H); 1.34 (s, 3H).MS (ESI):[M + H]+ = 519. |
| 5.9 | 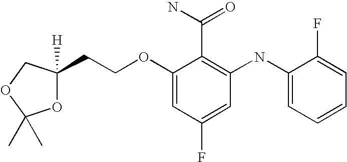 | 2-[2-((R)-2,2-Dimethyl-[1,3]dioxolan-4-yl)-ethoxy]-4-fluoro-6-(2-fluoro-phenylamino)-benzamide | MS (ESI):[M + H]+ = 393. |
| 5.10 | 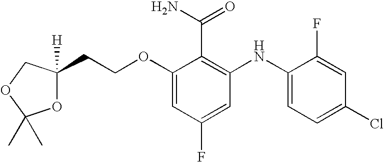 | 2-(4-Chloro-2-fluoro-phenylamino)-6-[2-((R)-2,2-dimethyl-[1,3]dioxolan-4-yl)-ethoxy]-4-fluoro-benzamide | MS (ESI):[M + H]+ = 427. |
| 5.11 | 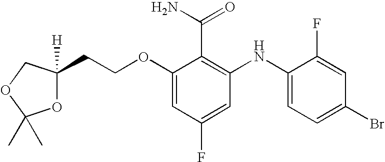 | 2-(4-Bromo-2-fluoro-phenylamino)-6-[2-((R)-2,2-dimethyl-[1,3]dioxolan-4-yl)-ethoxy]-4-fluoro-benzamide | 1H-NMR:(CDCl3, 300 MHz)10.89 (br. s, 1 H); 8.15 (br. s,1 H); 7.24-7.37 (m, 4 H);6.37 (br. d, 1 H); 6.15 (dd, 1H); 5.76 (br. s, 1 H); 4.15-4.35 (m, 4 H); 3.65 (t, 1 H);2.08-2.23 (m, 2 H); 1.46 (s,3 H); 1.41 (s, 3 H).MS (ESI):[M + H]+ = 472. |
| 5.12 | 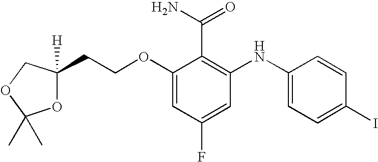 | 2-[2-((R)-2,2-Dimethyl-[1,3]dioxolan-4-yl)-ethoxy]-4-fluoro-6-(4-iodo-phenylamino)-benzamide | 1H-NMR:(CDCl3, 300 MHz)10.80 (br. s, 1 H); 8.08 (br. s,1 H); 7.61 (d, 2 H); 6.97 (d, 2H); 6.51 (dd, 1 H); 6.07 (dd, 1H); 5.66 (br. s, 1 H); 4.08-4.29 (m, 4 H); 3.61 (t, 1 H);2.05-2.13 (m, 2 H); 1.41 (s,3 H); 1.35 (s, 3 H).MS (ESI):[M + H]+ = 501. |
| 5.13 | 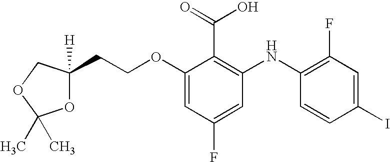 | 2-[2-((R)-2,2-Dimethyl-[1,3]dioxolan-4-yl)-ethoxy]-4-fluoro-6-(2-fluoro-4-iodo-phenylamino)-benzoic acid | MS (ESI):[M + H]+ = 520. |
| 5.14 | 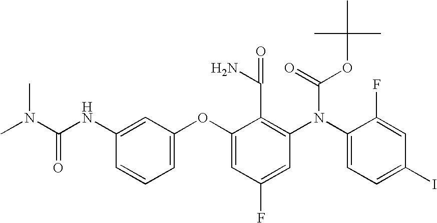 | {2-Carbamoyl-3-[3-(3,3-dimethyl-ureido)-phenoxy]-5-fluoro-phenyl}-(2-fluoro-4-iodo-phenyl)-carbamicacid tert-butyl ester | MS (ESI):[M + H]+ = 651. |

### Intermediate 6.1:

### Preparation of {2-Cyano-3-[3-(3,3-dimethyl-ureido)-phenoxy]-5-fluoro-phenyl}-(2-fluoro-4-iodo-phenyl)-carbamic acid tert-butyl ester

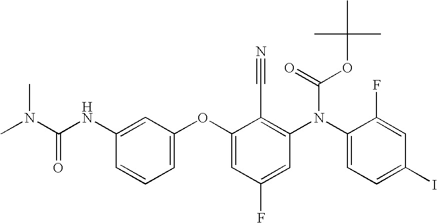

In analogy to GP 1a, 100 mg of {2-Cyano-3-[3-(3,5-difluorophenyl)-(2-fluoro-4-iodo-phenyl)-carbamic acid tert-butyl ester (0.21 mmol, 1 eq.) and 39.14 mg of N′-(3-hydroxyphenyl)-N,N-diphenylsulfamide (0.22 mmol, 1.03 eq; commercially available) were dissolved in 5 ml THF and treated with 24.84 mg sodium hydride (0.57 mmol; 2.7 eq.) and stirred at rt for 27 h. The reaction mixture was poured onto 20 ml of ice water and extracted three times with 30 ml of ethyl acetate each. The organic layer was washed one time with brine, dried over sodium sulfate, filtered off and concentrated to afford 160 mg of crude product. The concentrate was purified by flash chromatography to afford 53 mg (40.2% yield, 0.085 mmol) of the desired product.

MS (ESI) [M+H]⁺=635.

### Example 2.1

### Preparation of N′-[3-[2-cyano-5-fluoro-3-[(2-fluoro-4-iodophenyl)amino]phenoxy]phenyl]-N,N-dimethyl-sulfamide

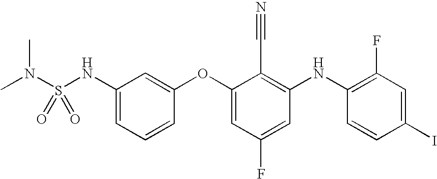

In analogy to GP 2, 410 mg of N′-[3-(2-cyano-3,5-difluorophenoxy)phenyl]-N,N-dimethyl-sulfamide (1.16 mmol, 1 eq) and 413 mg of 2-fluoro-4-iodo-benzenamine (1.74 mmol, 1.5 eq; commercially available) were dissolved in 20 ml of THF. Upon cooling to −60° C., 393 mg of potassium tert-butoxide were added and the mixture stirred for 30 min at this temperature. The mixture was allowed to warm to rt slowly and was stirred for another 22 h at rt. The mixture was then concentrated and purified (FlashMaster column chromatography, hexane/ethyl acetate 0-30%) to afford 354 mg of the desired product.

¹H-NMR (d₆-DMSO; 300 MHz): 10.15 (s, 1H); 8.84 (s, 1H); 7.75° (dd, 1H); 7.58 (ddd, 1H); 7.40 (dd, 1H); 7.15 (dd, 1H); 7.08 (ddd, 1H); 6.96 (dd, 1H); 6.87 (ddd, 1H); 6.28 (ddd, 1H); 6.18 (dd, 1H); 2.72 (s, 6H).

MS (ESI): [M+H]⁺=571

### Example 2.2

### Preparation of {3-[2-Cyano-5-fluoro-3-(2-fluoro-4-iodo-phenylamino)-phenoxy]-phenyl}-carbamic acid tert-butyl ester

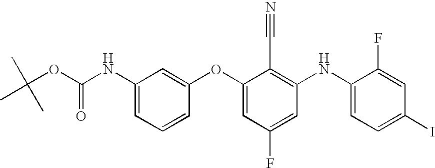

In analogy to GP 2, 500 mg of [3-(2-Cyano-3,5-difluoro-phenoxy)-phenyl]-acetic acid tert-butyl ester (1.44 mmol, 1 eq) and 513 mg of 2-fluoro-4-iodo-benzenamine (2.17 mmol, 1.5 eq; commercially available) were dissolved in 13 ml of THF. Upon cooling to 3° C., 486 mg (4.33 mmol, 3 eq.) of potassium tert-butoxide were added and the mixture stirred for 30 min at this temperature. The mixture was allowed to come to rt slowly and was stirred for another 20 h at rt. After addition of 162 mg (1.44 mmol, 1 eq.) of potassium tert-butoxide the mixture was stirred at rt for another 2 h. The reaction mixture was poured onto 30 ml of ice water and 30 ml ethyl acetate were added. The aqueous phase was extracted three times with 40 ml of ethyl acetate each. The combined organic Layers were washed one time with brine, dried over sodium sulfate, filtered off and concentrated to afford 750 mg of crude product. The concentrate was purified by flash chromatography (hexane/ethyl acetate 99/1-60/40) to afford 406 g (50% yield, 0.72 mmol) of the desired product.

¹H-NMR (d₆-DMSO; 300 MHz): 9.54 (s, 1H); 8.77 (s, 1H); 7.69 (dd, 1H); 7.53 (dbr, 1H); 7.34-7.24 (m, 3H); 7.11 (dd, 1H); 6.75 (ddd, 1H); 6.21 (ddd, 1H); 6.07 (dd, 1 H); 1.43 (s, 9H).

MS (ESI): [M+H]⁺=564

The following example compounds 2.3 and 2.16 were prepared in analogy to and general procedure 2:

| Example | Structure | Name | Analytical data |
|---|---|---|---|
| 2.3 | 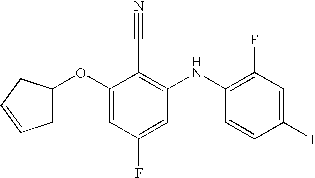 | 2-(Cyclopent-3-enyloxy)-4-fluoro-6-(2-fluoro-4-iodo-phenylamino)-benzonitrile | MS (ESI):[M + H]+ = 439. |
| 2.4 | 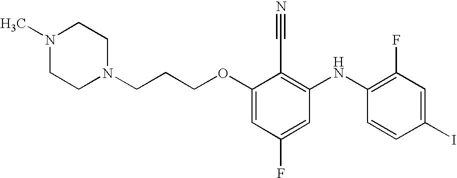 | 4-Fluoro-2-(2-fluoro-4-iodo-phenylamino)-6-[3-(4-methyl-piperazin-1-yl)-propoxy]-benzonitrile | MS (ESI):[M + H]+ = 513. |
| 2.5 | 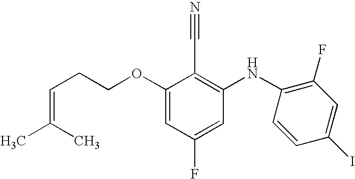 | 4-Fluoro-2-(2-fluoro-4-iodo-phenylamino)-6-(4-methyl-pent-3-enyloxy)-benzontrile | MS (ESI):[M + H]+ = 455. |
| 2.6 | 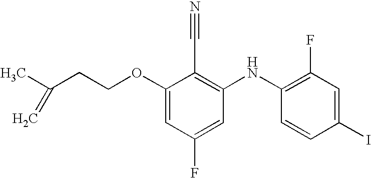 | 4-Fluoro-2-(2-Fluoro-4-iodo-phenyamino)-6-(3-methyl-but-3-enyloxy)-benzontrile | MS (ESI):[M + H]+ = 441. |
| 2.7 | 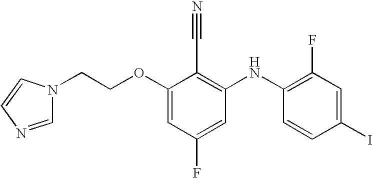 | 4-Fluoro-2-(2-fluoro-4-iodo-phenylamino)-6-(2-imidaozl-1-yl-ethoxy)-benzontrile | MS (ESI):[M + H]⁺ = 466. |
| 2.8 | 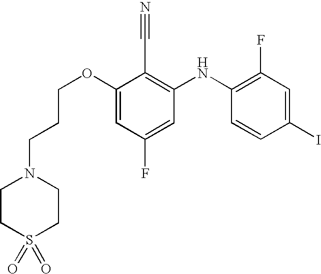 | 2-[3-(1,1-Dioxo-1λ⁶-thiomorpholin-4-yl)-propoxy]-4-fluoro-6-(2-fluoro-4-iodo-phenylamino)-benzonitrile | MS (ESI):[M + H]⁺ = 548. |
| 2.9 | 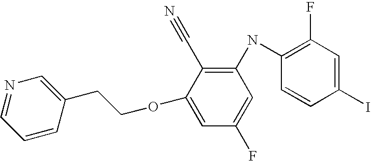 | 4-Fluoro-2-(2-fluoro-4-iodo-phenylamino)-6-(2-pyridin-3-yl-ethoxy)-benzonitrile | MS (ESI):[M + H]⁺ = 478. |
| 2.10 | 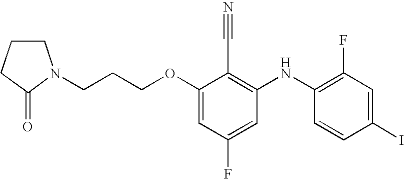 | 4-Fluoro-2-(2-fluoro-4-iodo-phenylamino)-6-[3-(2-oxo-pyrrolidin-1-yl)-propoxy]-benzonitrile | MS (ESI):[M + H]⁺ = 498. |
| 2.11 | 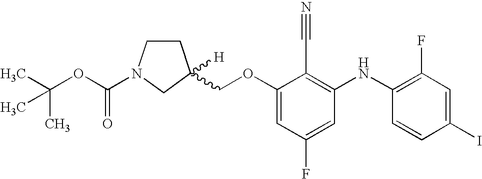 | 3-[2-Cyano-5-fluoro-3-(2-fluoro-4-iodo-phenylamino)-phenoxymethyl]-pyrrolidine-1-carboxylic acid tert-butyl ester | MS (ESI):[M + H]⁺ = 556. |
| 2.12 | 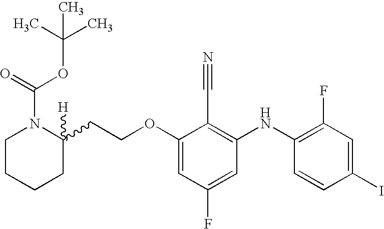 | 2-{2-[2-Cyano-5-fluoro-3-(2-fluoro-4-iodo-phenylamino)-phenoxy]-ethyl}-piperidine-1-carboxylic acid tert-butyl ester | MS (ESI):[M + H]⁺ = 584. |
| 2.13 | 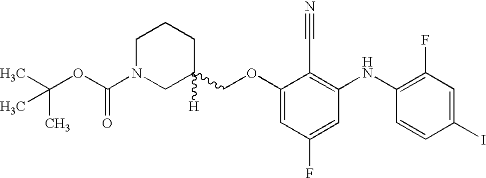 | 3-[2-Cyano-5-fluoro-3-(2-fluoro-4-iodo-phenylamino)-phenoxymethyl]-piperidine-1-carboxylic acid tert-butyl ester | MS (ESI):[M + H]⁺ = 570. |
| 2.14 | 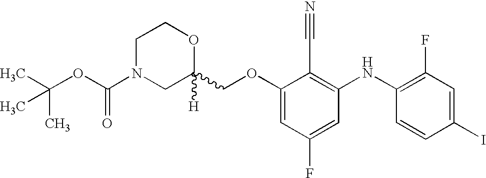 | 2-[2-Cyano-5-fluoro-3-(2-fluoro-4-iodo-phenylamino)-phenoxymethyl]-morpholine-4-carboxylic acid tert-butyl ester | MS (ESI):[M + H]⁺ = 572. |
| 2.15 | 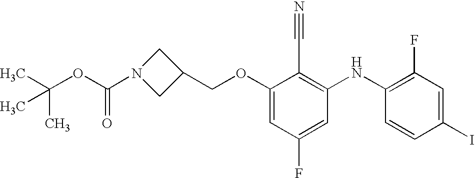 | 3-[2-Cyano-5-fluoro-3-(2-fluoro-4-iodo-phenylamino)-phenoxymethyl]-azetidine-1-carboxylic acid tert-butyl ester | MS (ESI):[M + H]⁺ = 542. |
| 2.16 | 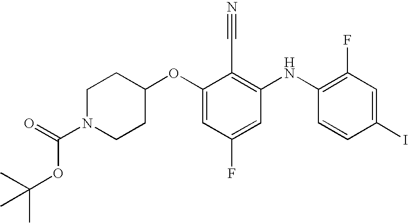 | 4-[2-Cyano-5-fluoro-3-(2-fluoro-4-iodo-phenylamino)-phenoxy]-piperidine-1-carboxylic acidtert-butyl ester | MS (ESI):[M + H]⁺ = 556. |

### Example 3.1

### Preparation of {3-[2-Carbamoyl-5-fluoro-3-(2-fluoro-4-iodo-phenylamino)-phenoxy]-phenyl}-carbamic acid tert-butyl ester

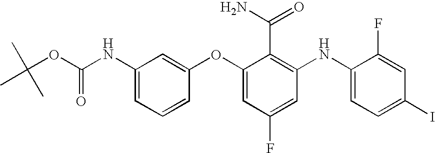

In analogy to GP 3, 386 mg of {3-[2-Cyano-5-fluoro-3-(2-fluoro-4-iodo-phenylamino)-phenoxy]-phenyl}-carbamic acid tert-butyl ester (0.69 mmol, 1 eq) were dissolved in 4.8 ml of DMSO and 0.24 ml of 3 M aq. sodium hydroxide solution (0.72 mmol, 10-80 eq) were added. The mixture was heated to 63° C. and 1.85 ml of hydrogen peroxide solution (aq., 30%) were added over the course of 20 min. The mixture was stirred for another 2 h at 65° C. (bath temp.). The reaction mixture was poured onto 175 ml of ice water. 300 ml of ethyl acetate were added and the phases separated. The aqueous phase was extracted one more time with 150 ml of ethyl acetate. The combined organic layers were washed one time with brine, dried over sodium sulfate, filtered off and concentrated. The concentrate was purified (FlashMaster column chromatography, hexane/ethyl acetate 99/1-60/40) to afford 169 mg (42% yield, 0.29 mmol) of the desired product.

¹H-NMR (d₆-DMSO; 300 MHz): 9.46 (s, 1H); 9.12 (s, 1H); 7.83 (sbr, 2H); 7.66 (dd, 1H); 7.47 (dbr, 1H); 7.30-7.17 (m, 4H); 6.65 (ddd, 1H); 6.54 (dbr, 1H); 6.06 (dd, 1H); 1.42 (s, 9H).

MS (ESI): [M+H]⁺=582

### Example Compound 3.2

### Preparation of 2-[3-[[(dimethylamino)sulfonyl]amino]phenoxy]-4-fluoro-6-[(2-fluoro-4-iodophenyl)amino]-benzamide

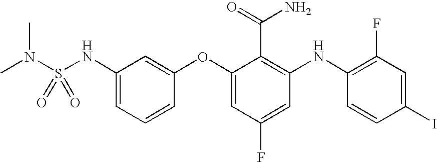

In analogy to GP 3, 210 mg of N′-[3-[2-cyano-5-fluoro-3-[(2-fluoro-4-iodophenyl)amino]phenoxy]phenyl]-N,N-dimethyl-sulfamide (0.37 mmol, 1 eq) were dissolved in 3 ml of DMSO and 0.14 ml of 3 M aq. sodium hydroxide solution (0.41 mmol, 1,1 eq) were added. The mixture was heated to 63° C. and 0.8 ml of hydrogen peroxide solution (aq., 30%) were added during the course of 1.5 h. The mixture was stirred for another 2 h at 65° C. (bath temp.) and for 18 h at rt. The reaction mixture was poured onto 80 ml of ice water and extracted three times with 50 ml of ethyl acetate each. The organic layer was washed one time with brine, dried over sodium sulfate, filtered off and concentrated to afford 402 mg of crude product. The concentrate was purified (FlashMaster column, hexane/ethyl acetate 0-50%) to afford 94 mg (43% yield, 0.16 mmol) of the desired product.

¹H-NMR (d₆-DMSO; 300 MHz): 10.05 (s, 1H); 9.08 (s, 1H); 7.90 (sbr, 1H); 7.87 (sbr, 1H); 7.70 (dd, 1H); 7.52 (ddd, 1H); 7.33 (dd, 1H); 7.25 (dd, 1H); 7.00 (ddd, 1H); 6.94 (dd, 1H); 6.75 (ddd, 1H); 6.61 (ddd, 1H); 6.16 (dd, 1H); 2.71 (s, 6H).

MS (ESI): [M+H]⁺=589

The following example compounds 3.3 to 3.17 were prepared in analogy to example compounds 3.1 and 3.2 by applying GP 3 to the respective nitriles.

| Example | Structure | Name | Analytica data |
|---|---|---|---|
| 3.3 | 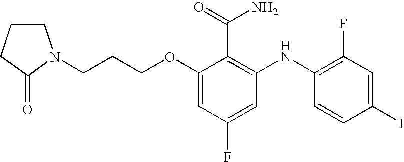 | 4-Fluoro-2-(2-fluoro-4-iodo-phenylamino)-6-[3-(2-oxo-pyrrolidin-1-yl)-propoxy]-benzamide | 1H-NMR:(d6-DMSO, 300 MHz)9.47 (s, 1 H); 7.94 (sbr, 1 H);7.81 (sbr, 1 H); 7.45 (ddd, 1H); 7.19 (dd, 1 H); 6.42 (d, 2H); 3.92 (dd, 2 H); 3.37-3.25(m, 4 H); 2.18 (dd, 2 H); 1.96-1.83 (m, 4 H).MS (ESI):[M + H]+ = 516 |
| 3.4 | 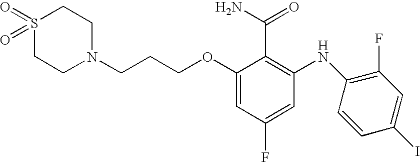 | 2-[3-(1,1-Dioxo-1λ⁶-thiomorpholin-4-yl)-propoxy]-4-fluoro-6-(2-fluoro-4-iodo-phenylamino)-benzamide | 1H-NMR:(d6-DMSO, 400 MHz)9.55 (s, 1 H); 7.76 (s, 1 H);7.65 (s, 1 H); 7.65 (dd, 1 H);7.46 (dd, 1 H); 7.22 (t, 1 H);6.37 (dd, 1 H); 6.33 (br. s, 1H); 3.95 (t, 2 H); 3.03 (m, 4H); 2.84 (m, 4 H); 2.52 (t, 2H); 1.78 (qu, 2 H). |
| 3.5 | 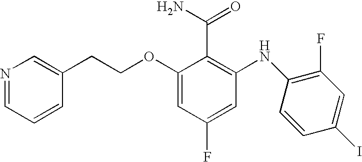 | 4-Fluoro-2-(2-fluoro-4-iodo-phenylamino)-6-(2-pyridin-3-yl-ethoxy)-benzamide | 1H-NMR:(d6-DMSO, 400 MHz)9.71 (s, 1 H); 8.51 (s, 1 H);8.41 (d, 1 H); 7.78 (s, 1 H);7.72 (dt, 1 H); 7.63 (dd, 1 H);7.55 (s, 1 H); 7.46 (d, 1 H);7.30 (dd, 1 H); 7.17 (t, 1 H);6.50 (dd, 1 H); 6.40 (dd, 1 H);3.26 (t, 2 H); 3.09 (t, 2 H). |
| 3.6 | 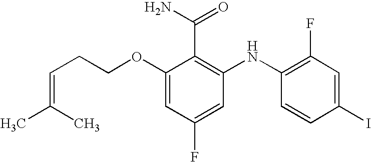 | 4-Fluoro-2-(2-fluoro-4-iodo-phenylamino)-6-(4-methyl-pent-3-enyloxy)-benzamide | MS (ESI):[M + H]⁺ = 473. |
| 3.7 | 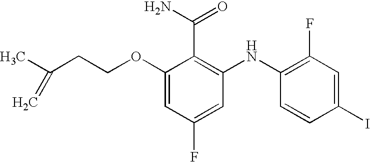 | 4-Fluoro-2-(2-fluoro-4-iodo-phenylamino)-6-(3-methyl-but-3-enyloxy)-benzamide | MS (ESI):[M + H]⁺ = 459. |
| 3.8 | 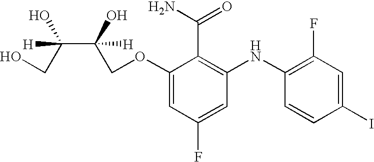 | 4-Fluoro-2-(2-fluoro-4-iodo-phenylamino)-6-((2S,3S)-2,3,4-trihydroxy-butoxy)-benzamide | MS (ESI):[M + H]+ = 495. |
| 3.9 | 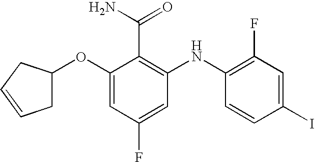 | 2-(Cyclopent-3-enyloxy)-4-fluoro-6-(2-fluoro-4-iodo-phenylamino)-benzamide | MS (ESI):[M + H]+ = 457. |
| 3.10 | 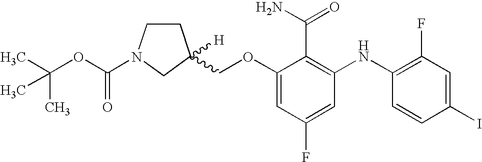 | 3-[2-Carbamoyl-5-fluoro-3-(2-fluoro-4-iodo-phenylamino)-phenoxymethyl]-pyrrolidine-1-carboxylic acid tert-butyl ester | MS (ESI):[M + H]+ = 574. |
| 3.11 | 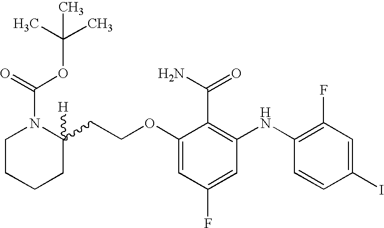 | 2-{2-[2-Carbamoyl-5-fluoro-3-(2-fluoro-4-iodo-phenylamino)-phenoxy]-ethyl}-piperidine-1-carboxylic acid tert-butyl ester | MS (ESI):[M + H]+ = 602. |
| 3.12 | 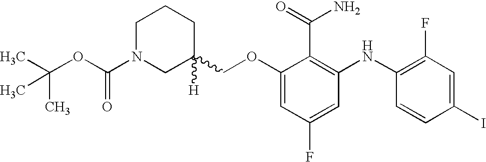 | 3-[2-Carbamoyl-5-fluoro-3-(2-fluoro-4-iodo-phenylamino)-phenoxymethyl]-piperidine-1-carboxylic acid tert-butyl ester | MS (ESI):[M + H]+ = 588. |
| 3.13 | 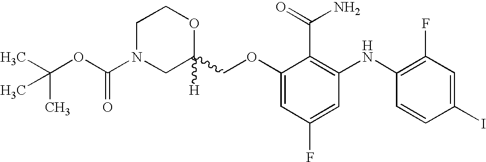 | 2-[2-Carbamoyl-5-fluoro-3-(2-fluoro-4-iodo-phenylamino)-phenoxymethyl]-morpholine-4-carboxylic acid tert-butyl ester | MS (ESI):[M + H]+ = 590. |
| 3.14 | 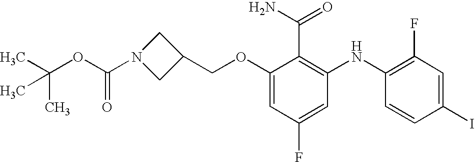 | 3-[2-Carbamoyl-5-fluoro-3-(2-fluoro-4-iodo-phenylamino)-phenoxymethyl]-azetidine-1-carboxylic acid tert-butyl ester | MS (ESI):[M + H]+ = 560. |
| 3.15 | 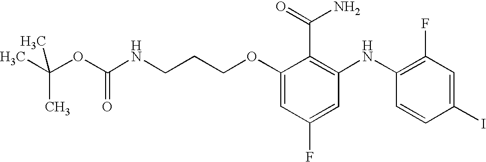 | {3-[2-Carbamoyl-5-fluoro-3-(2-fluoro-4-iodo-phenylamino)-phenoxy]-propyl}-carbamic acid tert-butyl ester | MS (ESI):[M + H]+ = 548. |
| 3.16 | 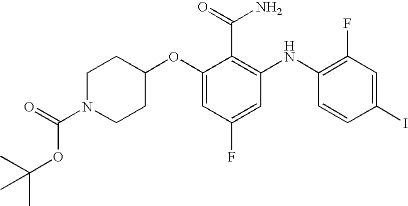 | 4-[2-Carbamoyl-5-fluoro-3-(2-fluoro-4-iodo-phenylamino)-phenoxy]-piperidine-1-carboxylic acidtert-butyl ester | MS (ESI):[M + H]+ = 574. |
| 3.17 | 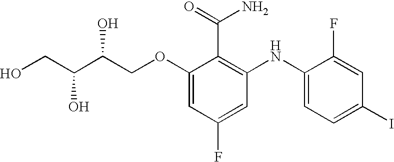 | 4-Fluoro-2-(2-fluoro-4-iodo-phenylamino)-6-((2R,3R)-2,3,4-trifhydroxy-butoxy)-benzamide | MS (ESI):[M + H]+ = 495. |

### Example 4.1

### Preparation of 2-(3-Amino-phenoxy)-4-fluoro-6-(2-fluoro-4-iodo-phenylamino)-benz-amide

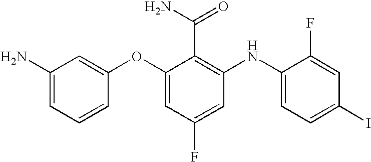

In analogy to GP 4a, 163 mg of {3-[2-Carbamoyl-5-fluoro-3-(2-fluoro-4-iodo-phenylamino)-phenoxy]-phenyl}-carbamic acid tert-butyl ester (0.28 mmol) were suspended in dichloromethane, 0.29 ml of TFA (3.78 mmol, 13 eq.) were added and the mixture was stirred at rt for 4 h. The reaction mixture was concentrated, redissolved in dichloromethane and sodium hydroxide solution (1M, aq.) was added. After phase separation the organic phase was concentrated to afford 129 mg (96%, 0.27 mmol) of the desired product, which required no further purification.

¹H-NMR (d₆-DMSO; 300 MHz): 9.23 (s, 1H); 7.84 (sbr, 1H); 7.77 (sbr, 1H); 7.66 (dd, 1 H); 7.47 (dbr, 1H); 7.21 (dd, 1H); 7.04 (dd, 1H); 6.53 (dbr, 1H); 6.42 (dbr, 1H); 6.31-6.26 (m, 2H); 6.07 (dd, 1H).

MS (ESI): [M+H]⁺=482

The following example compounds 4.2 to 4.9 were prepared in analogy to example compound 4.1 by applying GP 4a (or other standard deprotection conditions as known to the person skilled in the art) to the respective protected substrate, which have been prepared as described above.

| Example | Structure | Name | Analytical data |
|---|---|---|---|
| 4.2 | 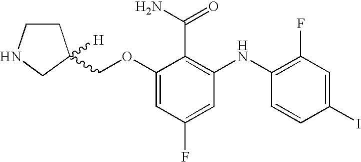 | 4-Fluoro-2-(2-fluoro-4-iodo-phenylamino)-6-(pyrrolidin-3-ylmethoxy)-benzamide | MS (ESI):[M − H]− = 474. |
| 4.3 | 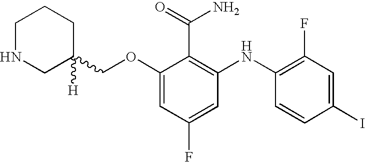 | 4-Fluoro-2-(2-fluoro-4-iodo-phenylamino)-6-(piperidin-3-ylmethoxy)-benzamide | MS (ESI):[M − H]− = 488. |
| 4.4 | 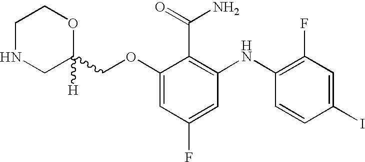 | 4-Fluoro-2-(2-fluoro-4-iodo-phenylamino)-6-(morpholin-2-ylmethoxy)-benzamide | MS (ESI):[M − H]− = 490. |
| 4.5 | 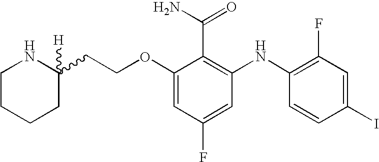 | 4-Fluoro-2-(2-fluoro-4-iodo-phenylamino)-6-(2-piperidin-2-yl-ethoxy)-benzamide | MS (EI):[M − H]− = 502. |
| 4.6 | 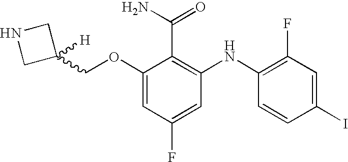 | 2-(Azeitidn-3-ylmethoxy)-4-fluoro-6-(2-fluoro-4-iodo-phenylamino)-benzamide | MS (ESI):[M − H]− = 460. |
| 4.7 | 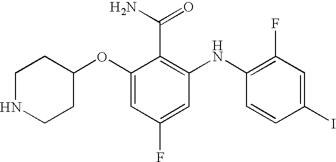 | 4-Fluoro-2-(2-fluoro-4-iodo-phenylamino)-6-(piperidin-4-yloxy)-benzamide | 1H-NMR:(d6-DMSO, 300 MHz)9.74 (s, 1 H); 7.80 (s, 1 H);7.63 (dd, 1 H); 7.59 (s, 1 H);7.45 (dd, 1 H); 7.18 (t, 1 H);6.54 (dd, 1 H); 6.38 (dd, 1 H);4.51 (m, 1 H); 2.82-2.90 (m,2 H); 2.45-2.57 (m, 4 H);1.83-1.92 (m, 2 H).MS (ESI):[M + H]+ = 474. |
| 4.8 | 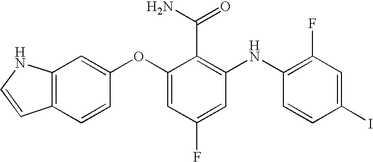 | 4-Fluoro-2-(2-fluoro-4-iodo-phenylamino)-6-(1H-indol-6-yloxy)-benzamide | MS (ESI):[M − H]− = 506. |
| 4.9 | 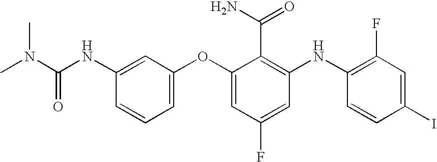 | 2-[3-(3,3-Dimethyl-ureido)-phenoxy]-4-fluoro-6-(2-fluoro-4-iodo-phenylamino)-benzamide | MS (ESI):[M − H]− = 553. |

### Example 5.1

### Preparation of 2-(3,3-Dioxo-2,3-dihydro-3λ⁶-benzo[1,3]oxathiol-5-yloxy)-4-fluoro-6-(2-fluoro-4-iodo-phenylamino)-benzamide

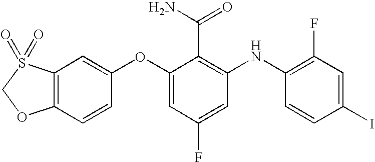

2-(3,3-Dioxo-2,3-dihydro-3λ⁶-benzo[1,3]oxathiol-5-yloxy)-4-fluoro-6-(2-fluoro-4-iodo-phenylamino)-benzamide was prepared by applying the general procedures described above in 28% yield.

MS (ESI): [M+H]⁺=559.

The following example compounds 5.2 to 5.18 were prepared by applying the described procedures above:

| Example | Structure | Name | Analytical data |
|---|---|---|---|
| 5.2 | 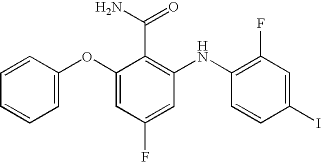 | 4-Fluoro-2-(2-fluoro-4-iodo-phenylamino)-6-phenoxy-benzamide | MS (ESI):[M + H]⁺ = 467 |
| 5.3 | 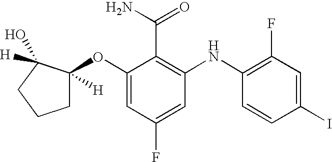 | 4-Fluoro-2-(2-fluoro-4-iodo-phenylamino)-6-((1S,2S)-2-hydroxy-cyclopentyloxy)-benzamide | MS (ESI):[M + H]⁺ = 475 |
| 5.4 | 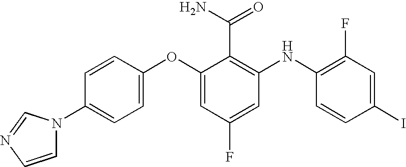 | 4-Fluoro-2-(2-fluoro-4-iodo-phenylamino)-6-(4-imidazol-1-yl-phenoxy)-benzamide | MS (ESI):[M + H]⁺ = 533 |
| 5.5 | 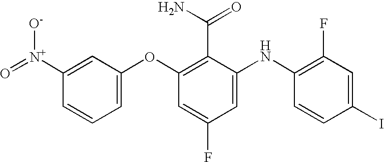 | 4-Fluoro-2-(2-fluoro-4-iodo-phenylamino)-6-(3-nitro-phenoxy)-benzamide | MS (ESI):[M + H]⁺ = 512 |
| 5.6 | 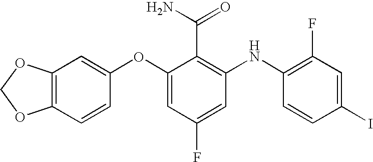 | 2-(Benzo[1,3]dioxol-5-yloxy)-4-fluoro-6-(2-fluoro-4-iodo-phenylamino)-benzamide | MS (ESI):[M + H]⁺ = 511 |
| 5.7 | 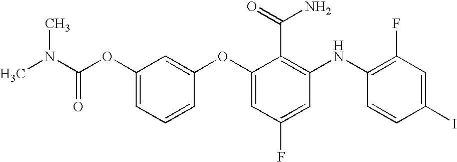 | Dimethyl-carbamicacid 3-[2-carbamoyl-5-fluoro-3-(2-fluoro-4-iodo-phenylamino)-phenoxy]-phenylester | MS (ESI):[M + H]⁺ = 554 |
| 5.8 | 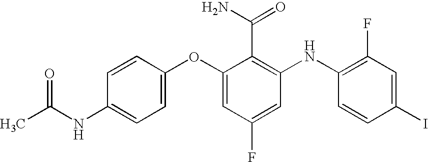 | 2-(4-Acetylamino-phenoxy)-4-fluoro-6-(2-fluoro-4-iodo-phenylamino)-benzamide | MS (ESI):[M + H]⁺ = 524 |
| 5.9 | 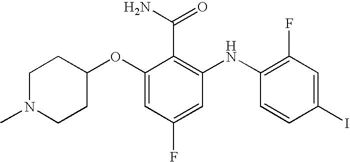 | 4-Fluoro-2-(2-fluoro-4-iodo-phenylamino)-6-(1-methyl-piperidin-4-yloxy)-benzamide | ¹H-NMR:(d6-DMSO, 300 MHz)9.65 (s, 1 H); 7.80 (br. s, 1H); 7.63 (dd, 1 H); 7.57 (br. s,1 H); 7.45 (d, 1 H); 7.17 (t, 1H); 6.54 (dd, 1 H); 6.38 (dd, 1H); 4.42-4.52 (m, 1 H); 2 Hobscured by solvent signal;2.11-2.23 (m, 5 H); 1.84-1.93 (m, 2 H); 1.62-1.73 (m,2 H).MS (ESI):[M + H]⁺ = 488. |
| 5.10 | 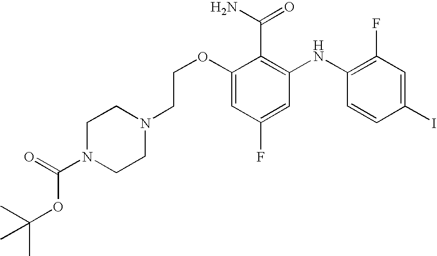 | 4-{2-[2-Carbamoyl-5-fluoro-3-(2-fluoro-4-iodo-phenylamino)-phenoxy]-ethyl}-piperazine-1-carboxylic acid tert-butyl ester | ¹H-NMR:(d6-DMSO, 400 MHz)10.68 (s, 1 H); 8.44 (br. s, 1H); 7.77 (br. s, 1 H); 7.65 (dd,1 H); 7.47 (d, 1 H); 7.22 (t, 1H); 6.50 (dd, 1 H); 6.39 (dd, 1H); 4.19 (t, 1 H); 3.25-3.28(m, 4 H); 2.66 (t, 2 H); 2.34-2.38 (m, 4 H); 1.36 (s, 9 H).MS (ESI):[M + H]⁺ = 603. |
| 5.11 | 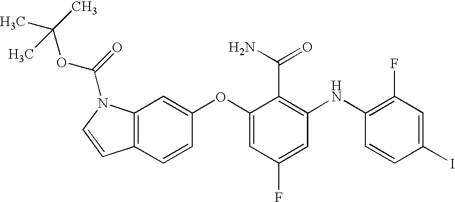 | 6-[2-Carbamoyl-5-fluoro-3-(2-fluoro-4-iodo-phenylamino)-phenoxy]-indole-1-carboxylic acid tert-butyl ester | MS (ESI):[M + H]⁺ = 606 |
| 5.12 | 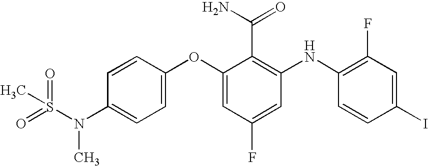 | 4-Fluoro-2-(2-fluoro-4-iodo-phenylamino)-6-[4-(methanesulfonyl-methyl-amino)-phenoxy]-benzamide | MS (ESI):[M + H]⁺ = 574 |
| 5.13 | 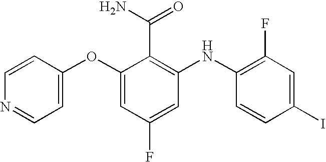 | 4-Fluoro-2-(2-fluoro-4-iodo-phenylamino)-6-(pyridin-4-yloxy)-benzamide | MS (ESI):[M + H]⁺ = 468 |
| 5.14 | 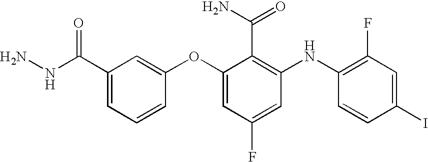 | 4-Fluoro-2-(2-fluoro-4-iodo-phenylamino)-6-(3-hydrazinocarbonyl-phenoxy)-benzamide | MS (ESI):[M + H]⁺ = 525 |
| 5.15 | 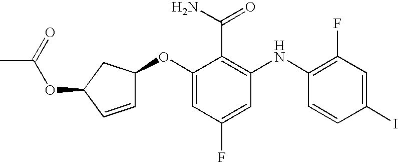 | Acetic acid (1S,4R)-4-[2-carbamoyl-5-fluoro-3-(2-fluoro-4-iodo-phenylamino)-phenoxy]-cyclopent-2-enyl ester | MS (ESI):[M + H]⁺ = 473. |
| 5.16 | 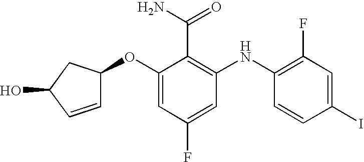 | 4-Fluoro-2-(2-fluoro-4-iodo-phenylamino)-6-((1R,4S)-4-hydroxy-cyclopent-2-enyloxy)-benzamide | MS (ESI):[M + H]⁺ = 515. |
| 5.17 | 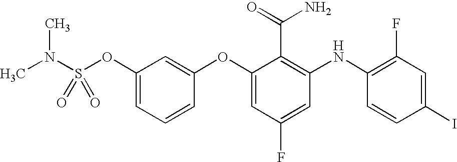 | Dimethyl-sulfamicacid 3-[2-carbamoyl-5-fluoro-3-(2-fluoro-4-iodo-phenylamino)-phenoxy]-phenyl | MS (ESI):[M + H]⁺ = 590. |
| 5.18 | 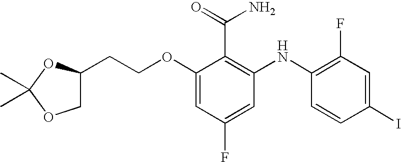 | 2-[2-((S)-2,2-Dimethyl-[1,3]dioxolan-4-yl)-ethoxy]-4-fluoro-6-(2-fluoro-4-iodo-phenylamino)-benzamide | MS (ESI):[M + H]⁺ = 519 |

### Example Compound 6.1a

### Preparation of 4-Fluoro-2-(2-fluoro-4-iodo-phenylamino)-6-(3-methanesulfonylamino-phenoxy)-benzamide

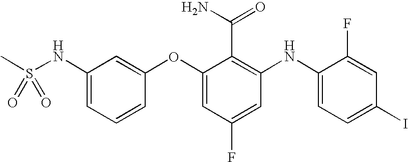

In analogy to GP 6, 241 mg of 2-(3-Amino-phenoxy)-4-fluoro-6-(2-fluoro-4-iodo-phenylamino)-benzamide (0.5 mmol, 1 eq.) were suspended in dichloromethane and 48 μL of pyridine (0.6 mmol, 1.2 eq.) were added to form a clear solution. The mixture was cooled to 3° C. for 10 Min before 41 μL of methyl sulfonyl chloride (0.53 mmol, 1.05 eq.) were added. The mixture was treated with another 0.3 eq. of reactants. The reaction mixture was washed with aqueous half concentrated sodium bicarbonate solution one time and the aqueous layer extracted twice with methylene chloride. The combined organic layers were dried by passing over a silicone filter pad and concentrated to afford 327 mg of crude product. The concentrate was purified (FlashMaster column chromatography, hexane/ethyl acetate 99-30%) to afford 170 mg (61% yield, 0.3 mmol) of the desired product.

¹H-NMR: (d6-DMSO, 300 MHz) 9.89 (s, 1H); 9.02 (s, 1H); 7.87 (sbr, 1H); 7.84 (sbr, 1H); 7.66 (dd, 1H); 7.47 (dbr, 1H); 7.32 (dd, 1H); 7.21 (dd, 1H); 6.98 (dbr, 1H); 6.94 (dd, 1H); 6.76 (dd, 1H); 6.56 (dbr, 1H); 6.16 (dd, 1H); 3.00 (s, 3H).

MS (ESI): [M+H]⁺=560

In addition to example compound 6.1a, example compound 6.1b was isolated:

### Example Compound 6.1b

### 4-Fluoro-2-(2-fluoro-4-iodo-phenylamino)-6-(3-bis-methanesulfonyl-amino-phenoxy)-benzamide

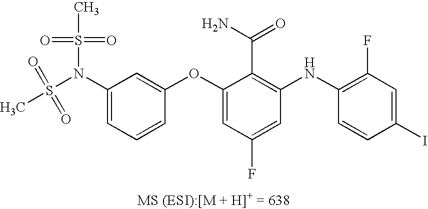

### Example Compound 6.2

### Preparation of 2-{2-[2-Carbamoyl-5-fluoro-3-(2-fluoro-4-iodo-phenylamino)-phenoxy]-ethyl}-piperidine-1-carboxylic acid dimethylamide

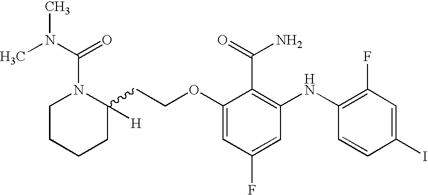

In analogy to GP 7, 150 mg of 4-fluoro-2-(2-fluoro-4-iodo-phenylamino)-6-(2-piperidin-2-yl-ethoxy)-benzamide (0.3 mmol) were dissolved in 4.5 mL DMF and treated subsequently with 50 μL triethylamine (1.2 eq.) and 33 μL dimethylcarbamoyl chloride (1.2 eq.). The reaction mixture was stirred at rt for 16 h, quenched with water, extracted with DCM, the combined organic layers were dried and concentrated in vacuo. Flash column chromatography provided the target compound.

¹H-NMR: (d6-DMSO, 400 MHz): 9.70 (s, 1H); 7.77 (s, 1H); 7.72 (s, 1H); 7.63 (dd, 1H); 7.44 (d, 1H); 7.18 (t, 1H); 6.41 (d, 2H); 3.93-4.02 (m, 2H); 3.85-3.92 (m, 1H); 2.92 (t, 1H); 2.59 (s, 6H); 2.11-2.19 (m, 1H); 1.85-1.94 (m, 1H); 1.48-1.62 (m, 5H); 1.26-1.36 (m, 1H). [one proton obscured by solvent signal]

MS (ESI): [M+H]⁺=573.

### Example Compound 6.3

### Preparation of 2-[3-[[(propylamino)sulfonyl]amino]phenoxy]-4-fluoro-6-[(2-fluoro-4-iodophenyl)amino]-benzamide

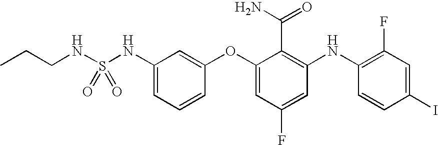

In analogy to GP 5, 422 mg of 2-(3-Amino-phenoxy)-4-fluoro-6-(2-fluoro-4-iodo-phenylamino)-benzamide (0.88 mmol; 1 eq.) were dissolved in 17.54 mL of DCM and treated subsequently with 180 μL N-Ethyl-N,N-diisopropyl amin (1.05 mmol; 1.2 eq.). The solution was cooled to 0° C. for 60 Min, treated with 152.04 mg propyl sulfamoyl chloride (0.96 mmol; 1.1 eq.) and stirred for 30 Min at 0° C. and 3 h at RT. Since the reaction was not completed another 0.3 eq. N-Ethyl-N,N-diisopropyl amin and 0.2 eq. propyl sulfamoyl chloride were added and the mixture stirred at RT for 48 h. The suspension was filtered off and the white crystals were washed with DCM and dried to afford 469 mg of the pure target compound (89% yield, 0.78 mmol).

¹H-NMR: (d6-DMSO, 300 MHz)

MS (ESI): [M+H]⁺=603

### Example Compound 6.4

### Preparation of 2-(3-Acetylamino-phenoxy)-4-fluoro-6-(2-fluoro-4-iodo-phenylamino)-benzamide

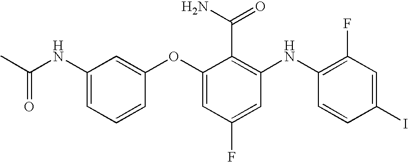

In analogy to GP 8, 96.25 mg of 2-(3-Amino-phenoxy)-4-fluoro-6-(2-fluoro-4-iodo-phenylamino)-benzamide (0.2 mmol; 1 eq.) were dissolved in 5 mL of DCM, treated with 41.08 μL N-Ethyl-N,N-diisopropyl amin (0.24 mmol; 1.2 eq.). Upon cooling to 0° C., 0.014 ml of acetyl chloride (0.2 mmol; 1.01 eq.) were added the mixture stirred at 3° C. for 1 h and at RT for 23 h. The suspension was filtered off and the precipitate was washed with DCM and dried to afford 65 mg of the pure target compound (62% yield, 0.12 mmol).

¹H-NMR: (d6-DMSO, 300 MHz)

MS (ESI): [M+H]⁺=603

The following example compounds 6.5 to 6.30 were prepared in analogy to example compounds 6.1a to 6.4 by applying GP 5 (for sulfamides), GP 6 (for sulfonamides), GP 7 (for ureas) or GP8 (for amides) to the respective amines.

| Ex- | | | |
|---|---|---|---|
| ample | Structure | Name | Analytical data |
| 6.5 | 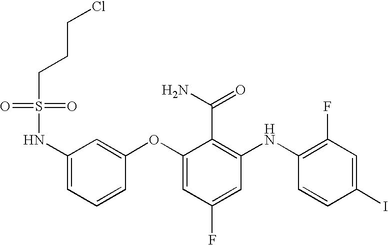 | 2-[3-(3-Chloro-propane-1-sulfonylamino)-phenoxy]-4-fluoro-6-(2-fluoro-4-iodo-phenylamino)-benzamide | MS (ESI):[M + H]⁺ = 622 |
| 6.6 | 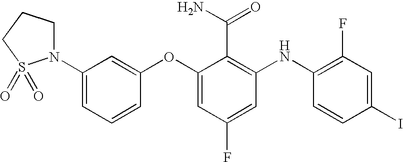 | 2-[3-(1,1-Dioxo-1λ⁶-isothiazolidin-2-yl)-phenoxy]-4-fluoro-6-(2-fluoro-4-iodo-phenylamino)-benzamide | ¹H-NMR:(d6-DMSO, 300 MHz)9.01 (s, 1 H); 7.87 (sbr, 1 H);7.86(sbr, 1 H); 7.66 (dd, 1H); 7.47 (dbr, 1 H); 7.37 (dd,1 H); 7.21 (dd, 1 H); 7.04-6.96 (m, 2 H); 6.77 (dd, 1 H);6.54 (dbr, 1 H); 6.04 (dd, 1H); 3.71 (t, 2 H); 3.50 (t, 2H); 2.36 (tt, 2 H).MS (ESI):[M + H]⁺ = 586 |
| 6.7 | 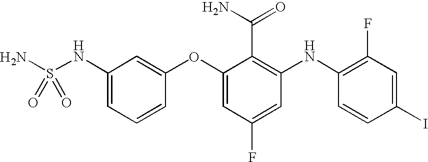 | 2-[3-[[(amino)sulfonyl]amino]phenoxy]-4-fluoro-6-[(2-fluoro-4-iodophenyl)amino]-benzamide | MS (ESI):[M + H]⁺ = 561 |
| 6.8 | 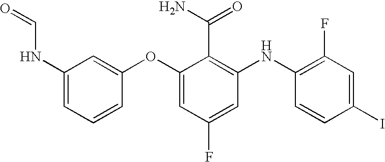 | 4-Fluoro-2-(2-fluoro-4-iodo-phenylamino)-6-(3-formylamino-phenoxy)-benzamide | MS (ESI):[M + H]⁺ = 510 |
| 6.9 | 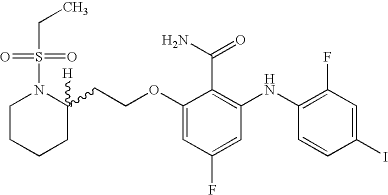 | 2-[2-(1-Ethanesulfonyl-piperidin-2-yl)-ethoxy]-4-fluoro-6-(2-fluoro-4-iodo-phenylamino)-benzamide | ¹H-NMR:(d6-DMSO, 300 MHz)9.73 (s, 1 H); 7.81 (s, 1 H);7.68 (dd, 1 H); 7.62 (s, 1 H);7.49 (d, 1 H); 7.23 (t, 1 H);6.44-6.50 (m, 2 H); 3.98-4.13 (m, 3 H); 3.51-3.58 (m,1 H); 3.00-3.12 (m, 3 H);2.18-2.30 (m, 1 H); 1.97-2.07 (m, 1 H); 1.55-1.72 (m,5 H); 1.36-1.52 (m, 1 H);1.18 (t, 3 H). |
| 6.10 | 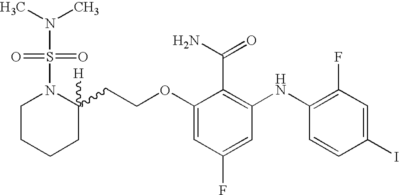 | 2-[2-(1-Dimethylsulfamoyl-piperidin-2-yl)-ethoxy]-4-fluoro-6-(2-fluoro-4-iodo-phenylamino)-benzamide | ¹H-NMR(d6-DMSO, 400 MHz):9.74 (s, 1 H); 7.79 (s, 1 H);7.63 (dd, 1 H); 7.58 (d, 1 H);7.44 (d, 1 H); 7.18 (t, 1 H);6.39-6.44 (m, 2 H); 3.89-4.07 (m, 2 H); 3.87-3.94 (m,1 H); 3.33-3.40 (m, 1 H);2.99 (t, 1 H); 2.62 (s, 6 H);2.12-2.19 (m, 1 H); 1.97-2.07 (m, 1 H); 1.49-1.71 (m,5 H); 1.34-1.48 (m, 1 H). |
| 6.11 | 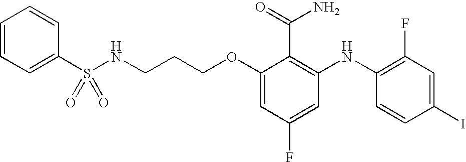 | 2-(3-Benzene-sulfonylamino-propoxy)-4-fluoro-6-(2-fluoro-4-iodo-phenylamino)-benzamide | ¹H-NMR:(d6-DMSO, 300 MHz)9.47 (s, 1 H); 7.56-7.83 (m,9 H); 7.49 (d, 1 H); 7.21 (t, 1H); 6.46 (s, 1 H); 6.42 (s, 1H); 4.03 (t, 2 H); 2.93 (q, 2H); 1.85 (m, 2 H).MS (ESI):[M + H]⁺ = 588. |
| 6.12 | 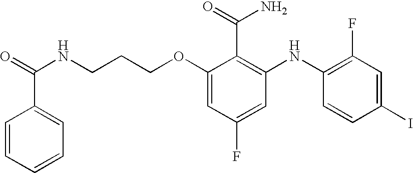 | 2-(3-Benzoylamino-propoxy)-4-fluoro-6-(2-fluoro-4-iodo-phenylamino)-benzamide | ¹H-NMR:(d6-DMSO, 300 MHz)9.61 (s, 1 H); 8.59 (t, 1 H);7.81-7.89 (m,4 H); 7.68 (dd,1 H); 7.44-7.56 (m, 2 H);7.23 (t, 1 H);6.44-6.54 (m,2 H); 4.08 (t, 2H); 3.47 (q, 2H); 2.01 (m, 2 H).MS (ESI):[M + H]⁺ = 552. |
| 6.13 | 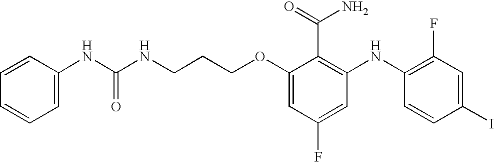 | 4-Fluoro-2-(2-fluoro-4-iodo-phenylamino)-6-[3-(3-phenyl-ureido)-propoxy]-benzamide | MS (ESI):[M + H]⁺ = 567. |
| 6.14 | 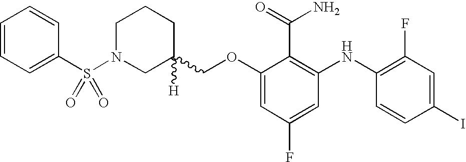 | 2-(1-Benzenesulfonyl-piperidin-3-ylmethoxy)-4-fluoro-6-(2-fluoro-4-iodo-phenylamino)-benzamide | ¹H-NMR:(d6-DMSO, 300 MHz)9.33 (s, 1 H); 7.79 (br. s, 1H); 7.57-7.73 (m, 7 H); 7.45(d, 1 H); 7.16 (t, 1 H); 6.38-6.49 (m, 2 H); 3.85-3.91 (m,2 H); 3.59-3.64 (m, 1 H);3.41-3.47 (m, 1 H); 2.00-2.37 (m, 3 H); 1.62-1.74 (m,2 H); 1.39-1.54 (m, 1 H);0.98-1.12 (m, 1 H);MS (ESI):[M + H]⁺ = 628. |
| 6.15 | 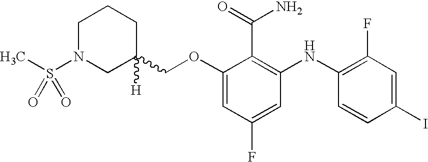 | 4-Fluoro-2-(2-fluoro-4-iodo-phenylamino)-6-(1-methanesulfonyl-piperidin-3-ylmethoxy)-benzamide | MS (ESI):[M + H]⁺ = 566. |
| 6.16 | 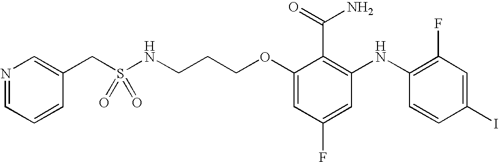 | 4-Fluoro-2-(2-fluoro-4-iodo-phenylamino)-6-[3-(pyridin-3-ylmethanesulfonylami-no)-propoxy]-benzamide | MS (ESI):[M + H]⁺ = 603. |
| 6.17 | 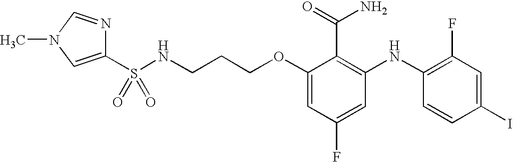 | 4-Fluoro-2-(2-fluoro-4-iodo-phenylamino)-6-[3-(1-methyl-1H-imidazole-4-sulfonylamino)-propoxy]-benzamide | MS (ESI):[M + H]⁺ = 592. |
| 6.18 | 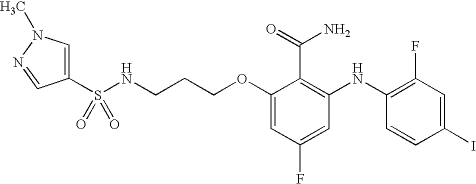 | 4-Fluoro-2-(2-fluoro-4-iodo-phenylamino)-6-[3-(1-methyl-1H-pyrazole-4-sulfonylamino)-propoxy]-benzamide | MS (ESI):[M + H]⁺ = 592. |
| 6.19 | 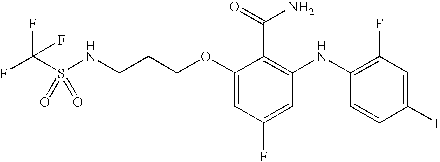 | 4-Fluoro-2-(2-fluoro-4-iodo-phenylamino)-6-(3-trifluoromethanesulfonylamino-propoxy)-benzamide | MS (ESI):[M + H]⁺ = 580. |
| 6.20 | 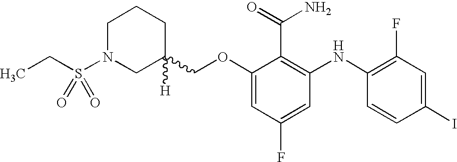 | 2-(1-Ethanesulfonyl-piperidin-3-ylmethoxy)-4-fluoro-6-(2-fluoro-4-iodo-phenylamino)-benzamide | MS (ESI):[M + H]⁺ = 580. |
| 6.21 | 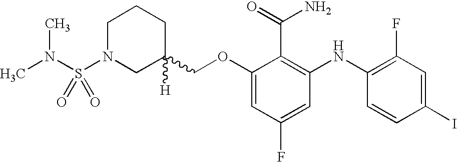 | 2-(1-Dimethylsulfamoyl-piperidin-3-ylmethoxy)-4-fluoro-6-(2-fluoro-4-iodo-phenylamino)-benzamide | ¹H-NMR:(d6-DMSO, 400 MHz)9.40 (s, 1 H); 7.81 (br. s, 1H); 7.61-7.64 (m, 2 H); 7.45(d, 1 H); 7.17 (t, 1 H); 6.49(dd, 1 H); 6.41 (d, 1 H); 3.89-3.95 (m, 2 H); 3.53-3.58 (m,1 H); 3.38-3.44 (m, 1 H);2.68-2.85 (m, 8 H); 2.00-2.09 (m ,1 H); 1.66-1.79 (m,2 H); 1.40-1.52 (m, 1 H);1.18-1.28 (m, 1 H).MS (ESI):[M + H]⁺ = 595. |
| 6.22 | 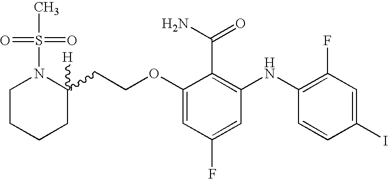 | 4-Fluoro-2-(2-fluoro-4-iodo-phenylamino)-6-[2-(1-methanesulfonyl-piperidin-2-yl)-ethoxy]-benzamide | ¹H-NMR:(d6-DMSO, 400 MHz)9.68 (s, 1 H); 7.76 (br. s, 1H); 7.63 (dd, 1 H); 7.55 (br. s,1 H); 7.44 (d, 1 H); 7.19 (t, 1H); 6.40-6.44 (m, 2 H); 3.95-4.05 (m, 3 H);3.56 (br. d,1 H); 3.01 (br. t,1 H); 2.91 (s,3 H); 2.17-2.26 (m, 1 H);1.87-1.96 (m, 1 H); 1.37-1.71 (m, 6 H).MS (ESI):[M + H]⁺ = 580. |
| 6.23 | 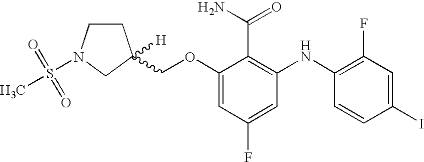 | 4-Fluoro-2-(2-fluoro-4-iodo-phenylamino)-6-(1-methanesulfonyl-pyrrolidin-3-ylmethoxy)-benzamide | MS ESI):[M + H]⁺ = 552. |
| 6.24 | 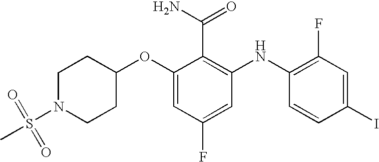 | 4-Fluoro-2-(2-fluoro-4-iodo-phenylamino)-6-(1-methanesulfonyl-piperidin-4-yloxy)-benzamide | ¹H-NMR:(d6-DMSO, 300 MHz)9.20 (s, 1 H); 7.79 (br. s, 1H); 7.63 (dd, 1 H); 7.59 (br. s,1 H); 7.44 (d, 1 H); 7.16 (t, 1H); 6.59 (dd, 1 H);6.40 (dd, 1H); 4.61-4.67 (m,1 H); 3.20-3.30 (m, 2 H); 3.08-3.17(m, 2 H);2.85 (s, 3 H); 1.91-2.01 (m, 2 H); 1.75-1.86 (m,2 H).MS (ESI):[M + H]⁺ = 552. |
| 6.25 | 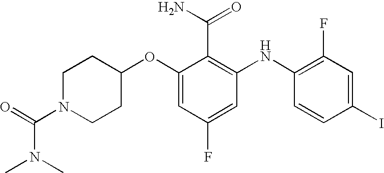 | 4-[2-Carbamoyl-5-fluoro-3-(2-fluoro-4-iodo-phenylamino)-phenoxy]-piperidine-1-carboxylic aciddimethylamide | ¹H-NMR:(d6-DMSO, 400 MHz)9.46 (s, 1 H); 7.78 (br. s, 1H); 7.63 (dd, 1 H); 7.58 (br. s,1 H); 7.44 (d, 1 H); 7.17 (t, 1H); 6.59 (dd, 1 H); 6.39 (dd, 1H); 4.60-4.67 (m, 1 H); 2. Hobscured by solvent signal;2.95-3.06 (m, 2 H); 2.70 (s,6 H); 1.86-1.94 (m, 2 H);1.60-1.69 (m, 2 H).MS (ESI):[M + H]⁺ = 545. |
| 6.26 | 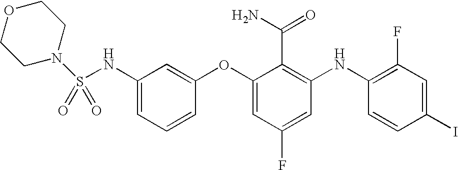 | 4-Fluoro-2-(2-fluoro-4-iodo-phenylamino)-6-[3-(morpholine-4-sulfonylamino)-phenoxy]-benzamide | MS (ESI):[M + H]⁺ = 631. |
| 6.27 | 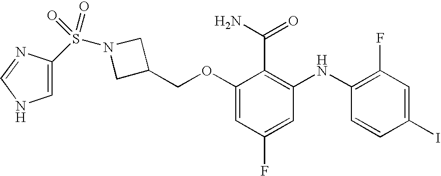 | 4-Fluoro-2-(2-fluoro-4-iodo-phenylamino)-6-[1-(1H-imidaozle-4-sulfonyl)-azetidin-3-ylmethoxy]-benzamide | MS (ESI):[M + H]⁺ = 590. |
| 6.29 | 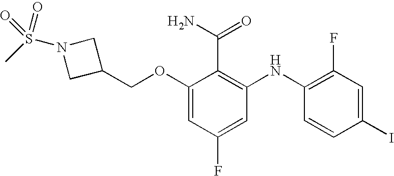 | 4-Fluoro-2-(2-fluoro-4-iodo-phenylamino)-6-(1-methanesulfonyl-azetidin-3-ylmethoxy)-benzamide | MS (ESI):[M + H]⁺ = 538. |
| 6.30 | 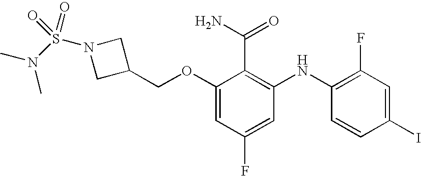 | 2-(1-Dimethylsulfamoyl-azetidin-3-ylmethoxy)-4-fluoro-6-(2-fluoro-4-iodo-phenylamino)-benzamide | MS (ESI):[M + H]⁺ = 567. |

### Example Compound 7.1

### Preparation of 2-(3,4-Dihydroxy-4-methyl-pentyloxy)-4-fluoro-6-(2-fluoro-4-iodo-phe-nylamino)-benzamide

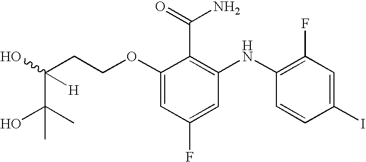

In analogy to GP 13, 35 mg of 4-Fluoro-2-(2-fluoro-4-iodo-phenylamino)-6-(4-methyl-pent-3-enyloxy)-benzamide (0.074 mmol, 1 eq.) were dissolved in Acetone and 0.75 ml of water were added to form a suspension. 19 mg N-methyl-morpholino-N-oxide (0.14 mmol, 1.9 eq.) were added and the mixture cooled to +3° C. 10 μl of an Osmiumtetroxide solution (2.5 weight % in tert.-butanol) were added and the mixture stirred for 40 Min in an ice bath and then for 20 h at rt. The reaction mixture was concentrated, 10 ml of water and ethyl acetate were added and the organic layer was extracted three times with ethyl acetate. The organic layer was washed one time with brine, dried over sodium sulfate, filtered off and concentrated to afford 39 mg of crude product which required no further purification.

¹H-NMR: (d6-DMSO, 300 MHz): 10.05 (s, 1H); 7.78 (sbr, 1H); 7.73 (sbr, 1H); 7.63 (dd, 1H); 7.45 (ddd, 1H); 7.19 (dd, 1H); 6.45 (dd, 2H); 4.63 (d, 1H); 4.16 (s, 1H); 4.13 (dd, 2H); 3.35-3.25 (m, 1H); 2.04 (m, 1H); 1.58 (m, 1H); 1.05 (s, 3H); 1.00 (s, 3H).

### MS (ESI): [M+H]⁺=516

The following example compounds 7.2 to 7.10 were prepared in analogy to example compound 7.1 and GP 13 from the respective olefins.

| Example | Structure | Name | Analytical data |
|---|---|---|---|
| 7.2 | 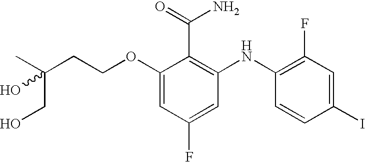 | 2-(3,4-Dihydroxy-3-methyl-butoxy)-4-fluoro-6-(2-fluoro-4-iodo-phenylamino)-benzamide | ¹H-NMR:(d6-DMSO, 300 MHz):10.12 (s, 1 H); 7.92 (sbr, 1 H);7.69 (sbr, 1 H); 7.63 (dd, 1 H);7.45 (dd, 1 H); 7.19 (dd, 1 H);6.47 (dd, 1 H); 6.39 (dd, 1 H);4.67 (dd, 1 H); 4.40 (s, 1 H);4.14 (dd, 2 H); 3.18 (m, 2 H);1.85 (m, 2 H); 1.06 (s,3 H).MS (ESI):[M + H]⁺ = 493 |
| 7.3 | 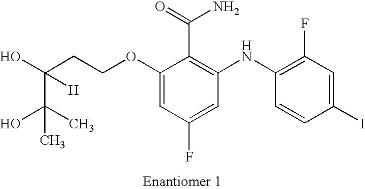 | 2-(3,4-Dihydroxy-4-methyl-pentyloxy)-4-fluoro-6-(2-fluoro-4-iodo-phenylamino)-benzamide | MS (ESI):[M + H]⁺ = 516Optical rotation: −46.9 grd |
| 7.4 | 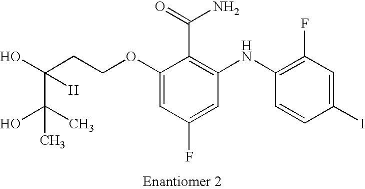 | 2-(3,4-Dihydroxy-4-methyl-pentyloxy)-4-fluoro-6-(2-fluoro-4-iodo-phenylamino)-benzamide | MS (ESI):[M + H]⁺ = 516Optical rotation: +40.5 grd |
| 7.5 | 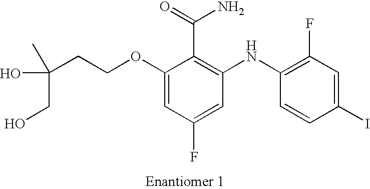 | 2-(3,4-Dihydroxy-3-methyl-butoxy)-4-fluoro-6-(2-fluoro-4-iodo-phenylamino)-benzamide | ¹H-NMR: (d6-DMSO, 300 MHz):10.12 (s, 1 H); 7.92 (sbr, 1 H);7.69 (sbr 1 H); 7.63 (dd, 1 H);7.45 (dd, 1 H); 7.19 (dd, 1 H);6.47 (dd, 1 H); 6.39 (dd, 1 H);4.67 (dd, 1 H); 4.40 (s, 1 H);4.14 (dd, 2 H); 3.18 (m, 2 H);1.85 (m, 2 H); 1.06 (s,3 H).MS (ESI): [M + H]⁺ = 493 |
| 7.6 | 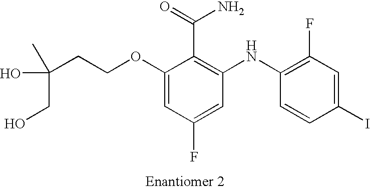 | 2-(3,4-Dihydroxy-3-methyl-butoxy)-4-fluoro-6-(2-fluoro-4-iodo-phenylamino)-benzamide | ¹H-NMR: (d6-DMSO, 300 MHz):10.12 (s, 1 H); 7.92 (sbr, 1 H);7.69 (sbr, 1 H); 7.63 (dd, 1 H);7.45 (dd, 1 H); 7.19 (dd, 1 H);6.47 (dd, 1 H); 6.39 (dd, 1 H);4.67 (dd, 1 H); 4.40 (s, 1 H);4.14 (dd, 2 H); 3.18 (m, 2 H);1.85 (m, 2 H); 1.06 (s,3 H).MS (ESI): [M + H]⁺ = 493. |
| 7.7 | 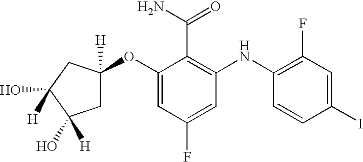 | 2-((1S,3S,4R)-3,4-Dihydroxy-cyclopentyloxy)-4-fluoro-4-(2-fluoro-6-iodo-phenylamino)-benzamide | MS (ESI): [M + H]⁺= 491 |
| 7.8 | 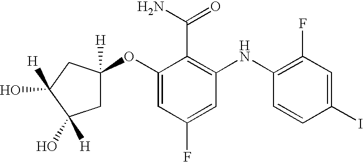 | 2-((1S,3S,4R)-3,4-Dihydroxy-cyclopentyloxy)-4-fluoro-4-(2-fluoro-6-iodo-phenylamino)-benzamide | MS (ESI): [M + H]⁺= 491. |
| 7.9 | 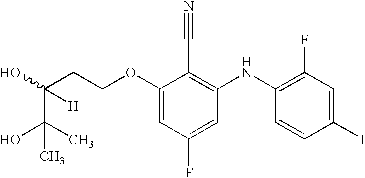 | 2-(3,4-Dihydroxy-4-methyl-pentyloxy)-4-fluoro-6-(2-fluoro-4-iodo-phenylamino)-benzonitrile | MS (ESI): [M + H]⁺ = 489. |
| 7.10 | 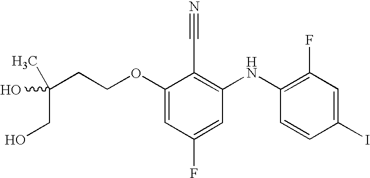 | 2-(3,4-Dihydroxy-3-methyl-butoxy)-4-fluoro-6-(2-fluoro-4-iodo-phenylamino)-benzonitrile | MS (ESI): [M + H]⁺ = 475. |

### Example Compound 8.1

### Preparation of 2-((S)-3,4-Dihydroxy-butoxy)-4-fluoro-6-(2-fluoro-4-iodo-phenylamino)-benzamide

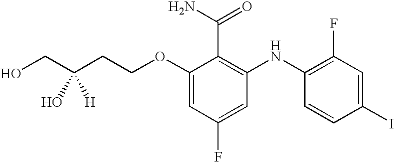

In analogy to GP 4b, 2-{2-[(4S)-2,2-Dimethyl-1,3-dioxolan-4-yl]ethoxy}-4-fluoro-6-[(2-fluoro-4-iodophenyl)amino]benzamide (38 mg, 0.73 mmol) was dissolved in THF (2 ml). 1 ml of hydrochloric acid (aq.; 37%) was added, and the solution was stirred for 16 h at rt. The mixture was concentrated in vacuo and the remaining solid was purified by preparative HPLC to afford 22 mg product (61% yield; 0.45 mmol).

¹H-NMR: (d6-DMSO, 400 MHz): 10.06 (s, 1H, NH), 7.75 (s, 1H, NH₂), 7.84 (s, 1H, NH₂), 7.67 (dd, 1H), 7.49 (d, 1H), 7.22 (t, 1H), 6.50 (dd, 1H), 6.43 (d, 1H), 4.75 (d, 1H, OH), 4.60 (t, 1H, OH), 4.12-4.21 (m, 2H), 3.59-3.67 (m, 1H), 3.25-3.40 (m, under DMSO-signal), 1.93-2.03 (m, 1H, 1.63-1.74 (m, 1H).

MS (ESI): [M+H]⁺=479.

The following example compounds 8.2 to 8.6 were prepared in analogy to the afore described procedures by acetonide cleavage of the respective precursor compounds.

| Example | Structure | Name | Analytical data |
|---|---|---|---|
| 8.2 | 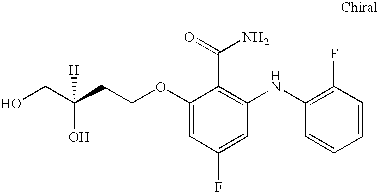 | 2-((R)-3,4-Dihydroxy-butoxy)-4-fluoro-6-(2-fluoro-phenylamino)-benzamide | MS (ESI): [M + H]⁺ = 352. |
| 8.3 | 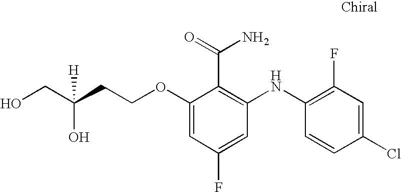 | 2-(4-Chloro-2-fluoro-phenylamino)-6-((R)-3,4-dihydroxy-butoxy)-4-fluoro-benzamide | MS (ESI): [M + H]⁺ = 387. |
| 8.4 | 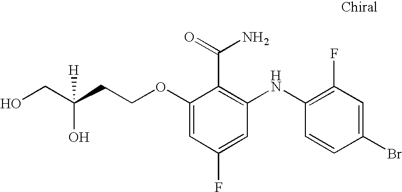 | 2-(4-Bromo-2-fluoro-phenylamino)-6-((R)-3,4-dihydroxy-butoxy)-4-fluoro-benzamide | ¹H-NMR(d₆-DMSO; 400 MHz):10.04 (s, 1 H); 7.82 (s, 1 H);7.72 (s, 1 H); 7.54-7.57 (m,1 H); 7.29-7.36 (m, 2 H);6.46 (dd, 1 H); 6.38 (d, 1 H);4.72 (d, 1 H); 4.57 (t, 1 H);4.07-4.28 (m, 2 H); 3.56-3.64(m, 1 H); 3.22-3.36 (m,2 H); 1.91-1.99 (m, 1 H);1.61-1.70 (m, 1 H).MS (ESI): [M + H]⁺ = 431/433 (Brisotope pattern) |
| 8.5 | 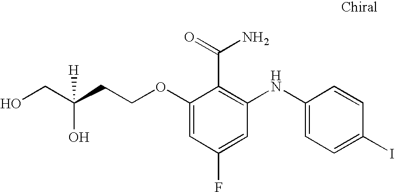 | 2-((R)-3,4-Dihydroxy-butoxy)-4-fluoro-6-(4-iodo-phenylamino)-benzamide | ¹H-NMR(d₆-DMSO; 400 MHz):9.71 (s, 1 H); 7.75 (s, 1 H);7.63 (s, 1 H); 7.57 (d, 2 H);6.93 (d, 2 H); 6.49 (dd, 1 H);6.42 (dd, 1 H); 4.69 (d, 1 H);4.56 (t, 1 H); 4.07-4.16 (m,2 H); 3.56-3.64 (m, 1 H);3.22-3.36 (m, 2 H); 1.89-1.97(m, 1 H); 1.60-1.69 (m,1 H).MS (ESI): [M + H]⁺ = 461. |
| 8.6 | 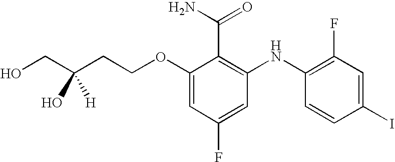 | 2-((R)-3,4-Dihydroxy-butoxy)-4-fluoro-6-(2-fluoro-4-iodo-phenylamino)-benzamide | ¹H-NMR(d₆-DMSO; 400 MHz):10.02 (s, 1 H); 7.81 (sbr, 1 H);7.71 (sbr, 1 H); 7.63 (dd, 1 H);7.45 (dbr, 1 H); 7.18 (dd,1 H); 7.46 (dd, 1 H); 6.39(dbr, 1 H); 4.13 (m, 2 H); 3.60(m, 2 H); 3.39-3.21 (m, 2 H);1.94 (m, 1 H); 1.65 (m, 1 H).MS (ESI): [M + H]⁺ = 479. |

### Example Compound 9.1

### Preparation of 2-((R)-3,4-Dihydroxy-butoxy)-6-(4-ethynyl-2-fluoro-phenylamino)-4-fluoro-benzamide

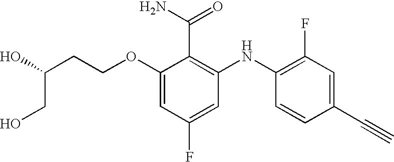

### Step A:

In analogy to GP 11a, 71.73 mg of 2-((R)-3,4-Dihydroxy-butoxy)-4-fluoro-6-(2-fluoro-4-iodo-phenylamino)-benzamide (0.15 mmol; 1 eq.), 3.45 mg bis[(1,2,4,5-eta)-1,5-diphenyl-1,4-pentadien-3-one]-palladium (0.006 mmol; 0.004 eq.), 1.14 mg copper(I) iodide (0.006 mmol; 0.004 eq.); 7.87 mg triphenylphosphine (0.03 mmol, 0.2 eq.) were mixed with 1.5 ml of triethyl amine in a pressure tube. Upon flushing three times with N₂, 88.4 mg of trimethylsilyl acetylene (0.9 mmol; 6 eq.) were added, the pressure tube was sealed and the resulting suspension was stirred vigorously at 60° C. for 3 h. The mixture was concentrated, redissolved in hexane/ethyl acetate 1:1 and filtered over a NH₂-column (hexane/ethyl acetate 50:50 to 0:100 to pure methanol). The filtrate was concentrated to afford 58.17 mg (86.46% yield, 0.13 mmol) of the silylated ethynyl compound.

### Step B:

In analogy to GP 12, 52.72 mg of 2-((R)-3,4-Dihydroxy-butoxy)-4-fluoro-6-(2-fluoro-4-trimethylsilanylethynyl-phenylamino)-benzamide (0.12 mmol, 1 eq.) were dissolved in 1 ml THF, then 0.12 ml of TBAF-solution (1 M in THF; 0.12 mmol; 1 eq.) was added and the mixture stirred at RT for 90 Min under nitrogen. The crude mixture was partitioned between 5 ml of water and 10 ml of ethyl acetate and the aqueous phase was extracted twice with ethyl acetate (10 ml each). The combined organic layers were washed once with half concentrated brine, dried over sodium sulfate, filtered off and concentrated to afford 44.63 mg of crude product. The concentrate was suspended in DCM, stirred at RT for 1 h, filtered off and washed with DCM. The dried residue afforded 26.61 mg (60.15% yield, 0.07 mmol) of the pure product.

¹H-NMR: (d6-DMSO, 300 MHz): 10.10 (s, 1H); 7.81 (sbr, 1H); 7.74 (sbr, 1H); 7.41-7.34 (m, 2H); 7.22 (dd, 1H); 6.56-6.48 (m, 2H); 4.71 (d, 1H); 4.56 (t, 1H); 4.20-4.07 (m, 2H); 4.14 (s, 1H); 3.60 (m, 1H); 3.29 (m, 2H); 1.95 (m, 1H); 1.65 (m, 1H).

MS (ESI): [M+H]⁺=377.

### Example Compound 9.2

### Preparation of 2-((R)-3,4-Dihydroxy-butoxy)-4-fluoro-6-[2-fluoro-4-(4-hydroxy-but-1-ynyl)-phenylamino]-benzamide

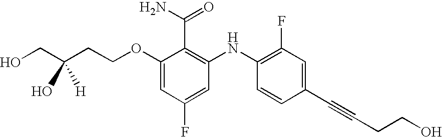

In analogy to GP 11b, 47.82 mg of 2-((R)-3,4-Dihydroxy-butoxy)-4-fluoro-6-(2-fluoro-4-iodo-phenylamino)-benzamide (0.1 mmol; 1 eq.) were dissolved in 0.5 ml of THF. Then 10.51 mg of but-3-yn-1-ol (0.15 mmol; 1.5 eq.) in 0.375 ml of THF was added, followed by 3.51 mg of dichlorobis(triphenylphosphine)palladium (II) (Pd(PPh₃)₂Cl₂) (0.005 mmol; 0.5 eq.) in 417 μl of THF and 130.73 mg of a 1M solution of tetra-N-butylammonium fluoride in THF (0.5 mmol; 5 eq.). The mixture was then allowed to react for 40 min at 110° C. in a microwave oven (600 W; max. 6 bar). The crude reaction mixture was directly submitted to preparative HPLC to yield 31.4 mg (74.69% yield; 0.075 mmol) of the pure target compound.

t_{R}=0.93 (HPLC conditions A); MW_{calc}=420.4; MW_{found}=421

The following example compounds 9.3 to 9.5 were prepared in analogy to the example above by Sonogashira coupling of the respective iodide substrates with TMS-acetylene or phenyl acetylene optionally followed by TMS deprotection.

| Example | Structure | Name | Analytical data |
|---|---|---|---|
| 9.3 | 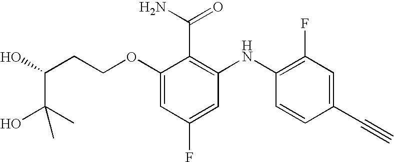 | 2-((R)-3,4-Dihydroxy-4-methyl-pentyloxy)-6-(4-ethynyl-2-fluoro-phenylamino)-4-fluoro-benzamide | ¹H-NMR: (d6-DMSO, 300 MHz):10.14 (s, 1 H); 7.78 (sbr, 1 H);7.77 (sbr, 1 H); 7.41-7.33(m, 2 H); 7.22 (dd, 1 H); 6.56-6.47(m, 2 H); 4.63 (d, 1 H);4.18-4.10 (m, 2 H); 4.16 (s, 1 H);4.14 (s, 1 H); 3.35-3.25(m, 1 H); 2.04 (m, 1 H); 1.58(m, 1 H); 1.05 (s, 3 H) 1.00 (s,3 H).MS (ESI): [M + H]⁺ = 405 |
| 9.4 | 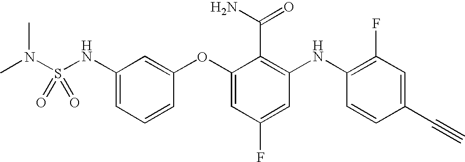 | 2-[3-[[(dimethylamino)sulfonyl]amino]phenyoxy]-4-fluoro-6-[(4-ethynyl-2-fluorophenyl)amino]-benzamide | ¹H-NMR (d₆-DMSO; 300 MHz):10.02 (s, 1 H); 9.16 (s, 1 H);7.89 (sbr, 1 H); 7.86 (sbr, 1 H);7.42-7.35 (m, 2 H); 7.32-7.21(m, 2 H); 6.95 (dd, 1 H);6.90 (dd, 1 H); 6.75-6.67(m, 2 H); 6.17 (dd, 1 H); 4.17(s, 1 H); 2.66 (s, 6H).MS (ESI): [M + H]⁺ = 487 |
| 9.5 | 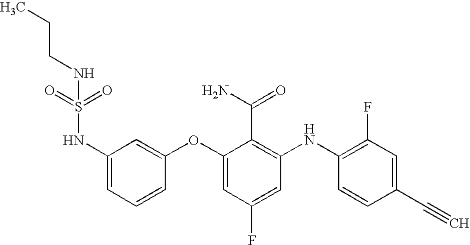 | 2-[3-[[(propylamino)sulfonyl]amino]phenyoxy]-4-fluoro-6-[(4-ethynyl-2-fluorophenyl)amino]-benzamide | MS (ESI): [M + H]⁺ = 501. |

### Example Compound 10.1

### Preparation of methanesulfonic acid (R)-4-[2-carbamoyl-5-fluoro-3-(2-fluoro-4-iodo-phenylamino)-phenoxy]-2-hydroxy-butyl ester

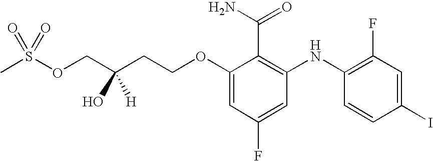

In analogy to GP 14, 1.1 g of 2-((R)-3,4-dihydroxy-butoxy)-4-fluoro-6-(2-fluoro-4-iodo-phenylamino)-benzamide (2.3 mmol, 1 eq.) were dissolved in 23 mL NMP and treated with 0.2 mL methansulfonyl chloride (2.53 mmol, 1.1 eq.) and 3.04 mL collidine (23 mmol, 10 eq.) at 0° C. and kept at this temperature overnight. Preparative HPLC purification of the crude reaction mixture provided the target compound.

MS (ESI): [M+H]⁺=557.

### Example Compound 10.2

### Preparation of 4-Fluoro-2-(2-fluoro-4-iodo-phenylamino)-6-[(R)-3-hydroxy-4-(2-hydroxy-ethylamino)-butoxy]-benzamide

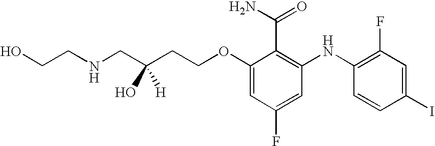

In analogy to GP 15, 1 eq. of methanesulfonic acid (R)-4-[2-carbamoyl-5-fluoro-3-(2-fluoro-4-iodo-phenylamino)-phenoxy]-2-hydroxy-butyl ester was dissolved in DMF (6 mL per 300 mg mesylate) and treated with 20 eq. hydroxyethylamine and stirred until final reaction turnover (by LCMS). Preparative HPLC purification provided the analytically pure target compound.

t_{R}=1.07 (HPLC conditions A); MW_{calc}=521.3; MW_{found}=522

The following example compounds 10.3 to 10.9 were prepared in analogy to Example compound 10.2 by applying other commercially available amine to the described reaction conditions.

| Example | Structure | Name | Analytical data |
|---|---|---|---|
| 10.3 | 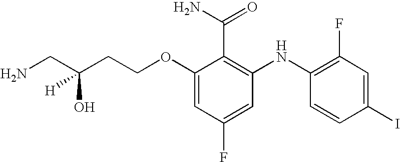 | 2-((R)-4-Amino-3-hydroxy-butoxy)-4-fluoro-6-(2-fluoro-4-iodo-phenylamino)-benzamide | t_{R} = 1.01(HPLC conditions A);MW_{calc} = 477.3;MW_{found} = 478 |
| 10.4 | 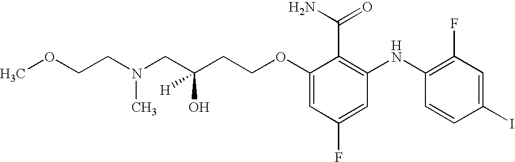 | 4-Fluoro-2-(2-fluoro-4-iodo-phenylamino)-6-{(R)-3-hydroxy-4-[(2-methoxy-ethyl)-methyl-amino]-butoxy}-benzamide | t_{R} = 1.11(HPLC conditions A);MW_{calc} = 549.3;MW_{found} = 550 |
| 10.5 | 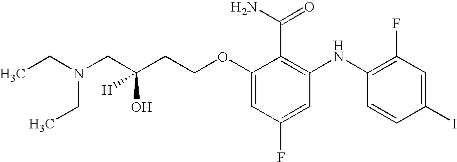 | 2-((R)-4-Diethylamino-3-hydroxy-butoxy)-4-fluoro-6-(2-fluoro-4-iodo-phenylamino)-benzamide | t_{R} = 1.13(HPLC conditions A);MW_{calc} = 533.3;MW_{found} = 534 |
| 10.6 | 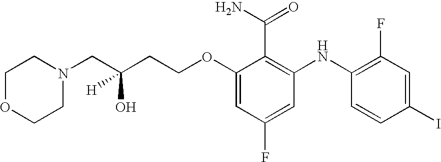 | 4-Fluoro-2-(2-fluoro-4-iodo-phenylamino)-6-((R)-3-hydroxy-4-morpholin-4-yl-butoxy)-benzamide | t_{R} = 1.09(HPLC conditions A);MW_{calc} = 547.3;MW_{found} = 548 |
| 10.7 | 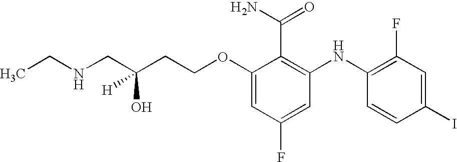 | 2-((R)-4-Ethylamino-3-hydroxy-butoxy)-4-fluoro-6-(2-fluoro-4-iodo-phenylamino)-benzamide | t_{R} = 1.11(HPLC conditions A);MW_{calc} = 505.3;MW_{found} = 506 |
| 10.8 | 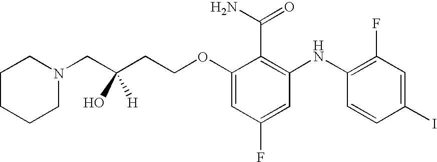 | 4-Fluoro-2-(2-fluoro-4-iodo-phenylamino)-6-((R)-3-hydroxy-4-piperidin-1-yl-butoxy)-benzamide | t_{R} = 1.10(HPLC conditions A);MW_{calc} = 545.4;MW_{found} = 546 |
| 10.9 | 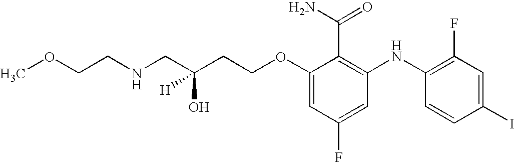 | 4-Fluoro-2-(2-fluoro-4-iodo-phenylamino)-6-[(R)-3-hydroxy-4-(2-methoxy-ethylamino)-butoxy]-benzamide | t_{R} = 1.11(HPLC conditions A);MW_{calc} = 535.3;MW_{found} = 536 |

The following example compounds 11.1 to 11.6 were synthesized by applying the afore described procedures starting from the respective 2,6-difluorobenzonitriles by stepwise substitution of the 6- and 2-fluoro substituent, subsequent nitrile hydrolysis and finally acetonide cleavage.

| Example | Structure | Name | Analytical data |
|---|---|---|---|
| 11.1 | 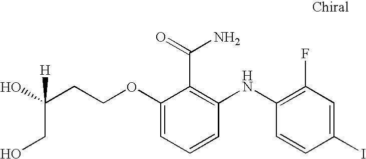 | 2-((R)-3,4-Dihydroxy-butoxy)-6-(2-fluoro-4-iodo-phenylamino)-benzamide | ¹H-NMR:(d6-DMSO, 300 MHz)9.41 (s, 1 H); 7.83 (br. s, 1 H);7.55 (br. s, 1 H); 7.62 (dd,1 H); 7.42 (d, 1 H); 7.26 (t, 1 H);7.17 (t, 1 H); 6.79 (d, 1 H);6.65 (d, 1 H); 4.72 (d, 1 H);4.60 (t, 1 H); 4.10-4.20(m, 2 H); 3.61-3.71 (m, 1 H);3.26-3.42 (m, 2 H); 1.92-2.05(m, 1 H); 1.63-1.76 (m,1 H). |
| 11.2 | 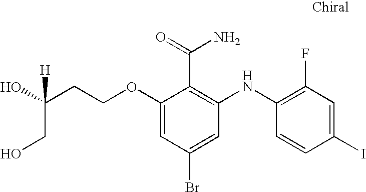 | 4-Bromo-2-((R)-3,4-dihydroxy-butoxy)-6-(2-fluoro-4-iodo-phenylamino)-benzamide | ¹H-NMR:(d6-DMSO, 300 MHz)9.51 (s, 1 H); 7.81 (br. s, 1 H);7.76 (br. s, 1 H); 7.63 (dd,1 H); 7.46 (d, 1 H); 7.13 (t, 1 H);6.70-6.78 (m, 2 H); 4.69(d, 1 H); 4.55 (t, 1 H); 4.07-4.18(m, 2 H); 3.55-3.65 (m,1 H); 3.21-3.37 (m, 2 H);1.87-1.98 (m, 1 H);1.58-1.70 (m, 1 H). |
| 11.3 | 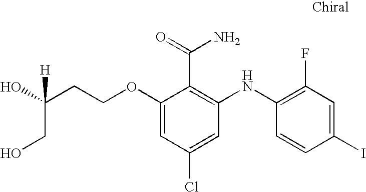 | 4-Chloro-2-((R)-3,4-dihydroxy-butoxy)-6-(2-fluoro-4-iodo-phenylamino)-benzamide | ¹H-NMR:(d6-DMSO, 300 MHz)9.65 (s, 1 H); 7.87 (br. s, 1 H);7.81 (br. s, 1 H); 7.68 (dd,1 H); 7.51 (d, 1 H); 7.20 (t, 1 H);6.61-6.70 (m, 2 H); 4.75(d, 1 H); 4.61 (t, 1 H); 4.13-4.25(m, 2 H); 3.60-3.69 (m,1 H); 3.26-3.41 (m, 2 H);1.92-2.04 (m, 1 H),1.61-1.75 (m, 1 H). |
| 11.4 | 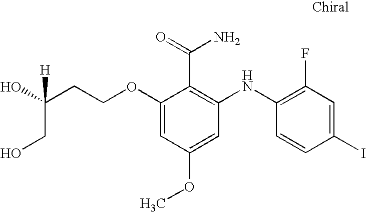 | 2-((R)-3,4-Dihydroxy-butoxy)-6-(2-fluoro-4-iodo-phenylamino)-4-methoxy-benzamide | ¹H-NMR:(d6-DMSO, 300 MHz)10.59 (s, 1 H); 7.81 (br. s, 1 H);7.65 (dd, 1 H); 7.56 (br. s,1 H); 7.47 (d, 1 H); 7.29 (t, 1 H);6.17 (d, 1 H); 6.30 (d, 1 H);4.76 (d, 1 H); 4.62 (t, 1 H);4.15-4.25 (m, 2 H); 3.74(s, 3 H); 3.60-3.68 (m, 1 H);3.25-3.41 (m, 2 H); 1.94-2.06(m, 1 H); 1.65-1.77 (m,1 H). |
| 11.5 | 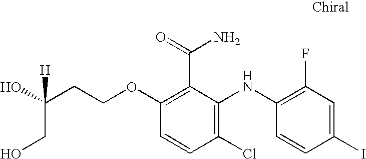 | 3-Chloro-6-((R)-3,4-dihydroxy-butoxy)-2-(2-fluoro-4-iodo-phenylamino)-benzamide | MS (ESI):[M + H]⁺ = 494/496 (Cl isotopepattern). |
| 11.6 | 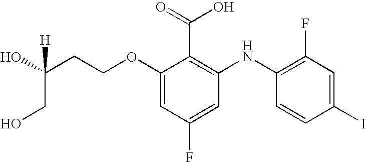 | 2-((R)-3,4-Dihydroxy-butoxy)-4-fluoro-6-(2-fluoro-4-iodo-phenylamino)-benzoic acid | MS (ESI): [M − H]− = 478 |

The following example compounds 12.1 to 12.14 were synthesized by standard transformations from the afore described example compounds, including i) Amide formation, ii) Suzuki coupling, epoxidation and subsequent nucleophilic epoxide opening, iv) alkylation, v) acetonide cleavage, yl) ester formation, vii) oxidative diol cleavage, and viii) protecting group cleavage.

| Example | Structure | Name | Analytical data |
|---|---|---|---|
| 12.1 | 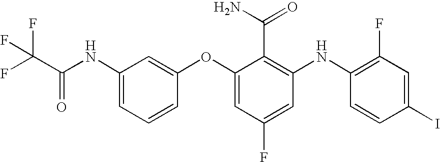 | 4-Fluoro-2-(2-fluoro-4-iodo-phenylamino)-6-[3-(2,2,2-trifluoro-acetylamino)-phenoxy]-benzamide | MS (ESI): [M + H]⁺ = 578 |
| 12.2 | 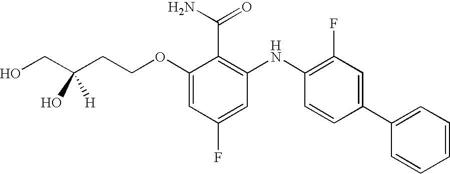 | 2-((R)-3,4-Dihydroxy-butoxy)-4-fluoro-6-(3-fluoro-biphenyl-4-ylamino)-benzamide | t_{R} = 1.28(HPLC conditions A);MW_{calc} = 428.4;MW_{found} = 429 |
| 12.3 | 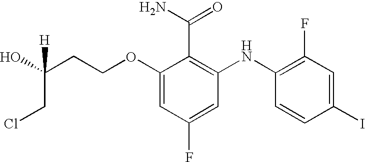 | 2-((R)-4-Chloro-3-hydroxy-butoxy)-4-fluoro-6-(2-fluoro-4-iodo-phenylamino)-benzamide | ¹H-NMR:(d6-DMSO, 600 MHz)9.86 (s, 1 H); 7.76-7.79 (m,2 H); 7.67 (dd, 1 H); 7.49 (d,1 H); 7.22 (t, 1 H); 6.51 (dd, 1 H);6.44 (dd, 1 H); 5.33 (d, 1 H);4.12-4.19 (m, 2 H);3.86-3.90 (m, 1 H); 3.61 (m, 2 H);2.01-2.07 (m, 1 H);1.79-1.84 (m, 1 H).MS (ESI):[M + H]⁺ = 497/499 (Cl isotopepattern). |
| 12.4 | 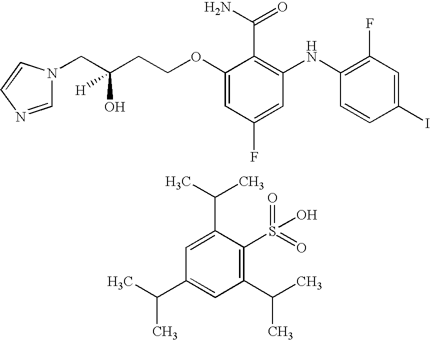 | 4-Fluoro-2-(2-fluoro-4-iodo-phenylamino)-6-((R)-3-hydroxy-4-imidazol-1-yl-butoxy)-benzamide;compound with2,4,6-triisopropyl-benzenesulfonic acid | [M + H]⁺ = 529. |
| 12.5 | 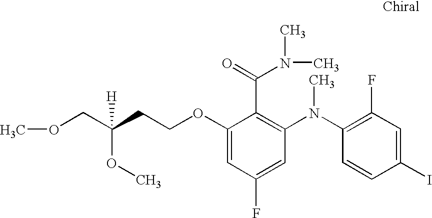 | 2-((R)-3,4-Dimethoxy-butoxy)-4-fluoro-6-[(2-fluoro-4-iodo-phenyl)-methyl-amino]-N,N-dimethylbenzamide | [M + H]⁺ = 549. |
| 12.6 | 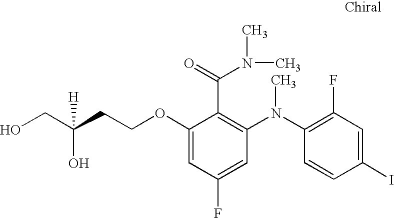 | 2-((R)-3,4-Dihydroxy-butoxy)-4-fluoro-6-[(2-fluoro-4-iodo-phenyl)-methyl-amino]-N,N-dimethyl-benzamide | ¹H-NMR:(d6-DMSO, 300 MHz)7.47 (dd, 1 H); 7.41 (dd, 1 H);6.82 (t, 1 H); 6.62 (ddd, 1 H);6.54 (dt, 1 H); 4.45-4.53 (m,2 H); 4.00 (t, 2 H); 3.44-3.52(m, 1 H); 3.16-3.30 (m, 2 H);3.10 (s, 3 H); 2.51 (d, 6 H);1.72-1.88 (m, 1 H);1.45-1.58 (m, 1 H). |
| 12.7 | 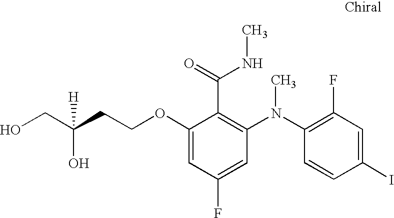 | 2-((R)-3,4-Dihydroxy-butoxy)-4-fluoro-6-[(2-fluoro-4-iodo-phenyl)-methyl-amino]-N-methyl-benzamide | [M + H]⁺ = 507. |
| 12.8 | 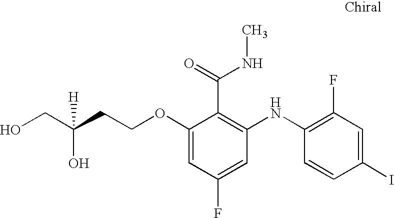 | 2-((R)-3,4-Dihydroxy-butoxy)-4-fluoro-6-(2-fluoro-4-iodo-phenylamino)-N-methyl-benzamide | ¹H-NMR:(d6-DMSO, 300 MHz)9.74 (s, 1 H); 8.37 (br. q, 1 H);7.63 (dd, 1 H); 7.44 (d, 1 H);7.17 (t, 1 H); 6.46 (dd, 1 H);6.39 (dd, 1 H); 4.77 (d, 1 H);4.59 (t, 1 H); 4.04-4.17(m, 2 H); 3.55-3.65 (m, 1 H);3.20-3.38 (m, 2 H); 2.72 (d,3 H); 1.88-1.98 (m, 1 H);1.60-1.72 (m, 1 H). |
| 12.9 | 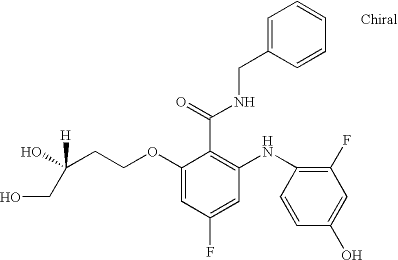 | N-Benzyl-2-((R)-3,4-dhydroxy-butoxy)-4-fluoro-6-(2-fluoro-4-iodo-phenylamino)-benzamide | [M + H]⁺ = 569. |
| 12.10 | 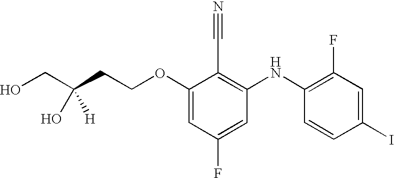 | 2-((R)-3,4-Dihydroxy-butoxy)-4-fluoro-6-(2-fluoro-4-iodo-phenylamino)-benzonitrile | MS (ESI): [M + H]⁺ = 461. |
| 12.11 | 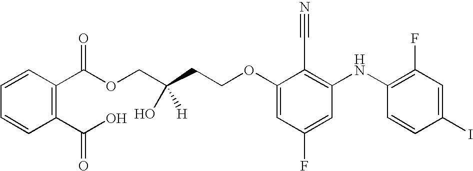 | Phthalic acid mono-{(R)-4-[2-cyano-5-fluoro-3-(2-fluoro-4-iodo-phenylamino)-phenoxy]-2-hydroxy-butyl}ester | MS (ESI): [M + H]⁺ = 609. |
| 12.12 | 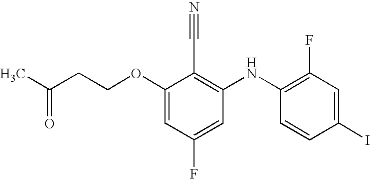 | 4-Fluoro-2-(2-fluoro-4-iodo-phenylamino)-6-(3-oxo-butoxy)-benzonitrile | MS (ESI): [M + H]⁺ = 443. |
| 12.13 | 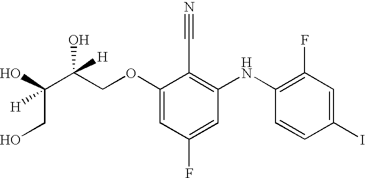 | 4-Fluoro-2-(2-fluoro-4-iodo-phenylamino)-6-((2R,3R)-2,3,4-trihydroxy-butoxy)-benzonitrile | MS (ESI): [M + H]⁺ = 477. |
| 12.14 | 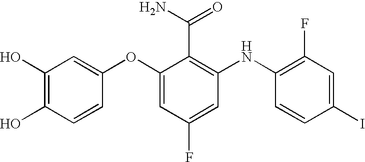 | 2-(3,4-Dihydroxy-phenoxy)-4-fluoro-6-(2-fluoro-4-iodo-phenylamino)-benzamide | MS (ESI): [M + H]⁺ = 499. |

### Example Compound 13.1

### Preparation of 2-[3-(3,3-Dimethyl-ureido)-phenoxy]-4-fluoro-6-(2-fluoro-4-iodo-phenylamino)-benzamide

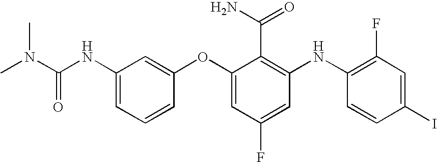

In analogy to GP 10, 45 mg of {2-Carbamoyl-3-[3-(3,3-dimethyl-ureido)-phenoxy]-5-fluoro-phenyl}-(2-fluoro-4-iodo-phenyl)-carbamic acid tert-butyl ester (0.071 mmol; 1 eq.) were dissolved in 2 ml DCM and 0.11 ml TFA (1.42 mmol; 20 eq.) were added. The mixture was stirred at RT for 12 h and then concentrated. The residue was partitioned between 10 ml of ethyl methyl ketone and 5 ml of 1M aq. sodium hydroxide solution. The aqueous layer was extracted twice with ethyl methyl ketone (10 ml each). The combined organic layers were washed with 10 ml half concentrated brine, dried via silicone filter and concentrated to afford 56.4 mg of the crude product. Purification was achieved by flash chromatography to afford 6.39 mg (16.31% yield; 0.012 mmol).

¹H-NMR: (d6-DMSO, 300 MHz) 9.17 (s, 1H); 8.37 (s, 1H); 7.84 (sbr, 1H); 7.81 (sbr, 1H); 7.66 (dd, 1H); 7.47 (dbr, 1H); 7.30-7.18 (m, 4H); 6.65 (dbr, 1H); 6.54 (dbr, 1H); 6.07 (dd, 1H); 2.87 (s, 6H).

MS (ESI): [M+H]⁺=553

Similarly, using appropriate starting materials and the experimental procedures described above, compounds in the following table may be prepared. It will be understood by those skilled in the art that some minor modifications to the described procedures may be necessary, but such modifications do not significantly affect the results of the preparation.

| | | Preparation |
|---|---|---|
| | | method |
| Example | | (Ref. Example |
| No. | Structure | No.) |
| 3.1 | 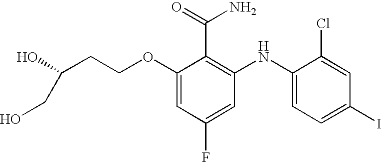 | 3 |
| 3.2 | 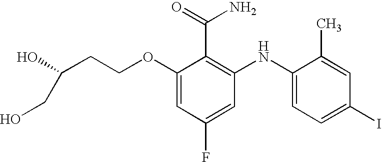 | 3 |
| 3.3 | 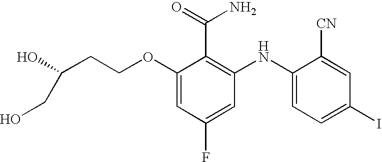 | 3 |
| 3.4 | 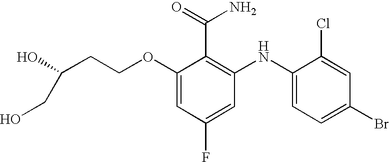 | 3 |
| 3.5 | 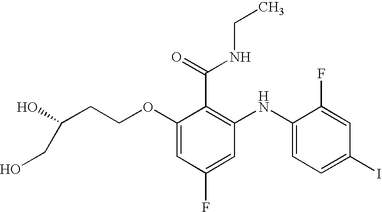 | 5 |
| 3.6 | 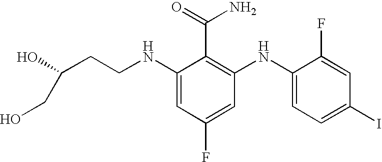 | 3 |
| 3.7 | 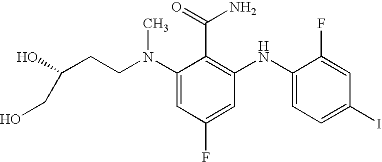 | 3 |
| 3.8 | 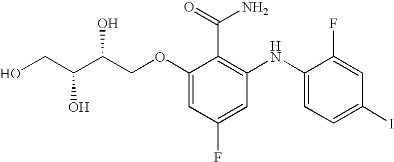 | 3 |

### Biological Evaluation

The utility of the compounds of the present invention can be illustrated, for example, by their activity in vitro in the in vitro tumor cell proliferation assay described below. The link between activity in tumor cell proliferation assays in vitro and anti-tumor activity in the clinical setting has been very well established in the art. For example, the therapeutic utility of taxot (Silvestrini et al. Stem Cells 1993, 11(6), 528-35), taxotere (Bissery et al., Anti Cancer Drugs 1995, 6(3), 339), and topoisomerase inhibitors (Edelman et al. Cancer Chemother. Pharmacol. 1996, 37(5), 385-93) were demonstrated with the use of in vitro tumor proliferation assays.

Demonstration of the activity of the compounds of the present invention may be accomplished through in vitro, ex vivo, and in vivo assays that are well known in the art. For example, to demonstrate the activity of the compounds of the present invention, the following assays may be used.

### Biological Assays

### Assay 1

### MEK Biochemical Assay: DELFIA

The DELFIA MEK kinase assay was used to monitor the activity of MEK inhibitors. The kinase reaction was carried out in a 96-well microtitration plate by firstly mixing 70 μL of kinase reaction buffer (50 mM HEPES pH 7.5, 5 mM NaF, 5 mM glycerophosphate, 1 mM sodium vanadate, 10 mM MgCl₂, 1 mM DTT and 1% (v/v) DMSO) with 20 nM GST-MEK, 20 nM His-Raf and 100 nM biotinylated ERK1 (final concentration). Then compounds with final concentrations of 1 μM, 0.3 μM, 0.1 μM, 0.03 μM, 0.01 μM, 0.003 μM, 0.001 μM, 0.0003 μM and 0 μM were added to generate the dose response inhibition curve. The kinase reaction was started by adding 20 μL of ATP (final concentration 100 μM). After 2 h incubation, the reaction was terminated by adding 20 μl of 0.5 M EDTA. Then 100 μL of the reaction mixture was transferred to a 96 well Streptavidin plate (cat #15120, Pierce Inc. Rockford, Ill.) and subsequently incubated for 2 h. After collecting the biotinylated substrate ERK1, the plate was washed with TBST. An antibody against phospho-p44/42 MAPK (cat# 91065, Cell Signaling Technologies, Danvers, Mass.) was added and bond to the phosphorylated substrate. Thereafter, incubation with an Europium-labeled anti-mouse antibody (cat# AD0124, Wallac Inc, Turku, Finland) followed by a washing step was carried out. The Enhancement Solution was added to dissociate europium ions into solution, where they formed highly fluorescent chelates with the components of the enhancement solution. The fluorescence of each sample was proportional to kinase activity and counted on a VICTOR5 instrument (Wallac Inc.). Data analysis was performed using Analyze5 software for IC₅₀ analysis. The following results were obtained for compounds tested:

IC₅₀ less than 0.4 μM: Examples 1, 4, 5, 10, 11, 12, and 13;
IC₅₀ between 0.4 μM and 1 μM: Examples 2, 6, and 8;
IC₅₀ between 1 μM and 2.5 μM: Examples 3, 7, and 9.

### Assay 2

### MEK1 Activation Kinase Assay

The kinase Cot1 activates MEK1 by phosphorylating its activation loop. The inhibitory activity of compounds of the present invention on this activation of MEK1 was quantified employing the HTRF assay described in the following paragraphs.

N-terminally His6-tagged recombinant kinase domain of the human Cot1 (amino acids 30-397, purchased from Millipore, cat. no 14-703) expressed in insect cells (SF21) and purified by Ni-NTA affinity chromatography was used as kinase. As substrate for the kinase reaction the unactive C-terminally His6-tagged GST-MEK1 fusion protein (Millipore cat. no 14-420) was used.

For the assay 50 nl of a 100 fold concentrated solution of the test compound in DMSO was pipetted into a black low volume 384 well microtiter plate (Greiner Bio-One, Frickenhausen, Germany), 3 μl of a solution of 24 nM GST-MEK1 and 166.7 μM adenosine-tri-phosphate (ATP) in assay buffer [50 mM Tris/HCl pH 7.5, 10 mM MgCl₂, 2 mM dithiothreitol, 0.01% (v/v) Igepal Calif. 630 (Sigma), 5 mM β-phospho-glycerol] were added and the mixture was incubated for 10 min at 22° C. to allow pre-binding of the test compounds to the GST-MEK1 before the start of the kinase reaction. Then the kinase reaction was started by the addition of 2 μl of a solution of Cot1 in assay buffer and the resulting mixture was incubated for a reaction time of 20 min at 22° C. The concentration of Cot1 in the assay was adjusted depending of the activity of the enzyme lot and was chosen appropriate to have the assay in the linear range, typical enzyme concentrations were in the range of about 2 ng/μl (final conc. in the 5 μl assay volume). The reaction was stopped by the addition of 5 μl of a solution of HTRF detection reagents (13 nM anti GST-XL665 [# 61GSTXLB, Fa. Cis Biointernational, Marcoule, France], 1 nM Eu-cryptate labelled anti-phospho-MEK 1/2 (Ser217/221) [#61P17KAZ, Fa. Cis Biointernational],) in an aqueous EDTA-solution (100 mM EDTA, 500 mM KF, 0.2% (w/v) bovine serum albumin in 100 mM HEPES/NaOH pH 7.5). The resulting mixture was incubated 2 h at 22° C. to allow the binding of the phosphorylated GST-MEK1 to the anti-GST-XL665 and the Eu-cryptate labelled anti-phospho-MEK 1/2 antibody. Subsequently the amount of Ser217/Ser221-phosphorylated substrate was evaluated by measurement of the resonance energy transfer from the Eu-Cryptate-labelled anti-phospho-MEK antibody to the anti-GST-XL665. Therefore, the fluorescence emissions at 620 nm and 665 nm after excitation at 350 nm was measured in a HTRF reader, e.g. a Rubystar (BMG Labtechnologies, Offenburg, Germany) or a Viewlux (Perkin-Elmer). The ratio of the emissions at 665 nm and at −622 nm was taken as the measure for the amount of phosphorylated substrate. The data were normalised (enzyme reaction without inhibitor=0% inhibition, all other assay components but no enzyme=100% inhibition). Normally test compound were tested on the same microtiter plate at 10 different concentrations in the range of 20 μM to 1 nM (20 μM, 6.7 μM, 2.2 μM, 0.74 μM, 0.25 μM, 82 nM, 27 nM, 9.2 nM, 3.1 nM and 1 nM, dilution series prepared before the assay at the level of the 100 fold conc. stock solutions by serial 1:3 dilutions) in duplicate values for each concentration and IC₅₀ values were calculated by a 4 parameter fit using an inhouse software.

The following representative example compounds show an IC₅₀ below 1 μM in this assay: Examples 2.1, 3.2, 3.3, 3.5, 3.8, 4.1, 4.5, 4.6, 5.1, 5.2, 6.1a, 6.3, 6.6, 6.7, 6.11, 6.15, 6.17, 6.22, 7.1, 7.7, 8.4, 8.5, 8.6, 9.1, 9.4, 9.5, 10.3, 10.6, 11.3, 12.8.

The following representative example compounds show an IC₅₀ below 250 nM: Examples 3.2, 3.3, 3.5, 3.8, 4.1, 4.5, 4.6 5.2, 6.1a, 6.3, 6.6, 6.7, 6.11, 6.17, 7.1, 7.7, 8.6, 9.4, 9.5, 10.3, 10.6, 12.8.

### Assay 3

### Phospho-ERK Mechanistic Assay

A375 and Colo205 cells were plated in RPMI 1640 growth medium supplemented with 10% FBS at 25,000 cells per well in 96-well tissue culture plates. Cells were incubated overnight in a humidified incubator containing 5% CO₂ at 37° C. The following day, to prepare the assay plates, anti-rabbit Meso-Scale Discovery (MSD) plates (cat# L41 RA-1, Meso-Scale Discovery, Gaithersburg, Md.) were blocked with 100 μl of 5% MSD blocking buffer for 1 h at room temperature, after which they were washed three times with 200 μl of TBST buffer. The phospho-ERK rabbit polyclonal antibody (cat# 9101, Cell Signaling Technologies, Danvers, Mass.) diluted at 1:200 into 2.5% of MSD Blocker A-TBST was added (25 μl) to each well and the plate was then incubated 1 h at room temperature with shaking. The plates were then washed once with phosphate buffered saline (PBS) and ready to receive the cell lysates. While the preparation of the assay plates was ongoing, test compounds were added to the wells of cell-containing plates from the previous day, serially diluted in RPMI 1640 medium containing 10% FBS, 0.1% bovine serum albumin (BSA) and 0.03% DMSO and the plates were incubated for 1.5 h at 37° C. After this incubation, the compound-treated plates were washed three times with PBS, lysed in 30 μl of Bio-Rad lysis buffer (cat #98601, Bio-Rad Laboratories, Hercules, Calif.) and then left shaking on ice for 30 min. The lysates were then loaded on the phospho-ERK coated MSD plates and the plates Incubated overnight at 4° C. The following day, the plates were washed three times with TBST and 25 μl of 1:3000 diluted total ERK monoclonal antibody (Cat# 610123, BD Biosciences, San Diego, Calif.) was added to the plates that were then incubated 1 h at room temperature with shaking. After the incubation the plates were washed three times with TBST as described earlier and 25 μl of MSD sulfo-tag anti-mouse antibody (cat #R32AC-5) diluted 1:1000 were added into each well. The plates were Incubated 1 h at room temperature with shaking, then washed four times with TBST. Just prior to reading the plates, 150 μl of MSD Read buffer T was added and the plates were read immediately on the MSD instrument. Data analysis was performed using Analyze5 software for IC₅₀ analysis. All compounds tested had an IC₅₀ below 3 μM.

### Assay 4

### Alternative Conditions for Mechanistic Perk Assay

For the measurement of ERK1/2 phosphorylation in tumor cell Lines a singleplex Mesoscale Discovery (MSD) assay is used. This assay is built up Like a sandwich immunoassay. Cell Lysates generated from different tumor cell lines treated with serially diluted MEK inhibitor compounds were Loaded on the MSD plates. Phosphorylated ERK1/2 present in the samples binds to the capture antibody immobilized on the working electrode surface. The sandwich is completed by binding of a detection antibody to the immobilized phospho-ERK1/2. This detection antibody is labeled with an electro-chemiluminescent compound. Applying voltage to the plate electrodes causes the labels, bound to the electrode surface via the antibody-phospho ERK1/2 sandwich complex, to emit light. The measurement of the emitted light allows a quantitative determination of the amount of phosphorylated ERK1/2 present in the sample. In detail, a linear range for the measurement of phosphoERK signals must be determined for every cell line used in the assay by titrating different cell numbers. For the final assay, the previously determined cell number is seeded in 96 well plates. 24 h after seeding, cells were treated for 1.5 h with serially diluted allosteric MEK inhibitor compounds before the cells were Lysed and Lysates were transferred in the MSD assay plate. The manufacturer's protocol was changed in that the binding step of the phosphorylated ERK to the capture antibody was performed over night at 4° C. instead of 3 h at room temperature, leading to a better signal strength.

A375 or Colo205 cells were plated in 50 μL DMEM growth medium (Biochrom FG 0435) supplemented with 10% FBS (Biochrom #S0410) (A375), respectively in RPMI growth medium (Biochrom FG1215) supplemented with 10% FBS (Biochrom #S0410), 10 mM HEPES (Biochrom L1613), 4.5 g/L Glucose and 1 mM sodiumpyruvat (Biochrom L0473) (Colo-205) at 45000 cells per well in 96-well tissue culture plates. Cells were incubated overnight in a humidified incubator containing 5% CO₂ at 37° C.

The Phospho-ERK by Mesoscale Discovery (MSD) (# K111DWD) assay was performed according to the manufacturer's recommendations. In brief the protocol was:

The day after cell seeding, to prepare the assay plates, MSD were blocked with 150 μl of MSD blocking buffer for 1 h at room temperature, after which they were washed four times with 150 μl of Tris Wash buffer. While the preparation of the assay plates was ongoing, test compounds were added to the wells of cell-containing plates from the previous day, serially diluted in respective growth medium containing 10% FBS and 0.1% DMSO and the plates were incubated for 1.5-2 h at 37° C. After this incubation the medium was aspirated, cells were lysed in 50 μl lysis buffer and then left shaking for 30 min at 4° C. 25 μL of the lysates were then loaded on the blocked MSD plates and the plates Incubated overnight at 4° C. The following day, the plates were washed four times with Tris wash buffer and 25 μl detection antibody solution was added to the plates that were then incubated 1 h at room temperature with shaking. After the incubation the plates were washed four times with Tris wash buffer 150 μl of MSD Read buffer T was added and the plates were read immediately on the MSD instrument. Data analysis was performed using an in-house software for IC₅₀ analysis. All compounds tested had an IC₅₀ below 3 μM.

### Assay 5

### In Vitro Tumor Cell Proliferation Assay:

The adherent tumor cell proliferation assay used to test the compounds of the present invention involves a readout called Cell Titre-Glo developed by Promega (Cunningham, BA “A Growing Issue: Cell Proliferation Assays. Modern kits ease quantification of cell growth” The Scientist 2001, 15(13), 26, and Crouch, S P et al., “The use of ATP bioluminescence as a measure of cell proliferation and cytotoxicity” Journal of Immunological Methods 1993, 160, 81-88).

A375 and Colo205 cells were plated in RPMI 1640 growth medium supplemented with 10% FBS at 3,000 cells per well in 96-well tissue culture plates. Cells were incubated overnight in a humidified incubator containing 5% CO₂ at 37° C. The following day, test compounds were added to wells, serially diluted in RPMI 1640 medium containing 10% FBS and 0.03% DMSO and the plates were incubated for 72 h at 37° C. Evaluation of cell density was made at different time points (0 and 72 h post-dosing) by adding to each well 150 μl of Cell Titer Glo reagent (cat# G7572, Promega, Madison Wis.) followed by incubation of the plates on a rotator for 10 min at room temperature and then reading of the luminescence on a Victor3 instrument. Data analysis was performed using Analyze5 software for IC₅₀ analysis. All compounds showed responses at concentrations below 10 μM.

### Assay 6

### In Vitro Tumor Cell Proliferation Assay in A375 Cells (Cell Titer Show ICTG1 Assay)

A375 cells [human malignant melanoma cells, ATCC # CRL-1619, expressing mutant BRAF V600E] were plated at a density of 3000 cells/well in 96 well black-clear bottom tissue culture plates (Costar 3603 black/clear bottom) in 100 μL/well DMEM medium (Biochrom; FG0435; +3.7 g/L odium bicarbonate; +4.5 g/L D-Glucose) with 10% Fetal Bovine Serum (FBS) and stable Glutaminincubated at 37° C. Plate sister wells in separate plate for time zero determination. Incubate all plates overnight 37° C. Take down time zero plate: add 67 μL/well CTG solution (Promega Cell Titer Glo solution) to time zero wells in sister plate; the plates were mixed for 2 min on orbital shaker to ensure cell lysis, incubate 10 minutes, read luminescence on VICTOR3 (Perkin Elmer). Twenty-four hours after cell seeding, test compounds diluted in 50 μL medium are added at a final concentration range from as high 10 μM to as low 300 pM depending on the activities of the tested compounds in serial dilutions at a final DMSO concentration of 0.4%. Cells were incubated for 72 hours at 37° C. after addition of the test compound. Then, using a Promega Cell Titer Glo Luminescent® assay kit, 100 microliters lysis buffer containing of the enzyme luciferase and its substrate, luciferin mixture, were added to each well and incubated for 10 min at room temperature in the dark to stabilize luminescence signal. The samples were read on VICTOR3 (Perkin Elmer) using Luminescence protocol. The percentage change in cell growth was calculated by normalizing the measurements to the extinctions of the zero point plate (=0%) and the extinction of the untreated (0 μM) cells (=100%). The IC50 values were determined by means of a 4-parameter fit using the company's own software.

Alternatively, the Cell Proliferation was Measured by Crystal Violet (CV) Staining: Assay 7

Cultivated human A375 cells were plated out in a density of 1500 cells/measurement point in 200 μl of growth medium (DMEM/HAMS F12 (Biochrom; FG4815) with 10% FBS and 2 mM Glutamine) in a 96-well multititer plate. After 24 hours, the cells from a plate (zero plate) were stained with crystal violet (see below), while the medium in the other plates was replaced by fresh culture medium (200 μl) to which the test substances had been added in various concentrations (0 μM, and in the range 0.3 nM-30 μM; the final concentration of the solvent dimethyl sulphoxide was 0.5%). The cells were incubated in the presence of the test substances for 4 days. The cell proliferation was determined by staining the cells with crystal violet: the cells were fixed by adding 20 μl/measurement point of an 11% glutaraldehyde solution at room temperature for 15 min. After the fixed cells had been washed three times with water, the plates were dried at room temperature. The cells were stained by adding 100 μl/measurement point of a 0.1% crystal violet solution (pH adjusted to pH 3 by adding acetic acid). After the stained cells had been washed three times with water, the plates were dried at room temperature. The dye was dissolved by adding 100 μl/measurement point of a 10% acetic acid solution, and the extinction was determined by photometry at a wavelength of 595 nm. The percentage change in cell growth was calculated by normalizing the measurements to the extinctions of the zero point plate (=0%) and the extinction of the untreated (0 μM) cells (=100%). The IC₅₀ values were determined by means of a 4-parameter fit using the company's own software.

In vitro inhibition of proliferation of further cancer cell lines can be measured in analogy to the afore-described procedures. Details for exemplary further tumor cells lines are given below:

| | Indication | Ras or | | cell | |
|---|---|---|---|---|---|
| | (all | Raf | | number | |
| Cells | human) | Mutation | Method | per well | medium |
| A-431 | epidermoid | | CTG | 3000 | DMEM/HAMS F12 |
| | cancer | | | | (Biochrom; FG4815) + 10% |
| | | | | | FBS and stable Glutamin |
| A-431 | epidermoid | | CTG | 3000 | DMEM/HAMS F12 |
| non- | cancer | | | | (Biochrom; FG4815) + 10% |
| adherent | | | | | FBS and stable Glutamin |
| | | | | | (Plates were coated |
| | | | | | with poly-2-hydroxy- |
| | | | | | ethylmethacrylate before |
| | | | | | cell seeding) |
| A549 | lung | KRAS | CTG | 2000 | DMEM/HAMS F12 |
| | carcinoma | G12S | | | (Biochrom; FG4815) + 10% |
| | | | | | FBS and stable Glutamin |
| Colo-205 | colon | BRAF | CTG | 3000 | RPMI1640 (Biochrom; |
| | carcinoma | V600E | | | FG1215) + 10% heat |
| | | | | | inactivated FBS and stable |
| | | | | | glutamin + 1x non- |
| | | | | | essentiell amino acid + |
| | | | | | 1 mM Sodiumpyruvat + |
| | | | | | 10 mM Hepes |
| HCT-116 | colon | KRAS | CTG | 3000 | DMEM/HAMS F12 |
| | cancer, | G13D | | | (Biochrom; FG4815) + 10% |
| | | | | | FBS and stable Glutamin |
| HT-29 | colon | BRAF | CTG | 2000 | DMEM/HAMS F12 |
| | cancer | V600E | | | (Biochrom; FG4815) + 10% |
| | | | | | FBS and stable Glutamin |
| Lox | melanoma | BRAF | CTG | 2000 | RPMI1640 (Biochrom; |
| | | V600E | | | FG1215) + 10% heat |
| | | | | | inactivated FBS and |
| | | | | | stable glutamin + 1x non- |
| | | | | | essentiell amino acid + |
| | | | | | 1 mM Sodiumpyruvat |
| MCF-7 | breast | | CTG | 5000 | RPMI1640 (F1275; w/o |
| | cancer | | | | phenol red) + 10% FBS + |
| | | | | | 2 mM Glutamin + 2 mU/mL |
| | | | | | Insulin + 1E−10M estradiol |

### Assay 8

### In Vivo Efficacy Studies: Staged Human Xenograft Models

The in vivo anti-tumor activity of lead compounds was assessed in mice using xenograft models of human BRAF mutant melanoma and colon carcinomas. The Female athymic NCR nude mice were implanted subcutaneously with either a human melanoma (LOX), or a human colon (Colo205) carcinoma lines acquired from American Type Culture Collection (ATCC, Maryland). Treatment was initiated when tumors reached approximately 100 mg in size. Compounds were administered orally and freshly prepared in PEG/water (80%/20% respectively). The general health of mice was monitored and mortality was recorded daily. Tumor dimensions and body weights were recorded twice a week starting with the first day of treatment. Animals were euthanized according to Bayer IACUC guidelines. Treatments producing greater than 20% lethality and/or 20% net body weight loss were considered ‘toxic’.

Tumor growth was measured with electronic calipers three times a week and tumor weight (mg) calculated according to the following formula: [length (mm)×width (mm)²]/2. Anti-tumor efficacy was determined as a function of tumor growth inhibition (% TGI). TGI is calculated on days of measurement using the following formula: (100−mean tumor value of treated (T)/mean tumor of control value (C)×100)=% T/C. The control used in the calculations is either the “untreated control” or “vehicle”, whichever provides the most conservative representation of the data. A compound demonstrating a TGI of greater than or equal to 50% is considered active. Statistical significance is determined using either a one-tailed or two-tailed Student's T-Test. The compounds that were tested showed significant dose-dependent tumor growth inhibition in both LOX and Colo205 models.

Compounds of the invention were tested for activity using one or more of the assay procedures presented above.

It is believed that one skilled in the art, using the preceding information and information available in the art, can utilize the present invention to its fullest extent. Those skilled in the art will recognize that the invention may be practiced with variations on the disclosed structures, materials, compositions and methods without departing from the spirit or scope of the invention as it is set forth herein and such variations are regarded as within the ambit of the invention. The compounds described in the examples are intended to be representative of the invention, and it will be understood that the scope of the invention is not limited by the scope of the examples. The topic headings set forth above are meant as guidance where certain information can be found in the application, but are not intended to be the only source in the application where information on such topics can be found. All publications and patents cited above are incorporated herein by reference.

### REFERENCES

[1] American Cancer Society, Cancer Facts and Figures 2005.
[2] Sausville E A, El Sayed Y, Monga M, Kim G. Signal Transduction Directed Cancer Treatments. Annu Rev Pharmacol Toxicol 2002; 43: 199-231.
[3] O'Dwyer M E, Mauro M J, Druker B J. ST1571 as a targeted therapy for CML. Cancer Invest 2003; 21: 429-438.
[4] de Jong F A, Verweij J. Role of imatinib mesylate (Gleevec/Glivec) in gastrointestinal stromal tumors. Expert Rev Anticancer Ther 2003; 3: 757-766.
[4] Becker J. Signal transduction inhibitors—a work in progress. Nature Biotech 2004; 22: 15-18.
[5] Cobb M H. MAP kinase pathways. Prog Biophys Mol Biol 1999; 71: 479-500.
[6] Lewis T S, Shapiro P S, Ahn N G. Signal transduction through MAP kinase cascades. Adv Cancer Res 1998; 74: 49-139.
[7] English J M, Cobb M H. Pharmacological inhibitors of MAPK pathways. Trends Pharmacol Sci 2002; 23: 40-45.
[8] Duesbery N S, Webb C P, Vande Woude G F. MEK wars, a new front in the battle against cancer. Nat Med 1999; 5: 736-737.
[9] Sebolt-Leopold J S. Development of anticancer drugs targeting the MAP kinase pathway. Oncogene 2000; 19: 6594-6599.
[10] Milella M, Precupanu C M, Gregorj C, Ricciardi M R, Petrucci M T, Kornblau S M, Tafuri A, Andreeff M. Beyond single pathway inhibition: MEK inhibitors as a platform for the development of pharmacological combinations with synergistic anti-leukemic effects. Curr Pharm Des. 2005; 11 (21):2779-95.
[11] Hancock C N, Macis A T, Mackerell A D Jr, Shapiro P. Mitogen activated protein (MAP) kinases: development of ATP and non-ATP dependent inhibitors. Med. Chem. 2006 March;2(2):213-22.
[12] Deramaudt T, Rustgi A K. Mutant KRAS in the initiation of pancreatic cancer. Biochim Biophys Acta. 2005; 1756(2):97-101.
[13] Libra M, Malaponte G, Navolanic P M, Gangemi P, Bevelacqua V, Proietti L, Bruni B, Stivala F, Mazzarino M C, Travali S, McCubrey J A. Analysis of BRAF mutation in primary and metastatic melanoma. Cell Cycle. 2005 October;4(10):1382-4.
[14] Herrera R, Sebolt-Leopold J S. Unraveling the complexities of the Raf/MAP kinase pathway for pharmacological intervention. Trends Mol Med 2002; 8: S27-S31.
[15] Alessi D R, Cuenda A, Cohen P, Dudley D T, Saltiel A R. PD 098059 is a specific inhibitor of the activation of mitogenactivated protein kinase kinase in vitro and in vivo. J Biol Chem 1995; 270: 27489-27494.
[16] Favata M F, Horiuchi K Y, Manos E J, Daulerio A J, Stradley D A, Feeser W S, et al. Identification of a novel inhibitor of mitogenactivated protein kinase kinase. J Biol Chem 1998; 273: 18623-18632.
[17] Allen L F, Sebolt-Leopold J, Meyer M B. CI-1040 (PD184352), a targeted signal transduction inhibitor of MEK (MAPKK). Semin Oncol 2003; 30: 105-116.
[18] Sebolt-Leopold J S, Dudley D T, Herrera R, Van Becelaere K, Wiland A, Gowan R C, et al. Blockade of the MAP kinase pathway suppresses growth of colon tumors in vivo. Nat Med 1999; 5: 810-816
[19] Waterhouse D, Rinehart J, Adjei A, Hecht J, Natale R, LoRusso P, et al. A phase 2 study of an oral MEK inhibitor, CI-1040, in patients with advanced non small-cell lung, breast, colon, or pancreatic cancer. Proc Am Soc Clin Oncol 2003; 22: 204a (abstr).

1. A compound of general formula (I): 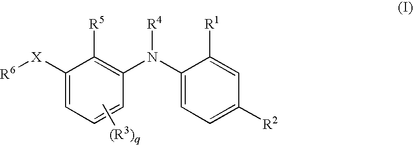
in which:
R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group q is an integer of 0, 1, 2, or 3;
R⁴ is a hydrogen atom or a C₁-C₆-alkyl group
R⁵ is a —C(═O)R⁷, —C(═O)OR⁷, —C(═O)N(R⁷)(R⁸), —NHC(═O)R⁷, —S(═O)₂R⁷, —NHS(═O)₂R⁷, —S(═O)₂NR⁷R⁸, —NO₂, —CN, or a 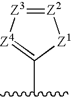
group,
in which
each of Z¹, Z², Z³ and Z⁴ is independently —CH—, —C(C₁-C₆-alkyl)-, —C(═O)—, —S—, —O—, —N— or —NH, such that at least one of Z¹, Z², Z³ and Z⁴ is —N— or —NH—;
X is —O—, —NH—, —N(C₁-C₆-alkyl)-, —S—, —S(═O)₂—, —C(═O)—, —C(═O)O—, —C(═O)NH—, or —NHC(═O)—;
R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R⁹, —(CH₂)ₙ—(CHN((R¹²)(R¹³)))—(CH₂)ₘ—R^{o}, —(CR¹⁵₂)ₙ—(CR¹⁵N((R¹²)(R¹³)))—(CR¹⁵₂)ₘ—R¹⁰, —(CH₂)ₙ—Y, —(CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), aryl, heteroaryl, C₂-C₁₀-alkenyl, C₅-C₁₀-cycloalkenyl, cycloalkyl or heterocycloalkyl group, in which aryl, heteroaryl, cycloalkyl, or heterocycloalkyl is optionally substituted with one or more —(CH₂)ₒR¹⁴ groups;
R⁷ and R⁸ are independently a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O— (CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹, R¹² and R¹³ are independently a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is, independently, a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NHS(═O)₂H, —NR^{a}S(═O)₂R^{b}, —S(═O)₂R^{b} or —C(═O)R^{b} group;
each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2;
each occurrence of R^{a} is, independently, a hydrogen atom or a C₁-C₆-alkyl group;
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group;
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-, 6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that:
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

2. The compound according to claim 1, wherein
R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group q is an integer of 0, 1, 2, or 3;
R⁴ is a hydrogen atom or a C₁-C₆-alkyl group;
R⁵ is a —C(═O)R⁷
R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R⁹, —(CH₂)ₙ—(CHN((R¹²)(R¹³)))—(CH₂)ₘ—R¹, —(CR¹⁵₂)ₙ—(CR¹⁵N((R¹²)(R¹³)))—(CR¹⁵₂)ₘ—R¹⁰, —(CH₂)ₙ—Y, —(CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), aryl, heteroaryl, C₂-C₁₀-alkenyl, C₅-C₁₀-cycloalkenyl, cycloalkyl or heterocycloalkyl group, in which aryl, heteroaryl, cycloalkyl, or heterocycloalkyl is optionally substituted with one or more —(CH₂)ₒR¹⁴ groups;
R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group, in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NR^{a}S(═O)₂R^{b}, —S(═O)₂R^{b} or —C(═O)R^{b} group;
each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2;
each occurrence of R^{a} is, independently, a hydrogen atom or a C₁-C₆-alkyl group;
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group;
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2} C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-, 6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that:
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

3. The compound according to claim 1, wherein
R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group q is an integer of 0, 1, 2, or 3;
R⁴ is a hydrogen atom or a C₁-C₆-alkyl group;
R⁵ is a —C(═O)R⁷
R⁶ is —(CH₂)ₙ—(CH(OR¹¹))—(CH₂)ₘ—R⁹, —(CR¹⁵₂)ₙ—(CR¹⁵(OR¹¹))—(CR¹⁵₂)ₘ—R⁹, —(CH₂)ₙ—(CHN((R¹²) (R¹³)))—(CH₂)ₘ—R¹⁰, —(CR¹⁵₂)ₙ—(CR¹⁵N((R¹²)(R¹³)))—(CR¹⁵₂)ₘ—R¹⁰, —(CH₂)ₙ—Y, (CH₂)ₙ—CH(OH)—CH(OH)—CH₂(OH), or —(CH₂)ₙ—CH(OH)—C(═O)OH;
Y is —S(═O)₂NH₂, —S(═O)₂NH(C₁-C₃-alkyl), —N(R¹²)(R¹³), C₂-C₁₀-alkenyl, C₅-C₁₀-cycloalkenyl, cycloalkyl or heterocycloalkyl group, in which cycloalkyl or heterocycloalkyl is optionally substituted with one or more —(CH₂)ₒR¹⁴ groups R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group; in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is a halogen atom, C₁-C₆ alkoxy, C₁-C₆ alkylamino or (C₁-C₆-alkyl)₂-amino;
each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2;
each occurrence of R^{a} is, independently, a hydrogen atom or a C₁-C₆-alkyl group;
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group;
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-, 6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

4. The compound according to claim 1, wherein
R¹ and R² are the same or different and are independently a hydrogen atom, a halogen atom, a C₁-C₆-alkyl, C₂-C₆-alkenyl, C₂-C₆-alkynyl, or —CN group, in which at least one of R¹ and R² is a halogen atom;
each occurrence of R³ is independently a halogen atom, a C₁-C₄-alkyl or —CN group;
q is an integer of 0, 1, 2, or 3;
R⁴ is a hydrogen atom or a C₁-C₆-alkyl group;
R⁵ is a —C(═O)R⁷
R⁶ is —(CH₂)ₙ—Y;
Y is aryl, heteroaryl, in which aryl, heteroaryl is optionally substituted with one or more —(CH₂)ₒR¹⁴ groups;
R⁷ is a —N(R¹²)(R¹³), —OH, or a —C₁-C₆-alkoxy group;
R⁸ is a hydrogen atom, a —N(R¹²)(R¹³), —OH, —C₁-C₆-alkoxy, —C₁-C₆-alkyl, —CF₃, —O—(CH₂)ₙ—(CH(OR¹¹))—(CH₂)ₘ—R⁹, —O—(CH₂)ₙ-cycloalkyl, aryl, heteroaryl, cycloalkyl or heterocycloalkyl group, in which aryl, heteroaryl, cycloalkyl, or heterocycloalkyl are, independently of each other, optionally substituted with one or more halogen atoms, C₁-C₆-alkyl or C₁-C₆-alkoxy groups;
R⁹ and R¹⁰ are independently —OH, —C₁-C₆-alkoxy, halogen, heteroaryl, —NR^{d1}R^{d2} or —N(R¹²)(R¹³);
R¹¹ is a hydrogen atom, a C₁-C₆-alkyl, aryl, heteroaryl, cycloalkyl or heterocycloalkyl group, in which C₁-C₆-alkyl, aryl, heteroaryl, cycloalkyl, or heterocycloalkyl are, independently of each other, optionally substituted with one or more —(CH₂)ₒR¹⁴ groups,
R¹² and R¹³ are independently a hydrogen atom or a C₁-C₆-alkyl group, in which C₁-C₆-alkyl is optionally substituted with one R¹⁴ group;
or
R¹² and R¹³, together with the N atom to which they are bound, form a 5-, 6-, or 7-membered heterocyclic ring which optionally comprises one or more additional heteroatoms, which optionally comprises one or more —C(═O)— or —S(═O)₂ groups, and which is optionally substituted with one or more —(CH₂)ₒR¹⁴ groups;
each occurrence of R¹⁴ is a halogen atom, a C₁-C₆-alkyl, C₁-C₆-haloalkyl, C₁-C₆-alkoxyalkyl, cycloalkyl, heterocycloalkyl, —OR^{c}, —NR^{d1}R^{d2}, —CN, —NR^{a}S(═O)₂R^{b}, —S(═O)₂R^{b} or —C(═O)R^{b} group;
a halogen atom, C₁-C₆ alkoxy, C₁-C₆ alkylamino or (C₁-C₆-alkyl)₂-amino each occurrence of R¹⁵ is, independently, a hydrogen atom or a C₁-C₆-alkyl group;
each occurrence of n is, independently, an integer of 0, 1, 2, 3, or 4;
each occurrence of m is, independently, an integer of 0, 1, or 2; and
each occurrence of o is, independently, an integer of 0, 1, or 2;
each occurrence of R^{a} is, independently, a hydrogen atom or a C₁-C₆-alkyl group;
each occurrence of R^{b} is, independently, an —OH, —OR^{c}, —SR^{c}, —NR^{d1}R^{d2}, a C₁-C₆-alkyl, aryl, heteroaryl, cycloalkyl or heterocycloalkyl group, in which C₁-C₆-alkyl, cycloalkyl and heterocycloalkyl are, independently of each other, optionally substituted one or more times with a halogen atom, an —OH or C₁-C₆-alkoxy group;
each occurrence of R^{c} is, independently, a hydrogen atom, a —C(═O)R^{e}, —S(═O)₂R^{e}, C₁-C₆-alkyl, C₁-C₆-haloalkyl cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl, are, independently of each other, optionally substituted one or more times with a halogen atom, an —OH, aryl, —OR^{f}, —NR^{d1}R^{d2}, or —OP(═O)(OR^{f})₂ group;
in each occurrence of R^{d1}, R^{d2}, R^{d1}, R^{d2} are, independently of each other, a hydrogen atom, a C₁-C₆-alkyl, cycloalkyl, heterocycloalkyl, aryl, heteroaryl, —C(═O)R^{e}, —S(═O)₂R^{e}, or —C(═O)NR^{g1}R^{g2} group, in which C₁-C₆-alkyl, cycloalkyl, heterocycloalkyl, aryl, or heteroaryl are, independently of each other, optionally substituted one or more times, the same way or differently, with a halogen atom, an —OH or aryl, —NR^{g1}R^{g2}, —OR^{f}, —C(═O)R^{e}, —S(═O)₂R^{e}, or —OP(═O)(OR^{f})₂ group;
or
R^{d1} and R^{d2}, together with the nitrogen atom to which they are bound, form a 3-, 4-, 5-, 6-, 7-, 8-, 9-, or 10-membered heterocycloalkyl ring, which is optionally substituted one or more times, the same way or differently, with a halogen atom, a C₁-C₆-alkyl, —NR^{g1}R^{g2}, —OR^{f}, —C(═O)R^{e}, —S(═O)₂R^{e}, or —OP(═O)(OR^{f})₂ group; and the carbon backbone of which is optionally interrupted one or more times, in the same way or differently, with NH, NR^{d3}, O, or S, and is optionally interrupted one or more times, in the same way or differently, with a —C(═O)—, —S(═O)—, and/or —S(═O)₂— group, and optionally contains one or more double bonds;
R^{d3} is a hydrogen atom, a C₁-C₆-alkyl, cycloalkyl, heterocycloalkyl, aryl, or heteroaryl group, in which C₁-C₆-alkyl or cycloalkyl are, independently of each other, optionally substituted one or more times with a halogen atom, an —OH, C₁-C₆-alkyl, cycloalkyl, C₁-C₆-haloalkyl or C₁-C₆-alkoxy group;
R^{e} is an —NR^{g1}R^{g2}, C₁-C₆-alkyl, cycloalkyl, C₁-C₆-alkoxy, aryl or heteroaryl group;
R^{f} is a hydrogen atom, a —C(═O)R^{e}, C₁-C₆-alkyl, C₁-C₆-haloalkyl, cycloalkyl, heterocycloalkyl, aryl, or heteroaryl group, in which C₁-C₆-alkyl, C₁-C₆-haloalkyl, cycloalkyl, heterocycloalkyl, aryl, or heteroaryl are, independently of each other, optionally substituted one or more times with a halogen atom, an —OH, C₁-C₆-alkoxy, aryl, or —NR^{g1}R^{g2} group;
R^{g1}, R^{g2}, are, independently of each other, a hydrogen atom, a C₁-C₆-alkyl, cycloalkyl, heterocycloalkyl, aryl, or heteroaryl group; or
R^{g1} and R^{g2}, together with the nitrogen atom to which they are bound, form a 3-, 4-, 5-, 6-, 7-, 8-, 9- or 10-membered heterocycloalkyl ring, which is optionally substituted one or more times, in the same way or differently, with a halogen atom, an —OH, C₁-C₆-alkyl, C₁-C₆-alkoxy group; and the carbon backbone of which is optionally interrupted one or more times, in the same way or differently, with NH, NR^{a}, O, S, and is optionally interrupted one or more times, in the same way or differently, with a —C(═O)—, —S(═O)—, and/or —S(═O)₂— group, and optionally contains one or more double bonds;
with the proviso that:
X-R⁶ is not (O or NH)—(CH₂)ᵣ—R^{r},
where R^{r} is NR^{s1}R^{s2} in which
r=1-4, and
R^{s1}, R^{s2}=independently hydrogen, C₁-C₈ alkyl, or taken together with the nitrogen to which they are attached, form a 3-10 member cyclic ring optionally containing one oxygen atom or one sulfur atom or one NH or N—C₁-C₈ alkyl group;
or a tautomer, stereoisomer, physiologically acceptable salt, hydrate, solvate, metabolite, or prodrug thereof.

5. The compound according to claim 1, which is selected from the group consisting of 5-fluoro-3-[(2-fluoro-4-iodophenyl)amino]-2-nitrophenoxybutane-1,2-diol;
5-fluoro-N-(2-fluoro-4-iodophenyl)-2-nitro-3-(2-piperidin-4-ylethoxy)aniline;
2-(3,4-dihydroxybutoxy)-4-fluoro-6-[(2-fluoro-4-iodophenyl)amino]benzonitrile
2-[2-(2,2-dimethyl-1,3-dioxolan-4-yl)ethoxy]-4-fluoro-6-[(2-fluoro-4-iodophenyl)amino]benzamide;
2-(3,4-dihydroxybutoxy)-4-fluoro-6-[(2-fluoro-4-iodophenyl)amino]benzamide;
5-fluoro-3-[(2-fluoro-4-iodophenyl)amino]-2-nitrophenoxypropane-1,2-diol;
5-fluoro-3-[(2-fluoro-4-iodophenyl)amino]-2-nitrophenoxypentane-1,2-diol;
2-(2,3-dihydroxypropoxy)-4-fluoro-6-[(2-fluoro-4-iodophenyl)amino]benzonitrile;
2-[(4,5-dihydroxypentyl)oxy]-4-fluoro-6-[(2-fluoro-4-iodophenyl)amino]benzonitrile;
2-[(4S)-2,2-dimethyl-1,3-dioxolan-4-yl]propoxy-4-fluoro-6-[(2-fluoro-4-iodophenyl)amino]benzamide;
2-[(3R)-3,4-dihydroxybutyl]oxy-4-fluoro-6-[(2-fluoro-4-iodophenyl)amino]benzamide;
2-[(3S)-3,4-dihydroxybutyl]oxy-4-fluoro-6-[(2-fluoro-4-iodophenyl)amino]benzamide;
2-[(4S)-4,5-dihydroxypentyl]oxy-4-fluoro-6-[(2-fluoro-4-iodophenyl)amino]benzamide;
2-{[(3R)-3,4-dihydroxybutyl]oxy}-4-fluoro-6-[(4-iodophenyl)amino]benzamide;
2-[(2-chloro-4-iodophenyl)amino]-6-{[(3R)-3,4-dihydroxybutyl]oxy}-4-fluorobenzamide;
2-{[(3R)-3,4-dihydroxybutyl]oxy}-4-fluoro-6-[(4-iodo-2-methylphenyl)amino]benzamide;
2-[(2-cyano-4-iodophenyl)amino]-6-{[(3R)-3,4-dihydroxybutyl]oxy}-4-fluorobenzamide;
2-[(4-bromo-2-fluorophenyl)amino]-6-{[(3R)-3,4-dihydroxybutyl]oxy}-4-fluorobenzamide;
2-[(4-bromo-2-chlorophenyl)amino]-6-{[(3R)-3,4-dihydroxybutyl]oxy}-4-fluorobenzamide;
2-{[(3R)-3,4-dihydroxybutyl]oxy}-6-[(4-ethynyl-2-fluorophenyl)amino]-4-fluorobenzamide;
2-{[(3R)-3,4-dihydroxybutyl]oxy}-4-fluoro-6-[(2-fluoro-4-iodophenyl)amino]-N-methylbenzamide;
2-{[(3R)-3,4-dihydroxybutyl]oxy}-N-ethyl-4-fluoro-6-[(2-fluoro-4-iodophenyl)amino]benzamide;
2-{[(3R)-3,4-dihydroxybutyl]amino}-4-fluoro-6-[(2-fluoro-4-iodophenyl)amino]benzamide;
2-{[(3R)-3,4-dihydroxybutyl](methyl)amino}-4-fluoro-6-[(2-fluoro-4-iodophenyl)amino]benzamide;
4-fluoro-2-[(2-fluoro-4-iodophenyl)amino]-6-{[(2S,3S)-2,3,4-trihydroxybutyl]oxy}benzamide; or
4-fluoro-2-[(2-fluoro-4-iodophenyl)amino]-6-{[(2R,3R)-2,3,4-trihydroxybutyl]oxy}benzamide;
or a physiologically acceptable salt, solvate, hydrate or stereoisomer thereof.

6. The compound according to claim 1, which is selected from the group consisting of:
N′-[3-[2-cyano-5-fluoro-3-[(2-fluoro-4-iodophenyl)amino]phenoxy]phenyl]-N,N-dimethyl-sulfamide;
{3-[2-Cyano-5-fluoro-3-(2-fluoro-4-iodo-phenylamino)-phenoxy]-phenyl}-carbamic acid tert-butyl ester;
2-(Cyclopent-3-enyloxy)-4-fluoro-6-(2-fluoro-4-iodo-phenylamino)-benzonitrile;
4-Fluoro-2-(2-fluoro-4-iodo-phenylamino)-6-[3-(4-methyl-piperazin-1-yl)-propoxy]-benzonitrile;
4-Fluoro-2-(2-fluoro-4-iodo-phenylamino)-6-(4-methyl-pent-3-enyloxy)-benzonitrile;
4-Fluoro-2-(2-fluoro-4-iodo-phenylamino)-6-(3-methyl-but-3-enyloxy)-benzonitrile;
4-Fluoro-2-(2-fluoro-4-iodo-phenylamino)-6-(2-imidazol-1-yl-ethoxy)-benzonitrile;
2-[3-(1,1-Dioxo-1λ⁶-thiomorpholin-4-yl)-propoxy]-4-fluoro-6-(2-fluoro-4-iodo-phenylamino)-benzonitrile;
4-Fluoro-2-(2-fluoro-4-iodo-phenylamino)-6-(2-pyridin-3-yl-ethoxy)-benzonitrile;
4-Fluoro-2-(2-fluoro-4-iodo-phenylamino)-6-[3-(2-oxo-pyrrolidin-1-yl)-propoxy]-benzonitrile;
3-[2-Cyano-5-fluoro-3-(2-fluoro-4-iodo-phenylamino)-phenoxymethyl]-pyrrolidine-1-carboxylic acid tert-butyl ester;
2-{2-[2-Cyano-5-fluoro-3-(2-fluoro-4-iodo-phenylamino)-phenoxy]-ethyl}-piperidine-1-carboxylic acid tert-butyl ester;
3-[2-Cyano-5-fluoro-3-(2-fluoro-4-iodo-phenylamino)-phenoxymethyl]-piperidine-1-carboxylic acid tert-butyl ester;
2-[2-Cyano-5-fluoro-3-(2-fluoro-4-iodo-phenylamino)-phenoxymethyl]-morpholine-4-carboxylic acid tert-butyl ester;
3-[2-Cyano-5-fluoro-3-(2-fluoro-4-iodo-phenylamino)-phenoxymethyl]-azetidine-1-carboxylic acid tert-butyl ester;
4-[2-Cyano-5-fluoro-3-(2-fluoro-4-iodo-phenylamino)-phenoxy]-piperidine-1-carboxylic acid tert-butyl ester;
{3-[2-Carbamoyl-5-fluoro-3-(2-fluoro-4-iodo-phenylamino)-phenoxy]-phenyl}-carbamic acid tert-butyl ester;
2-[3-[[(di methylamino)sulfonyl]amino]phenoxy]-4-fluoro-6-[(2-fluoro-4-iodophenyl)amino]-benzamide;
4-Fluoro-2-(2-fluoro-4-iodo-phenylamino)-6-[3-(2-oxo-pyrrolidin-1-yl)-propoxy]-benzamide;
2-[3-(1,1-Dioxo-1λ⁶-thiomorpholin-4-yl)-propoxy]-4-fluoro-6-(2-fluoro-4-iodo-phenylamino)-benzamide;
4-Fluoro-2-(2-fluoro-4-iodo-phenylamino)-6-(2-pyridin-3-yl-ethoxy)-benzamide;
4-Fluoro-2-(2-fluoro-4-iodo-phenylamino)-6-(4-methyl-pent-3-enyloxy)-benzamide;
4-Fluoro-2-(2-fluoro-4-iodo-phenylamino)-6-(3-methyl-but-3-enyloxy)-benzamide;
4-Fluoro-2-(2-fluoro-4-iodo-phenylamino)-6-((2S,3S)-2,3,4-trihydroxy-butoxy)-benzamide;
2-(Cyclopent-3-enyloxy)-4-fluoro-6-(2-fluoro-4-iodo-phenylamino)-benzamide;
3-[2-Carbamoyl-5-fluoro-3-(2-fluoro-4-iodo-phenylamino)-phenoxymethyl]-pyrrolidine-1-carboxylic acid tert-butyl ester;
2-{2-[2-Carbamoyl-5-fluoro-3-(2-fluoro-4-iodo-phenylamino)-phenoxy]-ethyl}-piperidine-1-carboxylic acid tert-butyl ester;
3-[2-Carbamoyl-5-fluoro-3-(2-fluoro-4-iodo-phenylamino)-phenoxymethyl]-piperidine-1-carboxylic acid tert-butyl ester;
2-[2-Carbamoyl-5-fluoro-3-(2-fluoro-4-iodo-phenylamino)-phenoxymethyl]-morpholine-4-carboxylic acid tert-butyl ester;
3-[2-Carbamoyl-5-fluoro-3-(2-fluoro-4-iodo-phenylamino)-phenoxymethyl]-azetidine-1-carboxylic acid tert-butyl ester;
{3-[2-Carbamoyl-5-fluoro-3-(2-fluoro-4-iodo-phenylamino)-phenoxy]-propyl}-carbamic acid tert-butyl ester;
4-[2-Carbamoyl-5-fluoro-3-(2-fluoro-4-iodo-phenylamino)-phenoxy]-piperidine-1-carboxylic acid tert-butyl ester;
4-Fluoro-2-(2-fluoro-4-iodo-phenylamino)-6-((2R,3R)-2,3,4-trihydroxy-butoxy)-benzamide;
2-(3-Amino-phenoxy)-4-fluoro-6-(2-fluoro-4-iodo-phenylamino)-benzamide;
4-Fluoro-2-(2-fluoro-4-iodo-phenylamino)-6-(pyrrolidin-3-ylmethoxy)-benzamide;
4-Fluoro-2-(2-fluoro-4-iodo-phenylamino)-6-(piperidin-3-ylmethoxy)-benzamide;
4-Fluoro-2-(2-fluoro-4-iodo-phenylamino)-6-(morpholin-2-ylmethoxy)-benzamide;
4-Fluoro-2-(2-fluoro-4-iodo-phenylamino)-6-(2-piperidin-2-yl-ethoxy)-benzamide;
2-(Azetidin-3-ylmethoxy)-4-fluoro-6-(2-fluoro-4-iodo-phenylamino)-benzamide;
4-Fluoro-2-(2-fluoro-4-iodo-phenylamino)-6-(piperidin-4-yloxy)-benzamide;
4-Fluoro-2-(2-fluoro-4-iodo-phenylamino)-6-(1H-indol-6-yloxy)-benzamide;
2-[3-(3,3-Dimethyl-ureido)-phenoxy]-4-fluoro-6-(2-fluoro-4-iodo-phenylamino)-benzamide;
2-(3,3-Dioxo-2,3-dihydro-3λ⁶-benzo[1,3]oxathiol-5-yloxy)-4-fluoro-6-(2-fluoro-4-iodo-phenylamino)-benzamide;
4-Fluoro-2-(2-fluoro-4-iodo-phenylamino)-6-phenoxy-benzamide;
4-Fluoro-2-(2-fluoro-4-iodo-phenylamino)-6-((1S,2S)-2-hydroxy-cyclopentyloxy)-benzamide;
4-Fluoro-2-(2-fluoro-4-iodo-phenylamino)-6-(4-imidazol-1-yl-phenoxy)-benzamide;
4-Fluoro-2-(2-fluoro-4-iodo-phenylamino)-6-(3-nitro-phenoxy)-benzamide;
2-(Benzo[1,3]dioxol-5-yloxy)-4-fluoro-6-(2-fluoro-4-iodo-phenylamino)-benzamide;
Dimethyl-carbamic acid 3-[2-carbamoyl-5-fluoro-3-(2-fluoro-4-iodo-phenylamino)-phenoxy]-phenyl ester;
2-(4-Acetylamino-phenoxy)-4-fluoro-6-(2-fluoro-4-iodo-phenylamino)-benzamide;
4-Fluoro-2-(2-fluoro-4-iodo-phenylamino)-6-(1-methyl-piperidin-4-yloxy)-benzamide;
4-{2-[2-Carbamoyl-5-fluoro-3-(2-fluoro-4-iodo-phenylamino)-phenoxy]-ethyl}-piperazine-1-carboxylic acid tert-butyl ester;
6-[2-Carbamoyl-5-fluoro-3-(2-fluoro-4-iodo-phenylamino)-phenoxy]-indole-1-carboxylic acid tert-butyl ester;
4-Fluoro-2-(2-fluoro-4-iodo-phenylamino)-6-[4-(methanesulfonyl-methyl-amino)-phenoxy]-benzamide;
4-Fluoro-2-(2-fluoro-4-iodo-phenylamino)-6-(pyridin-4-yloxy)-benzamide;
4-Fluoro-2-(2-fluoro-4-iodo-phenylamino)-6-(3-hydrazinocarbonyl-phenoxy)-benzamide;
Acetic acid 4-[2-carbamoyl-5-fluoro-3-(2-fluoro-4-iodo-phenylamino)-phenoxy]-cyclopent-2-enyl ester;
4-Fluoro-2-(2-fluoro-4-iodo-phenylamino)-6-(4-hydroxy-cyclopent-2-enyloxy)-benzamide;
Dimethyl-sulfamic acid 3-[2-carbamoyl-5-fluoro-3-(2-fluoro-4-iodo-phenylamino)-phenoxy]-phenyl ester;
2-[2-((S)-2,2-Dimethyl-[1,3]dioxolan-4-yl)-ethoxy]-4-fluoro-6-(2-fluoro-4-iodo-phenylamino)-benzamide;
4-Fluoro-2-(2-fluoro-4-iodo-phenylamino)-6-(3-methanesulfonylamino-phenoxy)-benzamide;
4-Fluoro-2-(2-fluoro-4-iodo-phenylamino)-6-(3-bis-methanesulfonyl-amino-phenoxy)-benzamide;
2-{2-[2-Carbamoyl-5-fluoro-3-(2-fluoro-4-iodo-phenylamino)-phenoxy]-ethyl}-piperidine-1-carboxylic acid dimethylamide;
2-[3-[[(propylamino)sulfonyl]amino]phenoxy]-4-fluoro-6-[(2-fluoro-4-iodophenyl)amino]-benzamide;
2-(3-Acetylamino-phenoxy)-4-fluoro-6-(2-fluoro-4-iodo-phenylamino)-benzamide;
2-[3-(3-Chloro-propane-1-sulfonylamino)-phenoxy]-4-fluoro-6-(2-fluoro-4-iodo-phenylamino)-benzamide;
2-[3-(1,1-Dioxo-1λ⁶-isothiazolidin-2-yl)-phenoxy]-4-fluoro-6-(2-fluoro-4-iodo-phenylamino)-benzamide;
2-[3-[[(amino)sulfonyl]amino]phenoxy]-4-fluoro-6-[(2-fluoro-4-iodophenyl)amino]-benzamide;
4-Fluoro-2-(2-fluoro-4-iodo-phenylamino)-6-(3-formylamino-phenoxy)-benzamide;
2-[2-(1-Ethanesulfonyl-piperidin-2-yl)-ethoxy]-4-fluoro-6-(2-fluoro-4-iodo-phenylamino)-benzamide;
2-[2-(1-Dimethylsulfamoyl-piperidin-2-yl)-ethoxy]-4-fluoro-6-(2-fluoro-4-iodo-phenylamino)-benzamide;
2-(3-Benzenesulfonylamino-propoxy)-4-fluoro-6-(2-fluoro-4-iodo-phenylamino)-benzamide;
2-(3-Benzoylamino-propoxy)-4-fluoro-6-(2-fluoro-4-iodo-phenylamino)-benzamide;
4-Fluoro-2-(2-fluoro-4-iodo-phenylamino)-6-[3-(3-phenyl-ureido)-propoxy]-benzamide;
2-(1-Benzenesulfonyl-piperidin-3-ylmethoxy)-4-fluoro-6-(2-fluoro-4-iodo-phenylamino)-benzamide;
4-Fluoro-2-(2-fluoro-4-iodo-phenylamino)-6-(1-methanesulfonyl-piperidin-3-ylmethoxy)-benzamide;
4-Fluoro-2-(2-fluoro-4-iodo-phenylamino)-6-[3-(pyridin-3-ylmethanesulfonylamino)-propoxy]-benzamide;
4-Fluoro-2-(2-fluoro-4-iodo-phenylamino)-6-[3-(1-methyl-1H-imidazole-4-sulfonylamino)-propoxy]-benzamide;
4-Fluoro-2-(2-fluoro-4-iodo-phenylamino)-6-[3-(1-methyl-1H-pyrazole-4-sulfonylamino)-propoxy]-benzamide;
4-Fluoro-2-(2-fluoro-4-iodo-phenylamino)-6-(3-trifluoromethanesulfonylamino-propoxy)-benzamide;
2-(1-Ethanesulfonyl-piperidin-3-ylmethoxy)-4-fluoro-6-(2-fluoro-4-iodo-phenylamino)-benzamide;
2-(1-Dimethylsulfamoyl-piperidin-3-ylmethoxy)-4-fluoro-6-(2-fluoro-4-iodo-phenylamino)-benzamide;
4-Fluoro-2-(2-fluoro-4-iodo-phenylamino)-6-[2-(1-methanesulfonyl-piperidin-2-yl)-ethoxy]-benzamide;
4-Fluoro-2-(2-fluoro-4-iodo-phenylamino)-6-(1-methanesulfonyl-pyrrolidin-3-ylmethoxy)-benzamide;
4-Fluoro-2-(2-fluoro-4-iodo-phenylamino)-6-(1-methanesulfonyl-piperidin-4-yloxy)-benzamide;
4-[2-Carbamoyl-5-fluoro-3-(2-fluoro-4-iodo-phenylamino)-phenoxy]-piperidine-1-carboxylic acid dimethylamide;
4-Fluoro-2-(2-fluoro-4-iodo-phenylamino)-6-[3-(morpholine-4-sulfonylamino)-phenoxy]-benzamide;
4-Fluoro-2-(2-fluoro-4-iodo-phenylamino)-6-[1-(1H-imidazole-4-sulfonyl)-azetidin-3-ylmethoxy]-benzamide;
4-Fluoro-2-(2-fluoro-4-iodo-phenylamino)-6-(1-methanesulfonyl-azetidin-3-ylmethoxy)-benzamide;
2-(1-Dimethylsulfamoyl-azetidin-3-ylmethoxy)-4-fluoro-6-(2-fluoro-4-iodo-phenylamino)-benzamide;
2-(3,4-Dihydroxy-4-methyl-pentyloxy)-4-fluoro-6-(2-fluoro-4-iodo-phenylamino)-benzamide;
2-(3,4-Dihydroxy-3-methyl-butoxy)-4-fluoro-6-(2-fluoro-4-iodo-phenylamino)-benzamide;
2-(3,4-Dihydroxy-4-methyl-pentyloxy)-4-fluoro-6-(2-fluoro-4-iodo-phenylamino)-benzamide (Enantiomer 1);
2-(3,4-Dihydroxy-4-methyl-pentyloxy)-4-fluoro-6-(2-fluoro-4-iodo-phenylamino)-benzamide (Enantiomer 2);
2-(3,4-Dihydroxy-3-methyl-butoxy)-4-fluoro-6-(2-fluoro-4-iodo-phenylamino)-benzamide (Enantiomer 1);
2-(3,4-Dihydroxy-3-methyl-butoxy)-4-fluoro-6-(2-fluoro-4-iodo-phenylamino)-benzamide (Enantiomer 2);
2-((1S,3S,4R)-3,4-Dihydroxy-cyclopentyloxy)-4-fluoro-6-(2-fluoro-4-iodo-phenylamino)-benzamide;
2-((1S,3S,4R)-3,4-Dihydroxy-cyclopentyloxy)-4-fluoro-6-(2-fluoro-4-iodo-phenylamino)-benzamide;
2-(3,4-Dihydroxy-4-methyl-pentyloxy)-4-fluoro-6-(2-fluoro-4-iodo-phenylamino)-benzonitrile;
2-(3,4-Dihydroxy-3-methyl-butoxy)-4-fluoro-6-(2-fluoro-4-iodo-phenylamino)-benzonitrile;
2-((S)-3,4-Dihydroxy-butoxy)-4-fluoro-6-(2-fluoro-4-iodo-phenylamino)-benzamide;
2-((R)-3,4-Dihydroxy-butoxy)-4-fluoro-6-(2-fluoro-phenylamino)-benzamide;
2-(4-Chloro-2-fluoro-phenylamino)-6-((R)-3,4-dihydroxy-butoxy)-4-fluoro-benzamide;
2-(4-Bromo-2-fluoro-phenylamino)-6-((R)-3,4-dihydroxy-butoxy)-4-fluoro-benzamide;
2-((R)-3,4-Dihydroxy-butoxy)-4-fluoro-6-(4-iodo-phenylamino)-benzamide;
2-((R)-3,4-Dihydroxy-butoxy)-4-fluoro-6-(2-fluoro-4-iodo-phenylamino)-benzamide;
2-((R)-3,4-Dihydroxy-butoxy)-6-(4-ethynyl-2-fluoro-phenylamino)-4-fluoro-benzamide;
2-((R)-3,4-Dihydroxy-butoxy)-4-fluoro-6-[2-fluoro-4-(4-hydroxy-but-1-ynyl)-phenylamino]-benzamide;
2-((R)-3,4-Dihydroxy-4-methyl-pentyloxy)-6-(4-ethynyl-2-fluoro-phenylamino)-4-fluoro-benzamide;
2-[3-[[(dimethylamino)sulfonyl]amino]phenoxy]-4-fluoro-6-[(4-ethynyl-2-fluorophenyl)amino]-benzamide;
2-[3-[[(propylamino)sulfonyl]amino]phenoxy]-4-fluoro-6-[(4-ethynyl-2-fluorophenyl)amino]-benzamide;
Methanesulfonic acid (R)-4-[2-carbamoyl-5-fluoro-3-(2-fluoro-4-iodo-phenylamino)-phenoxy]-2-hydroxy-butyl ester;
4-Fluoro-2-(2-fluoro-4-iodo-phenylamino)-6-[(R)-3-hydroxy-4-(2-hydroxy-ethylamino)-butoxy]-benzamide;
2-((R)-4-Amino-3-hydroxy-butoxy)-4-fluoro-6-(2-fluoro-4-iodo-phenylamino)-benzamide;
4-Fluoro-2-(2-fluoro-4-iodo-phenylamino)-6-{(R)-3-hydroxy-4-[(2-methoxy-ethyl)-methyl-amino]-butoxy}-benzamide;
2-((R)-4-Diethylamino-3-hydroxy-butoxy)-4-fluoro-6-(2-fluoro-4-iodo-phenylamino)-benzamide;
4-Fluoro-2-(2-fluoro-4-iodo-phenylamino)-6-((R)-3-hydroxy-4-morpholin-4-yl-butoxy)-benzamide;
2-((R)-4-Ethylamino-3-hydroxy-butoxy)-4-fluoro-6-(2-fluoro-4-iodo-phenylamino)-benzamide;
4-Fluoro-2-(2-fluoro-4-iodo-phenylamino)-6-((R)-3-hydroxy-4-piperidin-1-yl-butoxy)-benzamide;
4-Fluoro-2-(2-fluoro-4-iodo-phenylamino)-6-[(R)-3-hydroxy-4-(2-methoxy-ethylamino)-butoxy]-benzamide;
2-((R)-3,4-Dihydroxy-butoxy)-6-(2-fluoro-4-iodo-phenylamino)-benzamide;
4-Bromo-2-((R)-3,4-dihydroxy-butoxy)-6-(2-fluoro-4-iodo-phenylamino)-benzamide;
4-Chloro-2-((R)-3,4-dihydroxy-butoxy)-6-(2-fluoro-4-iodo-phenylamino)-benzamide;
2-((R)-3,4-Dihydroxy-butoxy)-6-(2-fluoro-4-iodo-phenylamino)-4-methoxy-benzamide;
3-Chloro-6-((R)-3,4-dihydroxy-butoxy)-2-(2-fluoro-4-iodo-phenylamino)-benzamide;
2-((R)-3,4-Dihydroxy-butoxy)-4-fluoro-6-(2-fluoro-4-iodo-phenylamino)-benzoic acid;
4-Fluoro-2-(2-fluoro-4-iodo-phenylamino)-6-[3-(2,2,2-trifluoro-acetylamino)-phenoxy]-benzamide;
2-((R)-3,4-Dihydroxy-butoxy)-4-fluoro-6-(3-fluoro-biphenyl-4-ylamino)-benzamide;
2-((R)-4-Chloro-3-hydroxy-butoxy)-4-fluoro-6-(2-fluoro-4-iodo-phenylamino)-benzamide;
4-Fluoro-2-(2-fluoro-4-iodo-phenylamino)-6-((R)-3-hydroxy-4-imidazol-1-yl-butoxy)-benzamide; compound with 2,4,6-triisopropyl-benzenesulfonic acid;
2-((R)-3,4-Dimethoxy-butoxy)-4-fluoro-6-[(2-fluoro-4-iodo-phenyl)-methyl-amino]-N,N-dimethyl-benzamide;
2-((R)-3,4-Dihydroxy-butoxy)-4-fluoro-6-[(2-fluoro-4-iodo-phenyl)-methyl-amino]-N,N-dimethyl-benzamide;
2-((R)-3,4-Dihydroxy-butoxy)-4-fluoro-6-[(2-fluoro-4-iodo-phenyl)-methyl-amino]-N-methyl-benzamide;
2-((R)-3,4-Dihydroxy-butoxy)-4-fluoro-6-(2-fluoro-4-iodo-phenylamino)-N-methyl-benzamide;
N-Benzyl-2-((R)-3,4-dihydroxy-butoxy)-4-fluoro-6-(2-fluoro-4-iodo-phenylamino)-benzamide;
2-((R)-3,4-Dihydroxy-butoxy)-4-fluoro-6-(2-fluoro-4-iodo-phenylamino)-benzonitrile;
Phthalic acid mono-{(R)-4-[2-cyano-5-fluoro-3-(2-fluoro-4-iodo-phenylamino)-phenoxy]-2-hydroxy-butyl}ester;
4-Fluoro-2-(2-fluoro-4-iodo-phenylamino)-6-(3-oxo-butoxy)-benzonitrile;
4-Fluoro-2-(2-fluoro-4-iodo-phenylamino)-6-((2R,3R)-2,3,4-trihydroxy-butoxy)-benzonitrile;
2-(3,4-Dihydroxy-phenoxy)-4-fluoro-6-(2-fluoro-4-iodo-phenylamino)-benzamide; and
2-[3-(3,3-Dimethyl-ureido)-phenoxy]-4-fluoro-6-(2-fluoro-4-iodo-phenylamino)-benzamide
or a physiologically acceptable salt, solvate, hydrate or stereoisomer thereof.

7. A method of preparing a compound of general formula (I) according to claim 1, said method comprising the step of allowing an intermediate compound of general formula Ia: 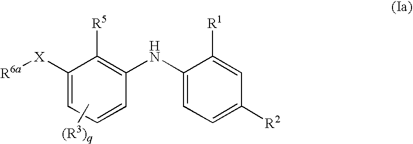
in which R¹, R², R³, R⁵, R^{6a}, X and q are as defined in claim 1, to react with an acid, for example hydrochloric acid or TFA thereby giving a compound of formula I 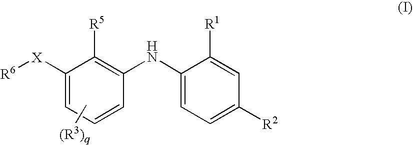
in which R¹, R², R³, R⁵, R⁶, X and q are as defined in claim 1.

8. A method of preparing a compound of general formula (Ic) according to claim 1, said method comprising the step of allowing an intermediate compound of general formula 1b: 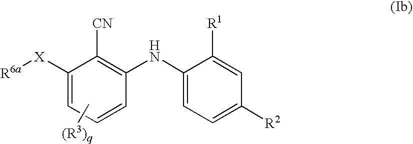
in which R¹, R², R³, R^{6a}, X and q are as defined in claim 1, to react with an acid, for example hydrochloric acid or TFA thereby giving a compound of formula Ic: 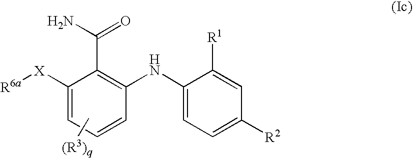
in which R¹, R², R³, R^{6a}, X and q are as defined in claim 1.

9. A method of preparing a compound of general formula (Ig) according to claim 1, said method comprising the step of allowing an intermediate compound of general formula 1f: 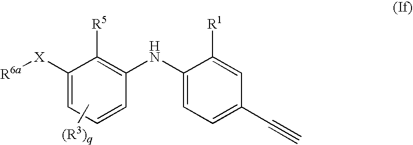
in which R¹, R³, R⁵, R⁶, X and q are as defined in claim 1, to react with a deprotecting agent thereby giving a compound of formula Ig: 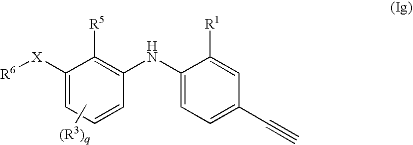
in which R¹, R³, R⁵, R⁶, X and q are as defined in claim 1.

10. A method of preparing a compound of general formula (It) according to claim 1, said method comprising the step of allowing an intermediate compound of general formula Is: 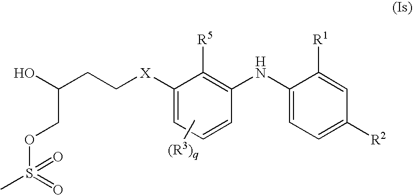
in which R¹, R², R³, R⁵ and q are as defined in claim 1, to react either in situ or after isolation with an amine of general formula (IX) to afford a compound of Formula (It): 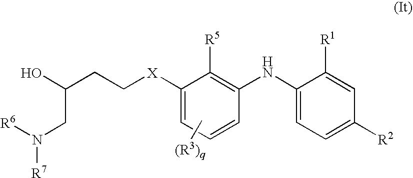
in which R¹, R³, R³, R⁵, R⁶, R⁷, X and q are as defined in claim 1.

11. A pharmaceutical composition comprising a compound according to claim 1, or a tautomer, stereoisomer, physiologically acceptable salt, hydrate, solvate, metabolite, or prodrug thereof, and a pharmaceutically acceptable diluent or carrier.

12. The pharmaceutical composition according to claim 11 wherein said compound is present in a therapeutically effective amount.

13. The pharmaceutical composition according to claim 12 which further comprises at least one further active compound.

14. The pharmaceutical composition according to claim 13, in which said further active compound is an anti-hyperproliferative agent, an anti-angiogenic agent, a mitotic inhibitor, an alkylating agent, an anti-metabolite, a DNA-intercalating antibiotic, a growth factor inhibitor, a cell cycle inhibitor, an enzyme inhibitor, a toposisomerase inhibitor, a biological response modifier, or an anti-hormone.

15. A packaged pharmaceutical composition comprising a container, the pharmaceutical composition of claim 11, and instructions for using the pharmaceutical composition to treat a disease or condition in a mammal.

16. A method of inhibiting mitogen extracellular kinase enzymes in a cell comprising contacting a cell with one or more compounds according to claim 1.

17. The method according to claim 16, wherein said cell is a mammalian cell.

18. Use of a compound according to claim 1 for the preparation of a medicament for treating a hyperproliferative disorder or abnormal cell growth in a mammal.

19. The use according to claim 18, wherein said hyperproliferative disorder is cancer.

20. The use according to claim 19, wherein said cancer is a cancer of the breast, respiratory tract, brain, reproductive organs, digestive tract, urinary tract, eye, liver, skin, head and neck, endocrine system or a distant metastasis of a solid tumor.

21. The use according to claim 20, wherein said cancer is a sarcoma, a melanoma or a hematological malignancy.

22. The use according to claim 21, wherein said haematological malignancy is lymphoma, leukaemia or multiple myeloma.

23. Use of a compound according to claim 1 for the preparation of a medicament for treating an angiogenesis disorder in a mammal.

24. The use according to claim 23, wherein said hyperproliferative disorder is psoriasis, restenosis, autoimmune disease, atherosclerosis, rheumatoid arthritis, chronic pain, neuropathic pain, osteoarthritis, benign prostate hyperplasia, hyperproliferative disease of the eye.

25. The use according to claim 24, wherein said hyperproliferative disease of the eye is cataract, conjunctival epithelial cell hypermitosis or goblet cell hyperplasia.
